SEQUENCE LISTING (10 sequences)

SEQ ID NO: 1 - protein, 8 aa, Artificial Sequence
  Ublituximab Variable heavy chain CDR1
GYTFTSYN

SEQ ID NO: 2 - protein, 8 aa, Artificial Sequence
  Ublituximab Variable heavy chain CDR2
IYPGNGDT

SEQ ID NO: 3 - protein, 11 aa, Artificial Sequence
  Ublituximab Variable heavy chain CDR3
ARYDYNYAMDY

SEQ ID NO: 4 - protein, 118 aa, Artificial Sequence
  Ublituximab Variable heavy chain
QAYLQQSGAELVRPGASVKMSCKASGYTFTSYNMHWVKQTPRQGLEWIGGIYPGNGDTSYNQKFKGKATLTVGKSSSTAYMQLSSLTSEDSAVYFCARYDYNYAMDYWGQGTSVTVSS

SEQ ID NO: 5 - protein, 330 aa, Artificial Sequence [sequence omitted]

SEQ ID NO: 6 - protein, 5 aa, Artificial Sequence
  Ublituximab Variable light chain CDR1
SSVSY

SEQ ID NO: 7 - protein, 3 aa, Artificial Sequence
  Ublituximab Variable light chain CDR2
ATS

SEQ ID NO: 8 - protein, 9 aa, Artificial Sequence
  Ublituximab Variable light chain CDR3
QQWTFNPPT

SEQ ID NO: 9 - protein, 106 aa, Artificial Sequence
  Ublituximab Variable light chain
QIVLSQSPAILSASPGEKVTMTCRASSSVSYMHWYQQKPGSSPKPWIYATSNLASGVPARFSGSGSGTSYSFTISRVEAEDAATYYCQQWTFNPPTFGGGTRLEIK

SEQ ID NO: 10 - protein, 106 aa, Artificial Sequence
  Ublituximab Constant light chain
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2022/0143026 A1
(43) Pub. Date: May 12, 2022

(54) TRIPLE COMBINATION TO TREAT B-CELL MALIGNANCIES

(71) Applicants: TG Therapeutics, Inc., New York, NY (US); Rhizen Pharmaceuticals SA, La Chaux de Fonds (CH); LABORATOIRE FRANCAIS DU FRACTIONNEMENT ET DES BIOTECHNOLOGIES, Les Ulis (FR)

(72) Inventors: Michael S. WEISS, New York, NY (US); Hari P. MISKIN, New York, NY (US); Peter SPORTELLI, New York, NY (US)

(21) Appl. No.: 17/525,731

(22) Filed: Nov. 12, 2021

Related U.S. Application Data
(60) Provisional application No. 63/243,708, filed on Sep. 13, 2021.
(60) Provisional application No. 63/113,189, filed on Nov. 12, 2020.

(51) Int. Cl. A61K 31/519; A61K 39/395; A61K 31/5025; C07K 16/28; A61K 45/06; A61P 35/00

(52) U.S. Cl. CPC A61K 31/519; A61K 39/3955; A61P 35/00; C07K 16/2887; A61K 45/06; A61K 31/5025

(57) ABSTRACT

Methods for treating B-cell malignancies are provided comprising administering a triple combination of agents, comprising: (i) a PI3K-delta selective inhibitor (e.g., umbralisib); (ii) an anti-CD20 antibody (e.g., ublituximab); and (iii) the Bruton's tyrosine kinase (BTK) inhibitor TG-1701. Kits for carrying out the claimed methods are also provided.

### CROSS-REFERENCE TO RELATED APPLICATIONS

This application claims the benefit under 35 U.S.C. § 119(e) of U.S. Provisional Application No. 63/243,708, filed Sep. 13, 2021 and U.S. Provisional Application No. 63/113,189, filed Nov. 12, 2020, the contents of which are hereby incorporated by reference in their entireties.

### REFERENCE TO SEQUENCE LISTING SUBMITTED ELECTRONICALLY

The content of the electronically submitted sequence listing in ASCII text file (Name: 3261_0150002_Seqlisting_ST25.txt; Size: 7,777 bytes; and Date of Creation: Nov. 12, 2021), filed with the application, is incorporated herein by reference in its entirety.

### BACKGROUND OF THE INVENTION

### Field of the Invention

The present invention relates generally to the field of cancer therapy. More particularly, the present invention relates to methods and kits for treating B-cell malignancies, by administering to a subject a combination of agents comprising: (i) an inhibitor of PI3 kinase (PI3K)-delta; (ii) an anti-CD20 antibody; and (iii) an inhibitor of Bruton's tyrosine kinase (BTK), wherein the BTK inhibitor is TG-1701.

### Background Art

Despite more than a century of dedicated scientific and clinical research, curing cancer remains one of the biggest medical challenges to date. Cancer treatments have mainly relied on the combination of surgery, radiotherapy, and/or cytotoxic chemotherapies. Within the last decade, however, targeted cancer therapies have opened a new era in the field of oncology. Targeted cancer therapies are drugs designed to interfere with specific molecules necessary for tumor growth and progression; they are broadly classified into monoclonal antibodies (mAbs) or small molecules. Some examples of targeted therapies include monoclonal antibodies to CD20 (e.g., rituximab/Rituxan® for treating lymphomas), CD52 (e.g., alemtuzumab/Campath®), VEGF (e.g., bevacizumab/Avastin®), HER2 (e.g., trastuzumab/Herceptin® for treating Her2+ breast and stomach cancers), EGFR (e.g., cetuximab/Erbitux® for treating colorectal cancer), CTLA-4 (e.g., ipilimumab/Yervoy® for treating melanoma), and PD-1 (e.g., MDX-1106, CT-011). Small molecule therapies target dysregulated pathways of cancer cells, e.g., RAS, RAF, PI3K, MEK, JAK, STAT, and BTK. For example, BTK inhibition, such as with ibrutinib (Imbruvica®) has been effective in the treatment of chronic lymphocytic leukemia (CLL) and B-cell lymphoma, but requires continuous treatment and complete responses (CR) are rare. Further, significant toxicities with prolonged use of ibrutinib (median of six months) have been observed, and may limit ibrutinib use. See, Byrd, J. et al, “Long-term efficacy and safety with ibrutinib (ibr) in previously treated chronic lymphocytic leukemia (CLL): up to four years follow-up of the RESONATE study,” J Clin Oncol. 35(suppl), Abstract 7510 (2017).

BTK-based combination therapies have the potential to increase the depth of response and to permit time-limited therapy. A chemo-free triplet combination of TGR-1202, ublituximab, and ibrutinib was well-tolerated and highly active in patients with advanced CLL and NHL. See, Nastoupil, L. et al., 22^{nd} Congress of the European Hematology Association (EHA); Abstract 5772 (2017). See also, WO2017/205843 and U.S. Patent Appl. Publ. No. US 2019/0175592. Notably, several combination studies of ibrutinib plus rituximab failed to show an improvement in progression-free survival over ibrutinib alone. See, Woyach J. A. et al., N Engl J Med. 379:2517-2528 (2018); Burger, J. A. et al., Blood 130:427 (2017).

While effective B-cell cancer therapies exist (e.g., Rituxan®, Imbruvica®), suboptimal or non-durable responses, the need for continuous treatment, and/or resistance to one or more therapeutic agents have remained a challenge. Accordingly, there is a need in the art for more effective, safe, shorter, and durable combination therapies for the treatment of B-cell malignancies.

### BRIEF SUMMARY OF THE INVENTION

The triplet combination treatment described herein is suitable for treating B-cell malignancies.

In one aspect, provided herein are methods of treating a B-cell malignancy in a subject in need thereof, comprising, (a) administering to the subject a combination of agents, in therapeutically effective amounts, said combination of agents comprising:

(i) a PI3K-delta selective inhibitor of Formula A, or a stereoisomer thereof, or a pharmaceutically acceptable salt, solvate, or prodrug thereof:

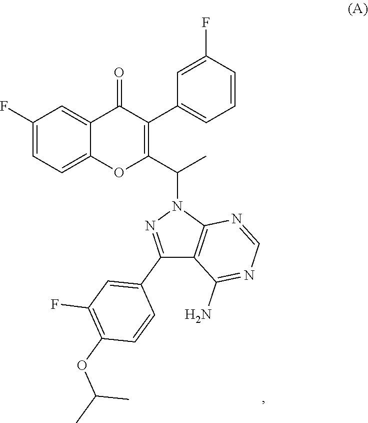 [SMILES: CC(C)Oc1ccc(-c2nn(C(C)c3oc4ccc(F)cc4c(=O)c3-c3cccc(F)c3)c3ncnc(N)c23)cc1F]

selected from one or more of:
(RS)-2-(1-(4-amino-3-(3-fluoro-4-isopropoxyphenyl)-1H-pyrazolo[3,4-d]pyrimidin-1-yl)ethyl)-6-fluoro-3-(3-fluorophenyl)-4H-chromen-4-one;
(S)-2-(1-(4-amino-3-(3-fluoro-4-isopropoxyphenyl)-1H-pyrazolo[3,4-d]pyrimidin-1-yl)ethyl)-6-fluoro-3-(3-fluorophenyl)-4H-chromen-4-one; and
(R)-2-(1-(4-amino-3-(3-fluoro-4-isopropoxyphenyl)-1H-pyrazolo[3,4-d]pyrimidin-1-yl)ethyl)-6-fluoro-3-(3-fluorophenyl)-4H-chromen-4-one;

(ii) an anti-CD20 antibody, wherein the anti-CD20 antibody is ublituximab or an anti-CD20 antibody or antibody fragment that binds to the same epitope as ublituximab; and

(iii) a BTK inhibitor, wherein the BTK inhibitor is (R)-4-amino-1-(1-(but-2-ynoyl)pyrrolidin-3-yl)-3-(4-(2,6-difluorophenoxy)phenyl)-1,6-dihydro-7H-pyrrolo[2,3-d]pyridazin-7-one (TG-1701), or an isomer, polymorph, enantiomer, pharmaceutically acceptable salt, solvate, or prodrug thereof; and

(b) treating said subject with a B-cell malignancy.

In some aspects, the PI3K-delta selective inhibitor is administered at a dosage from: about 200 mg to about 1200 mg, about 400 mg to about 1000 mg, about 400 mg to about 800 mg, about 400 mg, about 500 mg, about 600 mg, about 700 mg, about 800 mg, about 900 mg, about 1000 mg, or about 1200 mg.

In some aspects, the PI3K-delta selective inhibitor is administered daily.

In some aspects, the PI3K-delta selective inhibitor is formulated for oral administration.

In certain aspects, the PI3K-delta selective inhibitor is (S)-2-(1-(4-amino-3-(3-fluoro-4-isopropoxyphenyl)-1H-pyrazolo[3,4-d]pyrimidin-1-yl)ethyl)-6-fluoro-3-(3-fluorophenyl)-4H-chromen-4-one (umbralisib).

In certain aspects, the PI3K-delta selective inhibitor is (S)-2-(1-(4-amino-3-(3-fluoro-4-isopropoxyphenyl)-1H-pyrazolo[3,4-d]pyrimidin-1-yl)ethyl)-6-fluoro-3-(3-fluorophenyl)-4H-chromen-4-one p-toluenesulfonic acid salt (umbralisib PTSA salt).

In some aspects, the umbralisib is administered orally at a dose from about 400 mg to about 1200 mg per day.

In certain aspects, the umbralisib is administered orally at a dose of about 400 mg per day. In certain aspects, the umbralisib is administered orally at a dose of 600 mg per day. In certain aspects, the umbralisib is administered orally at a dose of 800 mg per day.

In some aspects, the anti-CD20 antibody is ublituximab. In some aspects, the ublituximab comprises the VH CDR1, CDR2, and CDR3 region of sequences SEQ ID NOS: 1, 2, and 3, and the VL CDR1, CDR2, and CDR3 region of sequences SEQ ID NOS: 6, 7, and 8. In some aspects, ublituximab comprises the VH of SEQ ID NO: 4 and the VL of SEQ ID NO: 9.

In some aspects, the ublituximab is administered at a dose from: about 450 mg to about 1200 mg, about 600 to about 1200 mg, about 600 to about 1000 mg, about 600 to about 900 mg, about 600 mg, about 700 mg, about 800 mg, or about 900 mg about once every 1 to 9 weeks, about once every week, about twice every week, about once every 2 weeks, about once every 3 weeks, about once every 4 weeks, about once every 5 weeks, about once every 6 weeks, about once every 7 weeks, about once every 8 week, or about once every 9 weeks.

In certain aspects, the ublituximab is administered at a dose of about 900 mg.

In some aspects, the ublituximab is administered intravenously.

In some aspects, the ublituximab is administered on days 1, 8, and 15 of cycle 1 and day 1 of cycles 2, 3, 4, 5, 6, and every 3 months thereafter, wherein each cycle is about 28 days.

In some aspects, the BTK inhibitor TG-1701 or an isomer, polymorph, enantiomer, pharmaceutically acceptable salt, solvate, or prodrug thereof is administered orally once daily at a dosage from: about 100 mg, about 200 mg, about 300 mg, or about 400 mg. In certain aspects, the TG-1701 or an isomer, polymorph, enantiomer, pharmaceutically acceptable salt, solvate, or prodrug thereof is administered orally once daily at a dosage of about 300 mg or about 400 mg per day. In certain aspects, the TG-1701 or an isomer, polymorph, enantiomer, pharmaceutically acceptable salt, solvate, or prodrug thereof is administered orally once daily at a dosage of about 300 mg per day. In certain aspects, the TG-1701 or an isomer, polymorph, enantiomer, pharmaceutically acceptable salt, solvate, or prodrug thereof is administered orally once daily at a dosage of about 400 mg per day.

In certain aspects, the TG-1701 or an isomer, polymorph, enantiomer, pharmaceutically acceptable salt, solvate, or prodrug thereof is in crystalline form.

In some aspects, the subject with a B-cell malignancy is a human subject. In certain aspects, the human subject has a B-cell malignancy selected from the group consisting of acute lymphocytic leukemia (ALL), acute myeloid leukemia (AML), chronic lymphocytic leukemia (CLL), small lymphocytic lymphoma (SLL), multiple myeloma (MM), non-Hodgkin's lymphoma (NHL), mantle cell lymphoma (MCL), follicular lymphoma (FL), Waldenstrom's macroglobulinemia (WM), diffuse large B-cell lymphoma (DLBCL), marginal zone lymphoma (MZL), Burkitt's lymphoma, hairy cell leukemia (HCL), and Richter's transformation (RT).

In certain aspects, the B-cell malignancy is selected from the group consisting of chronic lymphocytic leukemia (CLL), follicular lymphoma (FL), mantle cell lymphoma (MCL), small lymphocytic lymphoma (SLL), diffuse large B-cell lymphoma (DLBCL), Waldenstrom's macroglobulinemia (WM), and marginal zone lymphoma (MZL).

In certain aspects, the B-cell malignancy is CLL.

In some aspects, the B-cell malignancy overexpresses CD20.

In some aspects, the B-cell malignancy is refractory to chemotherapy.

In some aspects, the B-cell malignancy is refractory to an anti-CD20 antibody, a PI3K-delta inhibitor, or a BTK inhibitor, administered as a monotherapy.

In certain aspects, the B-cell malignancy is refractory to a non-umbralisib PI3K-delta inhibitor. In certain aspects, the B-cell malignancy is refractory to a non-ublituximab anti-CD20 antibody. In certain embodiments, the non-ublituximab anti-CD20 antibody is rituximab. In certain aspects, the B-cell malignancy is refractory to a non-TG-1701 BTK inhibitor. In certain aspects, the non-TG-1701 BTK inhibitor is ibrutinib.

In some aspects, the B-cell malignancy has relapsed.

In some aspects, the human subject has one or more genetic mutations selected from the group consisting of 17p del, 11q del, p53, unmutated IgVH together with ZAP-70+ and/or CD38+, trisomy 12, and complex karyotype.

In some aspects, the combination of agents i, ii, and iii are administered separately. In certain aspects, the combination of agents i, ii, and iii are administered sequentially.

In some aspects, a complete anti-tumor response is observed following administration of all agents i, ii, and iii to said subject. In some aspects, a partial anti-tumor response is observed following administration of all agents i, ii, and iii to said subject. In some aspects, a very good partial anti-tumor response is observed following administration of all agents i, ii, and iii to said subject.

In some aspects, the duration of the anti-tumor response is about 24 weeks to about 36 months.

In some aspects, the anti-tumor response is determined by percent reduction in tumor burden from baseline. In certain aspects, the percent reduction in tumor burden from baseline is about 25%-100%. In certain aspects, the percent reduction in tumor burden from baseline is about 50%.

In some aspects, agents i and iii are orally administered simultaneously or sequentially once a day. In certain aspects, agents i and iii are contained in the same pharmaceutical composition.

In some aspects, the method of treating a B-cell malignancy further comprises administering at least one additional therapeutic agent.

In certain aspects, the at least one additional therapeutic agent is selected from the group consisting of mitotic inhibitors, alkylating agents, anti-metabolites, anthracyclines, vinca alkaloids, plant alkaloids, nitrogen mustards, proteasome inhibitors, intercalating antibiotics, growth factor inhibitors, cell-cycle inhibitors, biological response modifiers, anti-hormones, angiogenesis inhibitors, anti-androgens, DNA interactive agents, purine analogues, topoisomerase I inhibitors, topoisomerase II inhibitors, tubulin interacting agents, hormonal agents, thymidylate synthase inhibitors, non-BTK and non-PI3K-delta tyrosine kinase inhibitors, angiogenesis inhibitors, EGF inhibitors, VEGF inhibitors, CDK inhibitors, SRC inhibitors, c-Kit inhibitors, Her1/2 inhibitors, BET bromodomain inhibitors, inhibitors of myc, anti-tumor antibodies, monoclonal antibodies directed against growth factor receptors, monoclonal antibodies directed against checkpoint inhibitors, monoclonal antibodies against CD19 and/or CD47, protein kinase modulators, radioactive isotopes, immunotherapies, glucocorticoids, and any combinations thereof.

In certain aspects, the at least one additional therapeutic agent is selected from the group consisting of a proteasome inhibitor, Bortezomib (Velcade®), Carfilzomib (PR-171), PR-047, disulfiram, lactacystin, PS-519, eponemycin, epoxomycin, aclacinomycin, CEP-1612, MG-132, CVT-63417, PS-341, vinyl sulfone tripeptide inhibitors, ritonavir, PI-083, (+/−)-7-methylomuralide, (−)-7-methylomuralide, lenalidomide, bendamustine, TG-1501, TG-1601, TG-1801, and any combinations thereof.

In certain aspects, the at least one additional therapeutic agent is combination chemotherapy selected from the group consisting of cyclophosphamide, doxorubicin, vincristine, and prednisone (CHOP); rituxan, cyclophosphamide, doxorubicin, vincristine, and prednisone (R-CHOP); ifosfamide, carboplatin, and etoposide (ICE); rituxan, ifosfamide, carboplatin, and etoposide (R-ICE); rituximab, doxorubicin, cyclophosphamide, vindesine, bleomycin and prednisone (R-ACVBP); dose-adjusted etoposide, doxorubicin, cyclophosphamide, vincristine, prednisone and rituximab (DA-EPOCH-R); dexamethasone, cytarabine, and cisplatin (DHAP); bendamustine and rituximab (R-bendamustine); and gemcitabine and oxaliplatin, with or without rituximab (GemOx or R-GemOx).

In another aspect, provided herein are kits comprising: (a) a combination of agents (i)-(iii), as described herein; and (b) instructions for using said agents in combination.

In some aspects, the kit comprises ublituximab, umbralisib, and TG-1701.

In some aspects, the kit further comprises one or more additional therapeutic agents that can be used to treat B-cell malignancies.

### BRIEF DESCRIPTION OF THE DRAWINGS/FIGURES

FIG. 1 is a flow chart of the dose-escalation phase of a Phase 1/1b clinical trial for TG-1701 monotherapy and TG-1701+Umbralisib+Ublituximab (TG-1701+U2) combination therapy, and for disease-specific cohorts: CLL, WM, and MCL that is described in Example 1.

FIG. 2 is a flow chart depicting trial design in the dose-escalation phase of a Phase 1/1b clinical trial for TG-1701 monotherapy and TG-1701+Umbralisib+Ublituximab (TG-1701+U2) combination therapy, and for disease-specific cohorts: CLL, WM, and MCL that is described in Example 1.

FIG. 3 is a bar graph showing the best percent change in tumor burden from baseline (efficacy) in 22 patients in the dose-escalation TG-1701 monotherapy arm (100-400 mg) of the Phase 1/1b clinical trial described in Example 1. The patients included those with the following B-cell malignancies: DLBCL=2 patients; WM/LPL=10 patients; FL=2 patients; MZL=1 patient; CLL=5 patients; SLL=1 patient; and MCL=1 patient. One patient with CLL and two patients with MZL do not have target lesions.

FIG. 4 is a bar graph showing the best percent change in tumor burden from baseline (efficacy) in patients in the disease-specific cohorts (CLL, MCL, and WM/LPL) in the TG-1701 monotherapy arm (200 mg) of the Phase 1/1b clinical trial described in Example 1. The patients included those with the following B-cell malignancies: CLL=20 patients; MCL=18 patients; and WM/LPL=19 patients. One patient with MCL did not have target lesions. Treatment naïve patients in each cohort are designated by an asterisk (*).

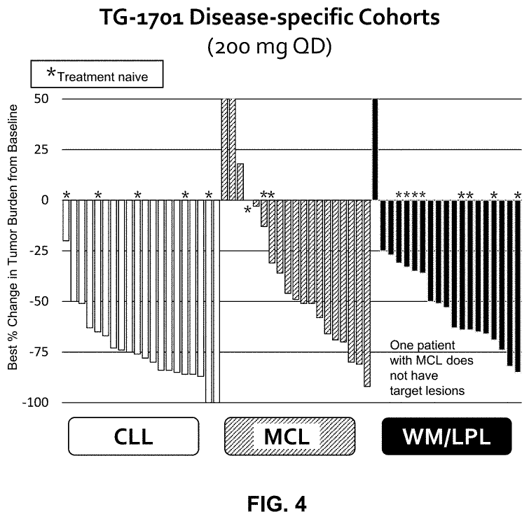

FIG. 5 is a bar graph showing the best percent change in tumor burden from baseline (efficacy) in 14 patients in the dose-escalation TG-1701+U2 combination arm of the Phase 1/1b clinical trial described in Example 1. The patients included those with the following B-cell malignancies: DLBCL=1 patient; WM/LPL=1 patient; FL=7 patients; MZL=2 patients; CLL=3 patients. FIG. 5 shows that 3 of the 14 patients in the combination therapy arm with TG-1701+U2 had a complete response (CR).

FIG. 6 is a bar graph showing treatment exposure and response duration in 41 patients in the dose-escalation phase of the TG-1701 monotherapy and the TG-1701+U2 combination arms of the Phase 1/1b clinical trial described in Example 1. The patients included: DLBCL=3 patients; WM=10 patients; FL=9 patients; SLL=1 patient; LPL=1 patient; MZL=7 patients; MCL=1 patient; CLL=9 patients. FIG. 6 shows that 3 patients had a complete response (CR) following combination therapy with TG-1701+U2. FIG. 6 also shows that 20 patients had a partial response (PR).

FIG. 7 is a waterfall graph showing efficacy in the dose escalation phase for monotherapy and combination therapy for the CLL subjects studied in Example 2. FIG. 7 shows the best percent change from baseline in tumor burden for 8 of 9 evaluable CLL subjects in the dose escalation (DE) cohort—5 patients received TG-1701 monotherapy and 3 patients received combination therapy. One patient that received TG-1701 monotherapy did not have target lesions; however, was considered a responder by >50% reduction in lymphocytosis. One patient that received TG-1701+U2 triplet recently enrolled and was too early to evaluate; therefore, they were not considered evaluable for efficacy.

FIG. 8 shows the best percent change from baseline in tumor burden for 20 subjects in the CLL disease-specific cohort receiving 200 mg TG-1701 (* indicates the 5 patients that were treatment naïve (TN)) and for 19 of 20 subjects in the CLL disease-specific cohort receiving 300 mg TG-1701. One patient did not have disease assessment (death due to COVID prior to first assessment). (* indicates the 4 patients that were TN).

FIG. 9 is a swimmer plot showing treatment exposure and status (on-going verses discontinued) for 50 CLL patients described in Example 2. The y-axis displays the respective dose levels of TG-1701 received (*indicates patients that were TN). The x-axis indicates duration of therapy for each patient. The Figure legend describes and distinguishes the respective cohorts.

FIG. 10 is a bar graph showing the best percent change in tumor burden from baseline (efficacy) in 19 patients in the dose-escalation TG-1701+U2 combination arm of the Phase 1/1b clinical trial described in Example 3. The patients included those with the following B-cell malignancies: DLBCL=1 patient; WM/LPL=1 patient; FL=8 patients; MZL=4 patients; CLL=3 patients. FIG. 10 shows that 4 of the 19 patients in the combination therapy arm with TG-1701+U2 had a complete response (CR), as indicated by asterisks (*).

### DETAILED DESCRIPTION OF THE INVENTION

### Definitions

To facilitate an understanding of the present invention, a number of terms and phrases are defined below.

The term “CD20” (also known as B lymphocyte CD20 antigen, MS4A1, B lymphocyte surface antigen B1, Bp35, and Leukocyte surface antigen Leu-16) refers to any native CD20, unless otherwise indicated. As used herein, the term “CD20” encompasses “full-length,” unprocessed CD20, as well as any form of CD20 that results from processing within the cell. The term also encompasses naturally occurring variants of CD20, e.g., splice variants, allelic variants, and isoforms. The CD20 polypeptides described herein can be isolated from a variety of sources, such as from human tissue types or from another source, or prepared by recombinant or synthetic methods. Examples of CD20 sequences include, but are not limited to, NCBI reference numbers NP_068769.2 and NP_690605.1.

The term “antibody” means an immunoglobulin molecule that recognizes and specifically binds to a target, such as a protein, polypeptide, peptide, carbohydrate, polynucleotide, lipid, or combinations of the foregoing through at least one antigen recognition site within the variable region of the immunoglobulin molecule. As used herein, the term “antibody” encompasses intact polyclonal antibodies, intact monoclonal antibodies, antibody fragments (such as Fab, Fab′, F(ab′)2, and Fv fragments), single chain Fv (scFv) mutants, multispecific antibodies such as bispecific antibodies generated from at least two intact antibodies, chimeric antibodies, humanized antibodies, human antibodies, fusion proteins comprising an antigen determination portion of an antibody, and any other modified immunoglobulin molecule comprising an antigen recognition site so long as the antibodies exhibit the desired biological activity. An antibody can be of any of the five major classes of immunoglobulins: IgA, IgD, IgE, IgG, and IgM, or subclasses (isotypes) thereof (e.g., IgG1, IgG2, IgG3, IgG4, IgA1, and IgA2), based on the identity of their heavy-chain constant domains referred to as alpha, delta, epsilon, gamma, and mu, respectively. The different classes of immunoglobulins have different and well known subunit structures and three-dimensional configurations. Antibodies can be naked or conjugated to other molecules such as toxins, radioisotopes, etc.

A “blocking” antibody or an “antagonist” antibody is one which inhibits or reduces biological activity of the antigen it binds, such as CD20. In a certain embodiment, blocking antibodies or antagonist antibodies substantially or completely inhibit the biological activity of the antigen. Desirably, the biological activity is reduced by 10%, 20%, 30%, 50%, 70%, 80%, 90%, 95%, or even 100%.

The term “anti-CD20 antibody” or “an antibody that binds to CD20” refers to an antibody that is capable of binding CD20 with sufficient affinity such that the antibody is useful as a diagnostic and/or therapeutic agent in targeting CD20. The extent of binding of an anti-CD20 antibody to an unrelated, non-CD20 protein is less than about 10% of the binding of the antibody to CD20 as measured, e.g., by a radioimmunoassay (MA). In certain aspects, an antibody that binds to CD20 has a dissociation constant (Kd) of ≤1 μM, ≤100 nM, ≤10 nM, ≤1 nM, or ≤0.1 nM.

The term “antibody fragment” refers to a portion of an intact antibody and refers to the antigenic determining variable regions of an intact antibody. Examples of antibody fragments include, but are not limited to, Fab, Fab′, F(ab′)2, and Fv fragments, linear antibodies, single chain antibodies, and multispecific antibodies formed from antibody fragments.

A “monoclonal antibody” refers to a homogeneous antibody population involved in the highly specific recognition and binding of a single antigenic determinant, or epitope. This is in contrast to polyclonal antibodies that typically include different antibodies directed against different antigenic determinants. The term “monoclonal antibody” encompasses both intact and full-length monoclonal antibodies, as well as antibody fragments (such as Fab, Fab′, F(ab′)2, Fv), single chain (scFv) mutants, fusion proteins comprising an antibody portion, and any other modified immunoglobulin molecule comprising an antigen recognition site. Furthermore, “monoclonal antibody” refers to such antibodies made in any number of manners including but not limited to by hybridoma, phage selection, recombinant expression, and transgenic animals.

The term “humanized antibody” refers to forms of non-human (e.g., murine) antibodies that are specific immunoglobulin chains, chimeric immunoglobulins, or fragments thereof that contain minimal non-human (e.g., murine) sequences. Typically, humanized antibodies are human immunoglobulins in which residues from the complementary determining region (CDR) are replaced by residues from the CDR of a non-human species (e.g., mouse, rat, rabbit, hamster) that have the desired specificity, affinity, and capability (Jones et al., Nature 321:522-525 (1986); Riechmann et al., Nature 332:323-327 (1988); Verhoeyen et al., Science 239:1534-1536 (1988)). In some instances, the Fv framework region (FR) residues of a human immunoglobulin are replaced with the corresponding residues in an antibody from a non-human species that has the desired specificity, affinity, and capability. The humanized antibody can be further modified by the substitution of additional residues either in the Fv framework region and/or within the replaced non-human residues to refine and optimize antibody specificity, affinity, and/or capability. In general, the humanized antibody will comprise substantially all of at least one, and typically two or three, variable domains containing all or substantially all of the CDR regions that correspond to the non-human immunoglobulin whereas all or substantially all of the FR regions are those of a human immunoglobulin consensus sequence. The humanized antibody can also comprise at least a portion of an immunoglobulin constant region or domain (Fc), typically that of a human immunoglobulin. Examples of methods used to generate humanized antibodies are described in U.S. Pat. No. 5,225,539 or 5,639,641.

A “variable region” of an antibody refers to the variable region of the antibody light chain or the variable region of the antibody heavy chain, either alone or in combination. The variable regions of the heavy and light chain each consist of four framework regions (FR) connected by three complementarity determining regions (CDRs), also known as hypervariable regions. The CDRs in each chain are held together in close proximity by the FRs and, with the CDRs from the other chain, contribute to the formation of the antigen-binding site of antibodies. There are at least two techniques for determining CDRs: (1) an approach based on cross-species sequence variability (i.e., Kabat et al., Sequences of Proteins of Immunological Interest, (5th ed., 1991, National Institutes of Health, Bethesda, Md.)); and (2) an approach based on crystallographic studies of antigen-antibody complexes (Al-lazikani et al, J. Molec. Biol. 273:927-948 (1997)). In addition, combinations of these two approaches are sometimes used in the art to determine CDRs.

The Kabat numbering system is generally used when referring to a residue in the variable domain (approximately residues 1-107 of the light chain and residues 1-113 of the heavy chain) (e.g., Kabat et al., Sequences of Immunological Interest. 5^{th} Ed. Public Health Service, National Institutes of Health, Bethesda, Md. (1991)).

The amino acid position numbering as in Kabat, refers to the numbering system used for heavy chain variable domains or light chain variable domains of the compilation of antibodies in Kabat et al., Sequences of Proteins of Immunological Interest, 5^{th} Ed. Public Health Service, National Institutes of Health, Bethesda, Md. (1991). Using this numbering system, the actual linear amino acid sequence can contain fewer or additional amino acids corresponding to a shortening of, or insertion into, a FR or CDR of the variable domain. For example, a heavy chain variable domain can include a single amino acid insert (residue 52a according to Kabat) after residue 52 of H2 and inserted residues (e.g., residues 82a, 82b, and 82c, etc. according to Kabat) after heavy chain FR residue 82. The Kabat numbering of residues can be determined for a given antibody by alignment at regions of homology of the sequence of the antibody with a “standard” Kabat numbered sequence. Chothia refers instead to the location of the structural loops (Chothia and Lesk, J. Mol. Biol. 196:901-917 (1987)). The end of the Chothia CDR-H1 loop, when numbered using the Kabat numbering convention, varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software.

| Loop | Kabat | AbM | Chothia |
|---|---|---|---|
| L1 | L24-L34 | L24-L34 | L24-L34 |
| L2 | L50-L56 | L50-L56 | L50-L56 |
| L3 | L89-L97 | L89-L97 | L89-L97 |
| H1 | H31-H35B | H26-H35B | H26-H32 . . . 34 |
| | | (Kabat Numbering) | |
| H1 | H31-H35 | H26-H35 | H26-H32 |
| | | (Clothia Numbering) | |
| H2 | H50-H65 | H50-H58 | H52-H56 |
| H3 | H95-H102 | H95-H102 | H95-H102 |

The term “human antibody” means an antibody produced by a human or an antibody having an amino acid sequence corresponding to an antibody produced by a human made using any technique known in the art. This definition of a human antibody includes intact or full-length antibodies, fragments thereof, and/or antibodies comprising at least one human heavy and/or light chain polypeptide such as, for example, an antibody comprising murine light chain and human heavy chain polypeptides.

The term “chimeric antibody” refers to an antibody wherein the amino acid sequence of the immunoglobulin molecule is derived from two or more species. Typically, the variable region of both light and heavy chains corresponds to the variable region of antibodies derived from one species of mammals (e.g., mouse, rat, rabbit, etc.) with the desired specificity, affinity, and capability, while the constant regions are homologous to the sequences in antibodies derived from another (usually human) to avoid eliciting an immune response in that species.

The terms “epitope” or “antigenic determinant” are used interchangeably herein and refer to that portion of an antigen capable of being recognized and specifically bound by a particular antibody. When the antigen is a polypeptide, epitopes can be formed both from contiguous amino acids and noncontiguous amino acids juxtaposed by tertiary folding of a protein. Epitopes formed from contiguous amino acids are typically retained upon protein denaturing, whereas epitopes formed by tertiary folding are typically lost upon protein denaturing. An epitope typically includes at least 3, and more usually, at least 5 or 8-10 amino acids in a unique spatial conformation.

“Binding affinity” generally refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present invention. Specific illustrative aspects are described herein.

The phrase “substantially similar,” or “substantially the same,” as used herein, denotes a sufficiently high degree of similarity between two numeric values (generally one associated with an antibody of the invention and the other associated with a reference/comparator antibody) such that one of skill in the art would consider the difference between the two values to be of little or no biological and/or statistical significance within the context of the biological characteristics measured by said values (e.g., Kd values). The difference between said two values is less than about 50%, less than about 40%, less than about 30%, less than about 20%, or less than about 10% as a function of the value for the reference/comparator antibody.

A polypeptide, antibody, polynucleotide, vector, cell, or composition which is “isolated” is a polypeptide, antibody, polynucleotide, vector, cell, or composition which is in a form not found in nature. Isolated polypeptides, antibodies, polynucleotides, vectors, cells or compositions include those which have been purified to a degree that they are no longer in a form in which they are found in nature.

The terms “cancer” and “cancerous” refer to or describe the physiological condition in mammals in which a population of cells are characterized by uncontrolled or unregulated cell growth. Examples of cancer include, e.g., carcinoma, lymphoma, blastoma, sarcoma, and leukemia.

The term “B-cell cancer” or “B-cell malignancy” refers to an uncontrolled or unregulated growth of B-cells in the blood, bone marrow, or lymph node. One skilled in the art would understand that a B-cell malignancy is a type of hematological malignancy that includes lymphomas, leukemias, and myelomas. The B-cell malignancy may be indolent or aggressive.

Non-limiting examples of B-cell malignancies that may be treated with the methods or kits of the invention include acute lymphocytic leukemia (ALL), acute myeloid leukemia (AML), chronic lymphocytic leukemia (CLL), small lymphocytic lymphoma (SLL), multiple myeloma (MM), non-Hodgkin's lymphoma (NHL), mantle cell lymphoma (MCL), follicular lymphoma (FL), Waldenstrom's macroglobulinemia (WM), diffuse large B-cell lymphoma (DLBCL), marginal zone lymphoma (MZL), which includes extranodal MZL, nodal MZL, and splenic MZL, hairy cell leukemia (HCL), Burkitt's lymphoma (BL), and Richter's transformation. In some aspects, the DLBCL is an activated B-cell DLBCL (ABC-DLBCL), a germinal center B-cell like DLBCL (GBC-DLBCL), a double hit DLBCL (DH-DLBCL), or a triple hit DLBCL (TH-DLBCL). In some aspects, certain CLLs (or other leukemias, such as the ones described herein) are considered “high risk” due to the presence of one of more genetic mutations. As used herein, “high risk” CLL, for example, means CLL characterized by at least one of the following genetic mutations: 17p del; 11q del; p53; unmutated IgVH together with ZAP-70+ and/or CD38+; and trisomy 12, and complex karyotype.

“Tumor” and “neoplasm” refer to any mass of tissue that result from excessive cell growth or proliferation, either benign (noncancerous) or malignant (cancerous) including pre-cancerous lesions.

The terms “cancer cell,” “tumor cell,” and grammatical equivalents refer to the total population of cells derived from a tumor or a pre-cancerous lesion, including both non-tumorigenic cells, which comprise the bulk of the tumor cell population, and tumorigenic stem cells (cancer stem cells). As used herein, the term “tumor cell” will be modified by the term “non-tumorigenic” when referring solely to those tumor cells lacking the capacity to renew and differentiate to distinguish those tumor cells from cancer stem cells.

The term “subject” refers to any animal (e.g., a mammal), including, but not limited to humans, non-human primates, rodents, and the like, which is to be the recipient of a particular treatment. Typically, the terms “subject” and “patient” are used interchangeably herein in reference to a human subject.

The term “pharmaceutical formulation” refers to a preparation which is in such form as to permit the biological activity of the active ingredient to be effective, and which contains no additional components which are unacceptably toxic to a subject to which the formulation would be administered. Such formulations can be sterile.

An “effective amount” of an antibody or an agent as disclosed herein, is an amount sufficient to carry out a specifically stated purpose. An “effective amount” can be determined empirically and in a routine manner by those skilled in the art, in relation to the stated purpose.

The term “therapeutically effective amount” refers to the amount of an agent (e.g., monoclonal antibody, small molecule, chemotherapeutic drug, etc. . . . ), as disclosed herein, that is effective to “treat” a disease or disorder in a subject or mammal. In the case of cancer, the therapeutically effective amount of the agent or drug can reduce the number of cancer cells; reduce the tumor size; inhibit (i.e., slow to some extent and in a certain aspect, stop) cancer cell infiltration into peripheral organs; inhibit (i.e., slow to some extent and in a certain aspect, stop) tumor metastasis; inhibit, to some extent, tumor growth; and/or relieve to some extent one or more of the symptoms associated with the cancer. See the definition herein of “treating.” To the extent the drug can prevent growth and/or kill existing cancer cells, it can be cytostatic and/or cytotoxic. A “prophylactically effective amount” refers to an amount effective, at dosages and for periods of time necessary, to achieve the desired prophylactic result. Typically, but not necessarily, since a prophylactic dose is used in subjects prior to or at an earlier stage of disease, the prophylactically effective amount will be less than the therapeutically effective amount.

Terms such as “treating,” “treatment,” “to treat,” “having a therapeutic effect,” alleviating,” “to alleviate,” or “slowing the progression of” refer to both 1) therapeutic measures that cure, slow down, lessen symptoms of, and/or halt progression of a diagnosed pathologic condition or disorder, such as a B-cell malignancy, and 2) prophylactic or preventative measures that prevent and/or slow the development of a targeted pathologic condition or disorder. Thus, those in need of treatment include those already with the disorder; those prone to have the disorder; and those in whom the disorder is to be prevented.

In certain aspects, a subject is successfully “treated” for a B-cell malignancy according to the methods of the present invention if the patient shows one or more of the following: reduction in cachexia, increase in survival time, elongation in time to tumor progression, reduction in tumor mass (e.g., by scan), reduction in tumor burden and/or a prolongation in time to tumor metastasis, time to tumor recurrence or progressive disease (PD), tumor response, complete response (CR), partial response (PR), stable disease (SD), progression free survival (PFS), overall survival (OS), each as measured by standards set by the National Cancer Institute and the U.S. Food and Drug Administration for the approval of new drugs. See, Johnson et al, J Clin. Oncol. 21:1404-1411 (2003). In some aspects, the “therapeutic effect,” as defined above, also encompasses a reduction in toxicity or adverse side effects, and/or an improvement in tolerability.

In certain aspects, guidelines provided by standard international workshops for particular B-cell malignancies are used to assess tumor response, such as, for CLL, as set forth in Hallek, M. et al., Blood 111:5446-5456 (2008); for NHL, as set forth in Cheson, B. D. et al., J Clin Oncol 25:579-586 (2007); and for WM, according to the sixth international workshop on WM, Owen, R. G. et al., Br J Haematol. 160:171-176 (2013).

In some aspects, treating the B-cell malignancy using the methods and kits described herein reduces percent tumor burden from baseline (i.e., prior to administration of the combination of agents described herein) by about 25%-100%. In some aspects, treating the B-cell malignancy using the methods and kits described herein reduces percent tumor burden from baseline by at least about 20%, by at least about 25%, by at least about 30%, by at least about 40%, by at least about 50%, by at least about 60%, by at least about 70%, by at least about 80%, by at least about 90%. In certain aspects, the methods and kits described herein promote B-cell malignancy regression to the point of eliminating the B-cell malignancy. In some aspects, subjects can be assessed for tumor burden or evaluated for anti-tumor response by e.g., CT, PET-CT, and/or MRI.

As used herein, “duration of the percent reduction in tumor burden” is the interval from the first documentation of complete response (CR) or partial response (PR) to the earlier of the first documentation of definitive disease progression or death from any cause. In certain aspects, the “duration of the reduction in percent tumor burden” can be observed and continue for a period of at least about 24 weeks to about 36 months.

A “combination” of an anti-CD20 antibody (e.g., ublituximab), a PI3K-delta selective inhibitor (e.g., umbralisib), and the BTK inhibitor TG-1701, is generally synonymous with a “combination of agents.” A “combination of agents” refers to the administration of at least one of each of these three agents (could be more than one type of each agent) to the same population of B-cells or to the same subject simultaneously, sequentially, or both simultaneously and sequentially. Thus, by way of example, administration of an anti-CD20 antibody preceding or following (e.g., by hour(s), day(s), week(s), or month(s)) administration of a PI3K-delta selective inhibitor, preceding or following (e.g., by hour(s), day(s), week(s), or month(s)) administration of BTK inhibitor TG-1701, constitutes administration of a combination of agents.

As will be apparent to one skilled in the art from the context, a “combination of agents” can also include an anti-CD20 antibody (e.g., ublituximab), a PI3K-delta selective inhibitor (e.g., umbralisib), and BTK inhibitor TG-1701, and one or more additional therapeutic agents, as described herein. In addition, simultaneous administration of an anti-CD20 antibody or fragment thereof, a PI3K-delta selective inhibitor, and a BTK inhibitor also constitutes administration of a combination of the anti-CD20 antibody or fragment thereof, PI3K-delta selective inhibitor, and BTK inhibitor TG-1701, regardless of whether the anti-CD20 antibody or fragment thereof, PI3K-delta inhibitor, and the BTK inhibitor are administered together in a single pharmaceutical formulation or are administered simultaneously in separate pharmaceutical formulations by either the same or different routes of administration. Further, the term “combination of agents” is intended to include treatment regimens in which the agents are administered by the same or different route of administration or at the same or different time.

As used herein, the term “U2” refers to the combination of Ublituximab+Umbralisib, as used in the methods or kits disclosed herein.

As used herein, the term “TG-1701+U2” refers to the triple combination of the BTK inhibitor TG-1701+Ublituximab+Umbralisib, as used in the methods or kits disclosed herein.

As used herein, an “adverse event” (AE) is any unfavorable and generally unintended or undesirable sign (including an abnormal laboratory finding), symptom, or disease associated with the use of a medical or pharmaceutical treatment.

As used herein, the term “induction” or “induction therapy” refers to the first agent, or combination of agents, as disclosed herein, administered to treat a B-cell proliferative disorder. If the first agent or combination of agents does not result in a complete response or it causes severe side effects, other agents may be added or used instead (see “consolidation”). Induction is also called primary therapy, or primary treatment, and is administered with the goal of inducing some initial reduction in disease burden. For example, induction therapy can be used in the methods described herein to include the use of an anti-CD20 antibody (e.g., ublituximab) and a PI3K delta inhibitor (e.g., umbralisib).

As used herein, “consolidation” or “consolidation therapy” refers to treatment that is given following induction therapy. Consolidation therapy is used to kill any malignant B− cells that may be left in the body following induction therapy. For example, if an anti-CD20 antibody (e.g., ublituximab) and a PI3K delta inhibitor (e.g., umbralisib) are used as induction therapy, consolidation therapy can include the use of BTK inhibitor TG-1701. Consolidation is also called intensification therapy.

As used herein, “maintenance” or “maintenance therapy” refers to treatment that is given to help keep the B-cell malignancy from returning after successful treatment with the initial therapy. Maintenance therapy may include treatment with the same agents that were used in the consolidation phase, and the agents in this phase may be administered for an extended period of time.

A B-cell malignancy which “does not respond,” “responds poorly,” or is “refractory” to treatment (with, for example, an anti-CD20 antibody) does not show statistically significant improvement in response to that treatment when compared to no treatment or treatment with a placebo in a recognized animal model or human clinical trial, or which responds to an initial treatment, but grows as treatment continues. In the clinical trial results discussed in Example 1, “refractory disease” was more particularly defined as disease that progressed within 6 months of their last prior therapy.

A B-cell malignancy which has “relapsed” means that the tumor has returned following treatment. In the clinical trial results discussed in Example 1, “relapsed disease” was more particularly defined as disease that progressed at least 6 months after prior therapy (so the patient was at least stable for 6 months after their last dose of therapy).

As used herein, the term “R/R” means that the B-cell malignancy is relapsed or refractory, or possibly both.

The terms “polypeptide,” “peptide,” and “protein” are used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that, because the polypeptides of this invention are based upon antibodies, in certain aspects, the polypeptides can occur as single chains or associated chains.

The terms “identical” or percent “identity” in the context of two or more polypeptides, refer to two or more sequences or subsequences that are the same or have a specified percentage of amino acid residues that are the same, when compared and aligned (introducing gaps, if necessary) for maximum correspondence, not considering any conservative amino acid substitutions as part of the sequence identity. The percent identity can be measured using sequence comparison software or algorithms or by visual inspection. Various algorithms and software are known in the art that can be used to obtain alignments of amino acid sequences.

One such non-limiting example of a sequence alignment algorithm is the algorithm described in Karlin et al, Proc. Natl. Acad. Sci., 87:2264-2268 (1990), as modified in Karlin et al., Proc. Natl. Acad. Sci., 90:5873-5877 (1993), and incorporated into the NBLAST and)(BLAST programs (Altschul et al., Nucleic Acids Res., 25:3389-3402 (1991)). In certain aspects, Gapped BLAST can be used as described in Altschul et al., Nucleic Acids Res., 25:3389-3402 (1997). BLAST-2, WU-BLAST-2 (Altschul et al., Methods in Enzymology, 266:460-480 (1996)), ALIGN, ALIGN-2 (Genentech, South San Francisco, Calif.) or Megalign (DNASTAR) are additional publicly available software programs that can be used to align sequences. In certain aspects, the percent identity between two nucleotide sequences is determined using the GAP program in GCG software (e.g., using a NWSgapdna.CMP matrix and a gap weight of 40, 50, 60, 70, or 90 and a length weight of 1, 2, 3, 4, 5, or 6). In certain alternative aspects, the GAP program in the GCG software package, which incorporates the algorithm of Needleman and Wunsch (J. Mol. Biol. 48:444-453 (1970)), can be used to determine the percent identity between two amino acid sequences (e.g., using either a Blossum 62 matrix or a PAM250 matrix, and a gap weight of 16, 14, 12, 10, 8, 6, or 4 and a length weight of 1, 2, 3, 4, 5). Alternatively, in certain aspects, the percent identity between amino acid sequences is determined using the algorithm of Myers and Miller (CABIOS, 4:11-17 (1989)). For example, the percent identity can be determined using the ALIGN program (version 2.0) and using a PAM120 with residue table, a gap length penalty of 12 and a gap penalty of 4. Appropriate parameters for maximal alignment by particular alignment software can be determined by one skilled in the art. In certain aspects, the default parameters of the alignment software are used. In certain aspects, the percentage identity “X” of a first amino acid sequence to a second sequence amino acid is calculated as 100×(Y/Z), where Y is the number of amino acid residues scored as identical matches in the alignment of the first and second sequences (as aligned by visual inspection or a particular sequence alignment program) and Z is the total number of residues in the second sequence.

In some aspects, two polypeptides of the invention are substantially identical, meaning they have at least 70%, at least 75%, at least 80%, at least 85%, at least 90%, and in some aspects at least 95%, 96%, 97%, 98%, 99% amino acid residue identity, when compared and aligned for maximum correspondence, as measured using a sequence comparison algorithm or by visual inspection. In certain aspects, identity exists over a region of the sequences that is at least about 10, about 20, about 40-60 residues in length or any integral value therebetween, or over a longer region than 60-80 residues, at least about 90-100 residues, or the sequences are substantially identical over the full length of the sequences being compared.

All numbers in this disclosure indicating amounts, ratios of materials, physical properties of materials, and/or use are to be understood as modified by the word “about,” except as otherwise explicitly indicated. When referring to a number or a numerical range, the term “about” means that the number or numerical range referred to is an approximation within experimental variability (or within statistical experimental error), and thus the number or numerical range can vary from, for example, between 1% and 15% of the stated number or numerical range.

The compounds of the invention can contain one or more asymmetric centers (chiral centers) and can thus give rise to enantiomers, diastereomers, and other stereoisomeric forms that can be defined, in terms of absolute stereochemistry, as (R)- or (S)-. The present disclosure is meant to encompass all such possible forms, as well as their racemic and resolved forms and mixtures thereof. The individual enantiomers can be separated according to methods known in the art in view of the present disclosure.

As used herein, the term “stereoisomers” is a general term for all isomers of individual molecules that differ only in the orientation of their atoms in space. It includes enantiomers and isomers of compounds with more than one chiral center that are not mirror images of one another (diastereomers).

The term “chiral center” refers to a carbon atom to which four different groups are attached.

The terms “enantiomer” and “enantiomeric” refer to a molecule that cannot be superimposed on its mirror image and hence is optically active wherein the enantiomer rotates the plane of polarized light in one direction and its mirror image compound rotates the plane of polarized light in the opposite direction.

The term “racemic” refers to a mixture of equal parts of enantiomers and which mixture is optically inactive.

The term “resolution” refers to the separation, concentration or depletion of one of the two enantiomeric forms of a molecule.

The present disclosure encompasses solvates of compounds of the invention. Solvates typically do not significantly alter the physiological activity or toxicity of the compounds, and as such may function as pharmacological equivalents. The term “solvate” as used herein is a combination, physical association and/or solvation of a compound of the present disclosure with a solvent molecule, e.g. a disolvate, monosolvate, or hemisolvate, where the ratio of solvent molecule to compound of the present disclosure is about 2:1, about 1:1 or about 1:2, respectively. This physical association involves varying degrees of ionic and covalent bonding, including hydrogen bonding. In certain instances, the solvate can be isolated, such as when one or more solvent molecules are incorporated into the crystal lattice of a crystalline solid. Thus, “solvate” encompasses both solution-phase and isolatable solvates. Compounds of the invention can be present as solvated forms with a pharmaceutically acceptable solvent, such as water, methanol, ethanol, and the like, and it is intended that the disclosure includes both solvated and unsolvated forms of compounds of the invention. One type of solvate is a hydrate. A “hydrate” relates to a particular subgroup of solvates where the solvent molecule is water. Solvates typically can function as pharmacological equivalents. Preparation of solvates is known in the art. See, e.g., Caira, M. et al., J. Pharmaceut. Sci. 93:601-611 (2004); van Tonder, E. C. et al., AAPS Pharm. Sci. Tech. 5(1): Article 12 (2004); and Bingham, A. L. et al., Chem. Commun. 603-604 (2001). A typical, non-limiting, process of preparing a solvate would involve dissolving a compound of the invention in a desired solvent (organic, water, or a mixture thereof) at temperatures about 20° C. to about 25° C., then cooling the solution at a rate sufficient to form crystals, and isolating the crystals by known methods, e.g., filtration. Analytical techniques such as infrared spectroscopy can be used to confirm the presence of the solvent in a crystal of the solvate.

The term “prodrug” refers to a compound, which is an inactive precursor of a compound, converted into its active form in the body by normal metabolic processes. Prodrug design is discussed generally in Hardman, J. G. et al. (eds.), Goodman and Gilman's The Pharmacological Basis of Therapeutics, 9^{th} ed., pp. 11-16 (1996). A thorough discussion is provided in Higuchi et al., Prodrugs as Novel Delivery Systems, Vol. 14, ASCD Symposium Series, and in Roche (ed.), Bioreversible Carriers in Drug Design, American Pharmaceutical Association and Pergamon Press (1987). To illustrate, prodrugs can be converted into a pharmacologically active form through hydrolysis of, for example, an ester or amide linkage, thereby introducing or exposing a functional group on the resultant product. The prodrugs can be designed to react with an endogenous compound to form a water-soluble conjugate that further enhances the pharmacological properties of the compound, for example, increased circulatory half-life. Alternatively, prodrugs can be designed to undergo covalent modification on a functional group with, for example, glucuronic acid, sulfate, glutathione, amino acids, or acetate. The resulting conjugate can be inactivated and excreted in the urine, or rendered more potent than the parent compound. High molecular weight conjugates also can be excreted into the bile, subjected to enzymatic cleavage, and released back into the circulation, thereby effectively increasing the biological half-life of the originally administered compound. Prodrugs of the compounds of the invention are intended to be covered within the scope of this invention.

The present invention also includes compounds which differ only in the presence of one or more isotopically enriched atoms, for example, replacement of hydrogen with deuterium or tritium, or the replacement of a carbon by ¹³C- or ¹⁴C-enriched carbon. The compounds of the present invention may also contain unnatural proportions of atomic isotopes at one or more of atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are encompassed within the scope of the present invention.

The present disclosure further encompasses salts of the compounds of the invention, including non-toxic pharmaceutically acceptable salts. Examples of pharmaceutically acceptable addition salts include inorganic and organic acid addition salts and basic salts. The pharmaceutically acceptable salts include, but are not limited to, metal salts such as sodium salt, potassium salt, cesium salt and the like; alkaline earth metals such as calcium salt, magnesium salt and the like; organic amine salts such as triethylamine salt, pyridine salt, picoline salt, ethanolamine salt, triethanolamine salt, dicyclohexylamine salt, N,N′-dibenzylethylenediamine salt and the like; inorganic acid salts such as hydrochloride, hydrobromide, phosphate, sulphate and the like; organic acid salts such as citrate, lactate, tartrate, maleate, fumarate, mandelate, acetate, dichloroacetate, trifluoroacetate, oxalate, formate, succinates, palmoates, benzoates, salicylates, ascorbates, glycerophosphates, ketoglutarates and the like; sulfonates such as methanesulfonate, benzenesulfonate, p-toluenesulfonate and the like; salts of natural amino acids such as glycine, alanine, valine, leucine, isoleucine, norleucine, tyrosine, cystine, cysteine, methionine, proline, hydroxy proline, histidine, omithine, lysine, arginine, and serine; and salts of non-natural amino acids such as D-isomers or substituted amino acids; salts of guanidine; and salts of substituted guanidine wherein the substituents are selected from nitro, amino, alkyl, alkenyl, alkynyl, ammonium or substituted ammonium salts and aluminum salts.

The term “selective inhibitor” as applied to a biologically active agent refers to the agent's ability to selectively reduce the target signaling activity as compared to off-target signaling activity, via direct or indirect interaction with the target.

The term “PI3K-delta selective inhibitor” (also known as PI3K-6 inhibitor) refers to a compound, which selectively inhibits the activity of the PI3K-delta isoform more effectively than other isoforms of the PI3K family (α, β, and γ). In some aspects, the PI3K-delta selective inhibitor refers to a compound of Formula A, as described herein, which selectively inhibits the activity of the PI3K-delta isoform more effectively than other isoforms of the PI3K family (α, β, and γ). For instance, a PI3K-delta selective inhibitor of Formula A can be a compound that exhibits a 50% inhibitory concentration (IC₅₀) with respect to the 6 type PI3-kinase that is at least 20-fold, or lower, than the inhibitor's IC₅₀ with respect to the rest of the other types PI3K isoforms (i.e., α, β, and γ).

The term “Bruton's tyrosine kinase” (also known as “BTK,” agammaglobulinemia tyrosine kinase (ATK), or B-cell progenitor kinase (BPK)) refers to a non-receptor tyrosine kinase enzyme in the B-cell antigen receptor (BCR) signaling pathway. BTK, a member of the Tec family of protein tyrosine kinases, is predominantly expressed in B-lymphocytes at various stages of development (except in terminally differentiated plasma cells). BTK is a signal transduction protein that regulates normal B-cell development, differentiation and functioning, and has also been implicated in initiation, survival, and progression of mature B-cell lymphoproliferative disorders, such as B-cell malignancies. Akinleye, A. et al., J. Hematol. Oncol. 6:59 (2013). As used herein, BTK is from Homo sapiens, as disclosed in U.S. Pat. No. 6,326,469 (Gen Bank Acc. No. NP_000052).

An “inhibitor of BTK” or a “BTK inhibitor” refers to a small molecule that targets BTK and either inhibits BTK tyrosine phosphorylation and/or B-cell activation and/or otherwise inhibits or diminishes or abolishes the biological activity of a BTK protein. An “irreversible BTK inhibitor” refers to a molecule that upon contact with BTK, causes the formation of a new covalent bond with an amino acid residue of BTK. The BTK inhibitor TG-1701, which is used in the methods and kits of the present invention, is an irreversible BTK inhibitor.

The term “synergistic effect,” as used herein, refers to a greater-than-additive therapeutic effect produced by a combination administration of compounds wherein the therapeutic effect obtained with the combination exceeds the additive effects that would otherwise result from individual administration the compounds alone. Aspects of the invention include methods of producing a synergistic effect in the treatment of B-cell malignancies, wherein said effect is at least 5%, at least 10%, at least 20%, at least 30%, at least 40%, at least 50%, at least 60%, at least 70%, at least 80%, at least 90%, at least 100%, at least 200%, at least 500%, or at least 1000% greater than the corresponding additive effect.

“Therapeutic synergy,” as used herein, means that the combined administration of agents, as described herein, (i.e., an anti-CD20 antibody and a PI3K-delta selective inhibitor of Formula A and BTK inhibitor TG-1701) produces a therapeutic effect that is greater than the additive effects of the anti-CD20 antibody, the PI3K-delta selective inhibitor, and the BTK inhibitor, when each is used alone and/or when two agents are combined.

As used in the present disclosure and claims, the singular forms “a,” “an,” and “the” include plural forms unless the context clearly dictates otherwise.

It is understood that wherever aspects are described herein with the language “comprising,” otherwise analogous aspects described in terms of “consisting of” and/or “consisting essentially of” are also provided.

### Methods of Treating B-Cell Malignancies

In one aspect, provided herein are methods of treating a B-cell malignancy in a subject in need thereof, comprising (a) administering to the subject a combination of agents, in therapeutically effective amounts, said combination of agents comprising: (i) a PI3K-delta selective inhibitor; (ii) an anti-CD20 antibody; and (iii) BTK inhibitor TG-1701; and (b) treating said subject with a B-cell malignancy.

In another aspect, provided herein are methods of treating a B-cell malignancy in a subject in need thereof, comprising,

(a) administering to the subject a combination of agents, in therapeutically effective amounts, said combination of agents comprising:
(i) at least one PI3K-delta selective inhibitor of Formula A, or a stereoisomer thereof, or a pharmaceutically acceptable salt, solvate, or prodrug thereof:

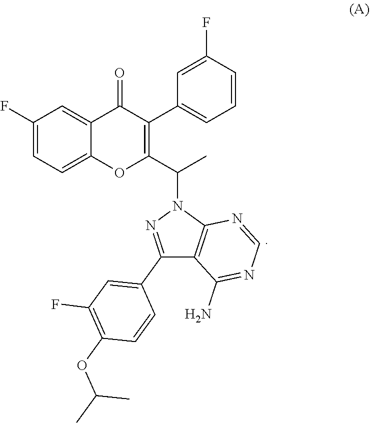 [SMILES: CC(C)Oc1ccc(-c2nn(C(C)c3oc4ccc(F)cc4c(=O)c3-c3cccc(F)c3)c3ncnc(N)c23)cc1F]

selected from one or more of,
(RS)-2-(1-(4-amino-3-(3-fluoro-4-isopropoxyphenyl)-1H-pyrazolo[3,4-d]pyrimidin-1-yl)ethyl)-6-fluoro-3-(3-fluorophenyl)-4H-chromen-4-one;
(S)-2-(1-(4-amino-3-(3-fluoro-4-isopropoxyphenyl)-1H-pyrazolo[3,4-d]pyrimidin-1-yl)ethyl)-6-fluoro-3-(3-fluorophenyl)-4H-chromen-4-one; and
(R)-2-(1-(4-amino-3-(3-fluoro-4-isopropoxyphenyl)-1H-pyrazolo[3,4-d]pyrimidin-1-yl)ethyl)-6-fluoro-3-(3-fluorophenyl)-4H-chromen-4-one;
   (ii) at least one anti-CD20 antibody, wherein at least one anti-CD20 antibody is ublituximab or an anti-CD20 antibody or antibody fragment that binds to the same epitope as ublituximab; and
   (iii) a BTK inhibitor, wherein the BTK inhibitor is (R)-4-amino-1-(1-(but-2-ynoyl)pyrrolidin-3-yl)-3-(4-(2,6-difluorophenoxy)phenyl)-1,6-dihydro-7H-pyrrolo[2,3-d]pyridazin-7-one (TG-1701), or an isomer, polymorph, enantiomer, pharmaceutically acceptable salt, solvate, or prodrug thereof; and

(b) treating said subject with a B-cell malignancy.

In some aspects, the method of treating a B-cell malignancy is effective on subjects whose B-cell malignancy has relapsed.

In some aspects, the B-cell malignancy is selected from the group consisting of acute lymphocytic leukemia (ALL), acute myeloid leukemia (AML), chronic lymphocytic leukemia (CLL), small lymphocytic lymphoma (SLL), multiple myeloma (MM), non-Hodgkin's lymphoma (NHL), mantle cell lymphoma (MCL), follicular lymphoma (FL), Waldenstrom's macroglobulinemia (WM), diffuse large B-cell lymphoma (DLBCL), marginal zone lymphoma (MZL), Burkitt's lymphoma (BL), hairy cell leukemia (HCL), and Richter's transformation.

In some aspects, the B-cell malignancy is CLL.

PI3K-delta Selective Inhibitor

The phosphoinositide 3-kinases (PI3Ks) are a family of enzymes that regulate diverse biological functions in every cell type by generating phosphoinositide second-messenger molecules. PI3Ks are involved in various cellular functions, including cell proliferation and survival, cell differentiation, intracellular trafficking, and immunity. As the activity of these phosphoinositide second messengers is determined by their phosphorylation state, the kinases and phosphatases that act to modify these lipids are central to the correct execution of intracellular signaling events. PI3Ks phosphorylate lipids at the 3-hydroxyl residue of an inositol ring (Whitman et al., Nature 332:664 (1988)) to generate phosphorylated phospholipids (PIP3s), which act as second messengers recruiting kinases with lipid binding domains (including plekstrin homology (PH) regions), such as Akt and phosphoinositide-dependent kinase-1 (PDK1). Binding of Akt to membrane PIP3s causes the translocation of Akt to the plasma membrane, bringing Akt into contact with PDK1, which is responsible for activating Akt. The tumor-suppressor phosphatase PTEN (Phosphatase and Tensin homolog deleted on chromosome Ten) dephosphorylates PIP3 and therefore acts as a negative regulator of Akt activation. The PI3Ks Akt and PDK1 are important in the regulation of many cellular processes including cell cycle regulation, proliferation, survival, apoptosis, and motility and are significant components of the molecular mechanisms of diseases such as cancer, diabetes, and immune inflammation (Vivanco et al., Nature Rev. Cancer 2:489 (2002); Phillips et al., Cancer 83:41 (1998)).

The PI3K family is comprised of four different classes: Classes I, II, III, and IV. Classes I-III are lipid kinases and Class IV are serine/threonine protein kinases.

The members of the Class I family of PI3Ks are dimers of a regulatory and a catalytic subunit. The Class I family consists of four isoforms, determined by the 110 kDa catalytic subunits α, β, γ, and δ. See Engelman, J. A., Nat Rev Genet 7:606-619 (2006); Carnero, A., Curr Cancer Drug Targets 8:187-198 (2008); and Vanhaesebroeck, B., Trends Biochem Sci 30:194-204 (2005). Class I can be subdivided into two subclasses: Class Ia, formed by the combination of p110 α, β, and δ, and a regulatory subunit (p85, p55 or p50); and Class Ib, formed by p110 γ and p101 regulatory subunits. The delta (δ) isoform of PI3K is highly expressed in cells of hematopoietic origin, and strongly upregulated, and often mutated, in various hematologic malignancies.

Studies regarding PI3K and related protein kinase pathways have been published by various groups, including, Liu et al., Nature Reviews Drug Discovery 8:627-644 (2009); Nathan et al, Mol. Cancer Ther. 8(1) (2009); and Marone et al., Biochimica et Biophysica Acta 1784:159-185 (2008). Two known inhibitors of PI3K, LY294002 and Wortmannin, are non-specific PI3K inhibitors as they do not distinguish the four members of Class I PI3K: α, β, γ, and δ. A number of PI3K inhibitors have entered clinical trials for the treatment of cancers, and various types of cancers (including breast cancer, non-small cell lung cancer (NSCLC), and hematological cancers), are being considered as areas of therapeutic interest.

One example of a PI3K-delta selective inhibitor is Idelalisib (trade name Zydelig®), which was approved by the U.S. FDA in 2014 for the treatment of relapsed CLL (in combination with Rituxan®; see, Furman, R. R. et al., N. Eng. J. Med. 370:997-1007 (2014)), relapsed follicular B-cell non-Hodgkin lymphoma (FL), and relapsed small lymphocytic lymphoma (SLL), another type of non-Hodgkin lymphoma. See, Zydelig® full prescribing information (Gilead Sciences). Idelalisib has a unique and limiting toxicity profile including immune mediated colitis (grade 3≥5%), pneumonitis (grade 3≥4%), and transaminitis (grade 3≥8%). Therefore the FDA's approval of Zydelig® comes with a boxed warning noting the possibility of fatal and serious toxicities including hepatic, severe diarrhea, colitis, pneumonitis and intestinal perforation. Id.

Another example of a PI3K-delta selective inhibitor is duvelisib (IPI-145). See, O'Brian, S. et al., Blood 124: Abstract 3334 (2014). Although duvelisib targets both PI3K delta and gamma, at the dose under development (25 mg twice daily), it primarily inhibits just the delta isoform. Id. Another PI3K-delta selective inhibitor is ACP-319 (previously AMG-319). See, Lanasa, M. C. et al., Blood 122: Abstract 678 (2013). ACP-319 is currently in development by Acerta Pharma B.V. ME-401 is a new oral PI3K-delta selective inhibitor in development by MEI Pharma. See, Moreno, O. et al., “Clinical Pharmacokinetics and Pharmacodynamics of ME-401, an Oral, Potent, and Selective Inhibitor of Phosphatidylinositol 3-Kinase P110δ, Following Single Ascending Administration to Healthy Volunteers” (Abstract CT157), presented at the American Association for Cancer Research (AACR) Annual Meeting, New Orleans (Apr. 16-20, 2016). INCB-50465 is another PI3K-delta selective inhibitor in development by Incyte Corporation that is in Phase I/II clinical trials for the treatment of B-cell malignancies. See, Forero-Torres, A. et al., “Preliminary safety, efficacy, and pharmacodynamics of a highly selective PI3Kδ inhibitor, INCB050465, in patients with previously treated B-cell malignancies” (Abstract CT056), presented at the AACR Annual Meeting, New Orleans (Apr. 16-20, 2016).

As provided herein, a PI3K-delta selective inhibitor is used in the methods (and kits) of the present invention. In one embodiment, the PI3K-delta selective inhibitor that is used in combination with the anti-CD20 antibodies and BTK inhibitor TG-1701, described herein, is a compound of formula A:

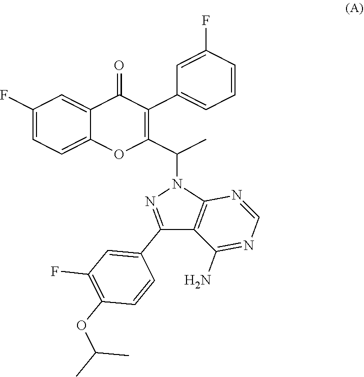 [SMILES: CC(C)Oc1ccc(-c2nn(C(C)c3oc4ccc(F)cc4c(=O)c3-c3cccc(F)c3)c3ncnc(N)c23)cc1F]

or a stereoisomer thereof, or a pharmaceutically acceptable salt, solvate, or prodrug thereof. In a preferred embodiment, the compound of Formula A is selected from one or more of,
(RS)-2-(1-(4-amino-3-(3-fluoro-4-isopropoxyphenyl)-1H-pyrazolo[3,4-d]pyrimidin-1-yl)ethyl)-6-fluoro-3-(3-fluorophenyl)-4H-chromen-4-one;
(S)-2-(1-(4-amino-3-(3-fluoro-4-isopropoxyphenyl)-1H-pyrazolo[3,4-d]pyrimidin-1-yl)ethyl)-6-fluoro-3-(3-fluorophenyl)-4H-chromen-4-one; and
(R)-2-(1-(4-amino-3-(3-fluoro-4-isopropoxyphenyl)-1H-pyrazolo[3,4-d]pyrimidin-1-yl)ethyl)-6-fluoro-3-(3-fluorophenyl)-4H-chromen-4-one.

A PI3K-delta inhibitor of formula A can be prepared using the general synthetic methods as disclosed in International Patent Appl. Publ. No. WO 2011/055215 A2 and U.S. Patent Appl. Publ. No. 2011/0118257 A1.

In some aspects, the PI3K-delta inhibitor is administered to a subject daily at a dosage from: about 200 mg to about 1200 mg, about 400 mg to about 1000 mg, about 400 mg to about 800 mg, about 400 mg, about 500 mg, about 600 mg, about 700 mg, about 800 mg, about 900 mg, about 1000 mg, or about 1200 mg.

In a preferred aspect, the PI3K-delta inhibitor of Formula A is (S)-2-(1-(4-amino-3-(3-fluoro-4-isopropoxyphenyl)-1H-pyrazolo[3,4-d]pyrimidin-1-yl)ethyl)-6-fluoro-3-(3-fluorophenyl)-4H-chromen-4-one, which is also known as umbralisib or TGR-1202. Umbralisib is a free base. The term umbralisib will be used predominantly throughout, although it is to be understood to be interchangeable with TGR-1202.

In some aspects, the PI3K-delta inhibitor of Formula A is (S)-2-(1-(4-amino-3-(3-fluoro-4-isopropoxyphenyl)-1H-pyrazolo[3,4-d]pyrimidin-1-yl)ethyl)-6-fluoro-3-(3-fluorophenyl)-4H-chromen-4-one p-toluenesulfonic acid (PTSA) salt, which is referred to throughout as “umbralisib PTSA salt.” An alternate chemical name for the umbralisib PTSA salt is (S)-2-(1-(4-amino-3-(3-fluoro-4-isopropoxyphenyl)-1H-pyrazolo[3,4-d] pyrimidin-1-yl)-ethyl)-6-fluoro-3-(3-fluorophenyl)-4H-chromen-4-one 4-methylbenzenesulfonate.

Umbralisib (trade name UKONIQg®) was approved by the U.S. FDA in 2021 for the treatment of relapsed or refractory MZL and FL. See, UKONIQ® full prescribing information (TG Therapeutics, Inc.).

The preparation of umbralisib is described in International Publ. No. WO 2014/006572 and U.S. Patent Publ. No. 2014/0011819, each of which is incorporated herein by reference in its entirety. In addition to describing the synthesis of TGR-1202, WO 2014/006572 and US 2014/0011819 also disclose the therapeutic activity of this molecule to inhibit, regulate, and/or modulate the signal transduction of PI3K. TGR-1202 is also described in U.S. Pat. No. 9,150,579, which issued Oct. 6, 2015. International Publ. No. WO 2015/181728, incorporated herein by reference in its entirety, describes a solid state form of umbralisib that exhibits enhanced solubility and pharmacokinetics upon oral administration. The entirety of each of these applications and patents is incorporated herein by reference.

Umbralisib is a PI3K-delta inhibitor with a unique molecular structure and activity profile distinct from other PI3K-delta inhibitors in development, including: (1) greater selectivity to the delta isoform of PI3K; (2) a prolonged half-life that enables once-daily dosing; and (3) a differentiated safety profile from other PI3K-delta inhibitors, notably with respect to hepatic toxicity and colitis.

TGR-1202 (umbralisib) was evaluated in a single-agent Phase I dose-escalation study in patients with relapsed and refractory hematologic malignancies (see e.g., Burris et al., “Activity of TGR-1202, a novel once-daily PI3Kδ inhibitor, in patients with relapsed or refractory hematologic malignancies,” J. Clinical Oncology (ASCO Annual Meeting Abstracts) 32 (15): 2513 (2014)). The study reported that TGR-1202 was well-tolerated in patients with relapsed or refractory hematologic malignancies, with no reported hepatic toxicity and signs of clinical activity at doses≥800 mg each day. Id. The favorable safety profile of TGR-1202 compared to prior inhibitors has also been demonstrated in long-term follow up. See, Burris, H. et al., “Long-term follow-up of the PI3K delta inhibitor TGR-1202 demonstrates a differentiated safety profile and high response rates in CLL and NHL: Integrated-analysis of TGR-1202 monotherapy and combined with ublituximab,” American Society of Clinical Oncology Annual Meeting (ASCO), Abstract 7512 (Jun. 3, 2016).

In some aspects, umbralisib is administered at a dose from about 400 mg to about 1200 mg per day. In some aspects, the TGR-1202 is administered at a dose of about 400 mg per day. In some aspects, the TGR-1202 is administered at a dose of about 600 mg per day. In some aspects, the TGR-1202 is administered at a dose of about 800 mg per day.

In some aspects, the PI3K-delta inhibitor is formulated for oral administration. In certain aspects, the PI3K-delta inhibitor is TGR-1202 and it is formulated for daily oral administration. In certain aspects, TGR-1202 is administered in a fed-state.

### Anti-CD20 Antibodies

CD20 is a hydrophobic transmembrane phosphoprotein that is expressed predominantly in pre-B cells and mature peripheral B cells in humans and mice. In humans, CD20 is also strongly and homogeneously expressed in most mature B-cell malignancies, including, for example, most non-Hodgkin's B-cell lymphomas (NHL) and B-type Chronic Lymphocytic Leukemia's (B-CLL). The CD20 antigen is not expressed on haematopoietic stem cells or on plasmocytes.

Anti-CD20 monoclonal antibodies have been, and continue to be, developed for the treatment of B-cell diseases. The chimeric anti-CD20 monoclonal antibody rituximab (Rituxan®) has become the standard therapy for many CD20-positive B-cell lymphomas and was the first mAb approved for any oncology indication. Demarest, S J et al., mAbs 3:338-351 (2011). However, there are a substantial number of patients who are refractory to treatment with rituximab or who develop resistance in the course of prolonged treatment with rituximab (used as a single agent or even in combination with chemotherapeutic regimens).

As provided herein, anti-CD20 antibodies and antigen-binding fragments thereof can be used in combination with a PI3K-delta selective inhibitor and a BTK inhibitor to treat B-cell proliferative disorders, such as B-cell malignancies. More than one anti-CD20 antibody can be used in the methods and kits of the present invention.

Aside from rituximab, a number of other anti-CD20 antibodies are known in the art, including, for example, ublituximab, ofatumumab (HuMax; Intracel), ocrelizumab, veltuzumab, GA101 (obinutuzumab), AME-133v (Applied Molecular Evolution), ocaratuzumab (Mentrik Biotech), PRO131921, tositumomab, ibritumomab-tiuxetan, hA20 (Immunomedics, Inc.), BLX-301 (Biolex Therapeutics), Reditux (Dr. Reddy's Laboratories), and PRO70769 (described in WO2004/056312).

Rituximab is a genetically engineered chimeric murine/human monoclonal antibody directed against the CD20 antigen. Rituximab is the antibody called “C2B8” in U.S. Pat. No. 5,736,137. The amino acid sequence of rituximab antibody and exemplary methods for its production via recombinant expression in Chinese Hamster Ovary (CHO) cells are disclosed in U.S. Pat. No. 5,736,137, which is herein incorporated by reference in its entirety. Rituximab is commercially available as Rituxan®.

Ofatumumab is an anti-CD20 IgG1κ human monoclonal antibody. Studies indicate that ofatumumab dissociates from CD20 at a slower rate compared to the rituximab and binds a membrane-proximal epitope. Zhang et al., Mabs 1: 326-331 (2009). Epitope mapping has indicated that ofatumumab binds an epitope located closer to the N-terminus of CD20 compared to the location targeted by rituximab and includes an extracellular loop of the antigen. Id.

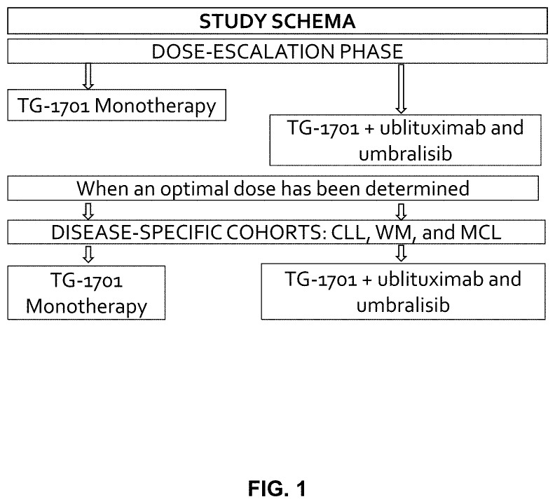

Ublituximab (also known as UTX, TG-1101, TGTX-1101, Utuxin™, LFB-R603, TG20, EMAB603) is a chimeric monoclonal antibody targeting a unique epitope on the CD20 antigen (see, e.g., FIG. 1 in Fox, E. et al., Mult. Scler. 27(3): 420-429 (March 2021), and that has been glycoengineered for enhanced affinity for all variants of Fcγ RIIIa receptors, thereby demonstrating greater antibody-dependent cellular cytotoxicity (“ADCC”) activity than rituximab and ofatumab. See, Miller, J. et al., Blood 120: Abstract No. 2756 (2012); Deng, C. et. al., J. Clin. Oncol. 31: Abstract No. 8575 (2013); O'Connor, O. A. et al., J. Clin. Oncol. 32:5s (2014), (suppl: Abstract No. 8524). Ublituximab is also described in U.S. Pat. No. 9,234,045. Ublituximab was engineered for potent activity, exhibiting a unique primary amino acid sequence and allowing a low fucose content, designed to induce superior ADCC. Responses with single agent ublituximab were observed in rituximab refractory patients. Id.

In some aspects, the anti-CD20 antibody used in the methods (and kits) described herein is ublituximab, or an anti-CD20 antibody that binds to the same epitope as ublituximab. In certain aspects, the anti-CD20 antibody is ublituximab. In some aspects, the ublituximab comprises the VH CDR1, CDR2, and CDR3 region of sequences SEQ ID NOS: 1, 2, and 3, and the VL CDR1, CDR2, and CDR3 region of sequences SEQ ID NOS: 6, 7, and 8. In some aspects, the ublituximab comprises the VH of SEQ ID NO: 4 and the VL of SEQ ID NO: 9.

Ublituximab comprises the antibody sequences provided below:

Variable heavy chain (VH) CDR1:
(SEQ ID NO: 1)
Gly Tyr Thr Phe Thr Ser Tyr Asn
Variable heavy chain (VH) CDR2:
(SEQ ID NO: 2)
Ile Tyr Pro Gly Asn Gly Asp Thr
Variable heavy chain (VH) CDR3:
(SEQ ID NO: 3)
Ala Arg Tyr Asp Tyr Asn Tyr Ala Met Asp Tyr
Variable heavy chain (VH):
(SEQ ID NO: 4)
Gln Ala Tyr Leu Gln Ser Gly Ala Glu Leu Val Arg
Pro Gly Ala Ser Val Lys Met Ser Cys Lys Ala Ser
Gly Tyr Thr Phe Thr Ser Tyr Asn Met His Trp Val
Lys Gln Thr Pro Arg Gln Gly Leu Glu Trp Ile Gly
Ile Tyr Pro Gly Asn Gly Asp Thr Ser Tyr Asn Gln
Lys Phe Lys Gly Lys Ala Thr Leu Thr Val Gly Lys
Ser Thr Ala Tyr Met Gln Leu Ser Leu Thr Ser Glu
Asp Ser Ala Val Tyr Phe Cys Ala Arg Tyr Asp Tyr
Asn Tyr Ala Met Asp Tyr Trp Gly Gln Gly Thr Ser
Val Thr Val Ser
Constant heavy chain:
(SEQ ID NO: 5)
Ala Ser Thr Lys Gly Pro Ser Val Phe Pro Leu Ala
Pro Ser Lys Ser Thr Ser Gly Thr Ala Leu Gly Cys
Leu Val Lys Asp Tyr Phe Pro Glu Pro Val Thr Val
Ser Trp Asn Ser Gly Ala Leu Thr Ser Gly Val His
Thr Phe Pro Ala Val Leu Gln Ser Gly Leu Tyr Ser
Leu Ser Val Thr Val Pro Ser Ser Ser Leu Gly Thr
Gln Thr Tyr Ile Cys Asn Val Asn His Lys Pro Ser
Asn Thr Lys Val Asp Lys Lys Val Glu Pro Lys Ser
Cys Asp Lys Thr His Thr Cys Pro Pro Cys Pro Ala
Pro Glu Leu Leu Gly Gly Pro Ser Val Phe Leu Phe
Pro Pro Lys Pro Lys Asp Thr Leu Met Ile Ser Arg
Thr Pro Glu Val Thr Cys Val Val Val Asp Val Ser
His Glu Asp Pro Glu Val Lys Phe Asn Trp Tyr Val
Asp Gly Val Glu Val His Asn Ala Lys Thr Lys Pro
Arg Glu Glu Gln Tyr Asn Ser Thr Tyr Arg Val Val
Ser Val Leu Thr Val Leu His Gln Asp Trp Leu Asn
Gly Lys Glu Tyr Lys Cys Lys Val Ser Asn Lys Ala
Leu Pro Ala Pro Ile Glu Lys Thr Ile Ser Lys Ala
Lys Gly Gln Pro Arg Glu Pro Gln Val Tyr Thr Leu
Pro Pro Ser Arg Asp Glu Leu Thr Lys Asn Gln Val
Ser Leu Thr Cys Leu Val Lys Gly Phe Tyr Pro Ser
Asp Ile Ala Val Glu Trp Glu Ser Asn Gly Gln Pro
Glu Asn Asn Tyr Lys Thr Thr Pro Pro Val Leu Asp
Ser Asp Gly Ser Phe Phe Leu Tyr Ser Lys Leu Thr
Val Asp Lys Ser Arg Trp Gln Gln Gly Asn Val Phe
Ser Cys Ser Val Met His Glu Ala Leu His Asn His
Tyr Thr Gln Lys Ser Leu Ser Leu Ser Pro Gly Lys
Variable light chain (VL) CDR1:
(SEQ ID NO: 6)
Ser Ser Val Ser Tyr
Variable light chain (VL) CDR2:
(SEQ ID NO: 7)
Ala Thr Ser
Variable light chain (VL) CDR3:
(SEQ ID NO: 8)
Gln Gln Trp Thr Phe Asn Pro Pro Thr
Variable light chain (VL):
(SEQ ID NO: 9)
Gln Ile Val Leu Ser Gln Ser Pro Ala Ile Leu Ser
Ala Ser Pro Gly Glu Lys Val Thr Met Thr Cys Arg
Ala Ser Ser Ser Val Ser Tyr Met His Trp Tyr Gln
Gln Lys Pro Gly Ser Ser Pro Lys Pro Trp Ile Tyr
Ala Thr Ser Asn Leu Ala Ser Gly Val Pro Ala Arg
Phe Ser Gly Ser Gly Ser Gly Thr Ser Tyr Ser Phe
Thr Ile Ser Arg Val Glu Ala Glu Asp Ala Ala Thr
Tyr Tyr Cys Gln Gln Trp Thr Phe Asn Pro Pro Thr
Phe Gly Gly Gly Thr Arg Leu Glu Ile Lys
Constant light chain:
(SEQ ID NO: 10)
Thr Val Ala Ala Pro Ser Val Phe Ile Phe Pro Pro
Ser Asp Glu Gln Leu Lys Ser Gly Thr Ala Ser Val
Val Cys Leu Leu Asn Asn Phe Tyr Pro Arg Glu Ala
Lys Val Gln Trp Lys Val Asp Asn Ala Leu Gln Ser
Gly Asn Ser Gln Glu Ser Val Thr Glu Gln Asp Ser
Lys Asp Ser Thr Tyr Ser Leu Ser Ser Thr Leu Thr
Leu Ser Lys Ala Asp Tyr Glu Lys His Lys Val Tyr
Ala Cys Glu Val Thr His Gln Gly Leu Ser Ser Pro
Val Thr Lys Ser Phe Asn Arg Gly Glu Cys

In some aspects, ublituximab is administered at a dose from: about 450 mg to about 1200 mg, about 600 to about 1200 mg, about 600 to about 1000 mg, about 600 to about 900 mg, about 600 mg, about 700 mg, about 800 mg, or about 900 mg.

In certain aspects, the ublituximab is administered at a dose of about 900 mg.

In some aspects, ublituximab may be administered about once every 1 to 9 weeks, about once every week, about twice every week, about once every 2 weeks, about once every 3 weeks, about once every 4 weeks, about once every 5 weeks, about once every 6 weeks, about once every 7 weeks, about once every 8 week, or about once every 9 weeks. One skilled in the art will appreciate that the dosage of ublituximab and/or frequency of administering ublituximab may change during the course of therapy (lowered or increased) depending upon the patient's clinical response, side effects, etc. . . . .

In certain aspects, ublituximab is administered on days 1, 8, and 15 of cycle 1 and day 1 of cycles 2, 3, 4, 5, 6, and every 3 months thereafter, wherein each cycle is about 28 days. In this dosing regimen, ublituximab is administered at a dose of about 900 mg. In certain aspects, each cycle is 28 days.

In some aspects, the ublituximab is administered intravenously, preferably by infusion.

In some aspects, the anti-CD20 antibody or fragment thereof binds to the same epitope as ublituximab. In some aspects, the anti-CD20 antibody or fragment thereof binds to a sequence comprising amino acids N153-S179 of CD20. In some aspects, the anti-CD20 antibody or fragment thereof binds to a discontinuous epitope in amino acids N153-S179 of CD20.

In some aspects, the anti-CD20 antibody or fragment thereof binds to CD20 with an affinity characterized by a dissociation constant KD of less than about 10⁻⁷ M, less than about 10⁻⁸ M or less than about 10⁻⁹ M. In some aspects, the anti-CD20 antibody or fragment thereof binds to CD20 with an affinity characterized by a dissociation constant KD of 10⁻¹⁰ to 10⁻⁹ M. In some aspects the anti-CD20 antibody or fragment thereof binds to CD20 with an affinity characterized by a dissociation constant KD of 0.7×10⁻⁹ M. As used in the context of antibody binding dissociation constants, the term “about” allows for the degree of variation inherent in the methods utilized for measuring antibody affinity. For example, depending on the level of precision of the instrumentation used, standard error based on the number of samples measured, and rounding error, the term “about 10-2 M” might include, for example, from 0.05 M to 0.005 M.

In some aspects, the anti-CD20 antibody exhibits a high affinity to Fc-gammaRIII (CD16). In some aspects, as a result of their high affinity for the Fc region of the antibody to CD16, such antibodies are not displaced by IgG polyclonal antibodies, especially by IgG present in blood serum. In some aspects the antibody binds to CD16 (e.g., expressed on a macrophage) with an affinity of at least 2×10⁶ M⁻¹, at least 2×10⁷M⁻¹, 2×10⁸M⁻¹ or 2×10⁷ M⁻¹, e.g., as determined by Scatchard analysis or BIAcore technology (Label-free surface plasmon resonance based technology).

In some aspects, the anti-CD20 antibody is glycoengineered. As used herein, a “glycoengineered” anti-CD20 antibody means that the sugar molecules (N-glycan) in the Fc region of the antibody have been altered or engineered, either genetically, enzymatically, chemically, or selected for during the manufacturing process. in order to, e.g., increase the affinity of the antibody for Fc receptors on effector cells and/or to reduce its specific carbohydrate content in its Fc region.

In some aspects, the anti-CD20 antibody exhibits a glycosylation pattern characterized by low fucose content in its Fc region. For example, in some aspects, a composition comprises anti-CD20 antibodies in which the antibodies comprise N-glycoside-linked sugar chains bound on the Fc-gamma glycosylation site (Asn 297, EU numbering), wherein among the N-glycoside-linked sugar chains of all the antibodies of the composition, the fucose content is less than 65%, less than 60%, less than 55%, less than 50%, less than 45%, or less than 40%. In some aspects, among the N-glycoside-linked sugar chains of all the antibodies of the composition, the fucose content is 15 to 45% or 20 to 40%.

In some aspects, the anti-CD20 antibody exhibits potent in vitro antibody-dependent cellular cytotoxicity (ADCC) and can be said to be “ADCC-optimized.” In some aspects, the anti-CD20 antibody produces an ADCC plateau of at least about 10%, at least about 15%, at least about 20%, at least about 25%, or at least about 30% at a concentration of 50 ng/ml using natural killer (NK) cells from healthy donors. Techniques for measuring ADCC are known in the art and provided, for example, in de Romeuf, C. et al., British Journal of Haematology 140: 635-643 (2008). In some aspects, the anti-CD20 antibody produces an ADCC plateau at about 35% at a concentration of 50 ng/ml using NK cells from healthy donors.

In some aspects, the anti-CD20 antibody can decrease NF-kappa-B activity. In some aspects, the anti-CD20 antibody can decrease SNAIL expression. In some aspects, the anti-CD20 antibody can increase RKIP activity. In some aspects, the anti-CD20 antibody can increase PTEN activity. In some aspects, the anti-CD20 antibody can increase sensitization of a cell to TRAIL-apoptosis.

In some aspects, the anti-CD20 antibody is Fc-gamma-RIIIA (CD16) optimized. Antibodies capable of activating type III Fc receptors and having a particular glycan structure have been described, for example, in U.S. Pat. No. 7,931,895, which is herein incorporated by reference in its entirety. Thus, in some aspects, the anti-CD20 antibody is modified on Asn 297 (EU numbering) with N-glycosylations of the bi-antennary and/or oligomannoside type as described in U.S. Pat. No. 7,931,895. Methods of producing antibodies with strong affinity for receptor CD16 of the effector cells of the immune system are provided, for example, in U.S. Published Appl. No. 2005/0271652, which is herein incorporated by reference in its entirety.

In some aspects, the anti-CD20 antibody has high ADCC activity. Methods of producing antibodies with high ADCC activity are provided, for example, in U.S. Pat. No. 7,713,524, which is herein incorporated by reference in its entirety.

Thus, in some aspects, an isolated antibody or antigen-binding fragment, variant, or derivative thereof comprises, consists essentially of, or consists of an immunoglobulin heavy chain variable domain (VH domain), wherein at least one (i.e., one, two, or three) of the CDRs of the VH domain has an amino acid sequence that is at least 80%, 85%, 90%, 95%, 96%, 97%, 98%, 99%, or identical to the CDR1, CDR2, or CDR3 region of sequences SEQ ID NO:1, 2, or 3, wherein an antibody or antigen-binding fragment thereof comprising the VH domain can specifically or preferentially bind to CD20.

In some aspects, an isolated antibody or antigen-binding fragment, variant, or derivative thereof comprises, consists essentially of, or consists of an immunoglobulin heavy chain variable domain (VH domain), wherein at least one (i.e., one, two, or three) of the CDRs of the VH domain has an amino acid sequence identical, except for 1, 2, 3, 4, or 5 conservative amino acid substitutions, to the CDR1, CDR2, or CDR3 region of sequences SEQ ID NO:1, 2, or 3, wherein an antibody or antigen-binding fragment, variant, or derivative thereof comprising the VH domain can specifically or preferentially bind to CD20.

In some aspects, an isolated antibody or antigen-binding fragment, variant, or derivative thereof comprises, consists essentially of, or consists of a VH domain that has an amino acid sequence that is at least 80%, 85%, 90%, 91%, 92%, 93%, 94%, 95%, 96%, 97%, 98%, 99%, or 100% identical to a VH amino acid sequence of SEQ ID NO:4, wherein an antibody or antigen-binding fragment, variant, or derivative thereof comprising the VH domain can specifically or preferentially bind to CD20.

In some aspects, an isolated antibody or antigen-binding fragment, variant, or derivative thereof comprises, consists essentially of, or consists of a heavy chain that has an amino acid sequence that is at least 80%, 85%, 90%, 91%, 92%, 93%, 94%, 95%, 96%, 97%, 98%, 99%, or 100% identical to a heavy chain amino acid sequence comprising SEQ ID NOs: 4 and 5, wherein an antibody or antigen-binding fragment, variant, or derivative thereof comprising the heavy chain can specifically or preferentially bind to CD20.

In some aspects, an isolated antibody or antigen-binding fragment, variant, or derivative thereof comprises, consists essentially of, or consists of an immunoglobulin light chain variable domain (VL domain), wherein at least one (i.e., one, two, or three) of the CDRs of the VL domain has an amino acid sequence that is at least 80%, 85%, 90%, 95%, 96%, 97%, 98%, 99%, or identical to the CDR1, CDR2, or CDR3 region of sequences SEQ ID NO:6, 7, or 8, wherein an antibody or antigen-binding fragment thereof comprising the VL domain can specifically or preferentially bind to CD20.

In some aspects, an isolated antibody or antigen-binding fragment, variant, or derivative thereof comprises, consists essentially of, or consists of an immunoglobulin light chain variable domain (VL domain), wherein at least one (i.e., one, two, or three) of the CDRs of the VL domain has an amino acid sequence identical, except for 1, 2, 3, 4, or 5 conservative amino acid substitutions, to the CDR1, CDR2, or CDR3 region of SEQ ID NO:6, 7, or 8, wherein an antibody or antigen-binding fragment, variant, or derivative thereof comprising the VL domain can specifically or preferentially bind to CD20.

In some aspects, an isolated antibody or antigen-binding fragment, variant, or derivative thereof comprises, consists essentially of, or consists of a VL domain that has an amino acid sequence that is at least 80%, 85%, 90%, 91%, 92%, 93%, 94%, 95%, 96%, 97%, 98%, 99%, or 100% identical to a VL amino acid sequence of SEQ ID NO:9, wherein an antibody or antigen-binding fragment, variant, or derivative thereof comprising the VL domain can specifically or preferentially bind to CD20.

In some aspects, an isolated antibody or antigen-binding fragment, variant, or derivative thereof comprises, consists essentially of, or consists of a light chain that has an amino acid sequence that is at least 80%, 85%, 90%, 91%, 92%, 93%, 94%, 95%, 96%, 97%, 98%, 99%, or 100% identical to a light chain amino acid sequence comprising SEQ ID NOs:9 and 10, wherein an antibody or antigen-binding fragment, variant, or derivative thereof comprising the light chain can specifically or preferentially bind to CD20.

In some aspects, an isolated antibody or antigen-binding fragment, variant, or derivative thereof comprises, consists essentially of, or consists of an immunoglobulin heavy chain variable domain (VH domain) and an immunoglobulin light chain variable domain (VL domain), wherein at least one (i.e., one, two, or three) of the CDRs of the VH domain has an amino acid sequence that is at least 80%, 85%, 90%, 95%, 96%, 97%, 98%, 99%, or identical to the CDR1, CDR2, or CDR3 region of sequences SEQ ID NO:1, 2, or 3, wherein at least one (i.e., one, two, or three) of the CDRs of the VL domain has an amino acid sequence that is at least 80%, 85%, 90%, 95%, 96%, 97%, 98%, 99%, or identical to the CDR1, CDR2 or CDR3 region of sequences SEQ ID NO:6, 7, or 8, and wherein an antibody or antigen-binding fragment thereof comprising the VH domain and VL domain can specifically or preferentially bind to CD20.

In some aspects, an isolated antibody or antigen-binding fragment, variant, or derivative thereof comprises, consists essentially of, or consists of an immunoglobulin heavy chain variable domain (VH domain), and an immunoglobulin light chain variable domain (VL domain), wherein at least one (i.e., one, two, or three) of the CDRs of the VH domain has an amino acid sequence identical, except for 1, 2, 3, 4, or 5 conservative amino acid substitutions, to the CDR1, CDR2, or CDR3 region of sequences SEQ ID NO:1, 2, or 3, wherein at least one (i.e., one, two, or three) of the CDRs of the VL domain has an amino acid sequence identical, except for 1, 2, 3, 4, or 5 conservative amino acid substitutions, to the CDR1, CDR2 or CDR3 region of SEQ ID NO:6, 7, or 8, and wherein an antibody or antigen-binding fragment, variant, or derivative thereof comprising the VH and VL can specifically or preferentially bind to CD20.

In some aspects, the anti-CD20 antibody or antigen-binding fragment, variant, or derivative thereof comprises the VH CDR1, CDR2, and CDR3 region of sequences SEQ ID NO:1, 2, and 3, and the VL CDR1, CDR2, and CDR3 region of sequences SEQ ID NO:6, 7, and 8.

In some aspects, an isolated antibody or antigen-binding fragment, variant, or derivative thereof comprises, consists essentially of, or consists of a VH domain and a VL domain, wherein the VH has an amino acid sequence that is at least 80%, 85%, 90%, 91%, 92%, 93%, 94%, 95%, 96%, 97%, 98%, 99%, or 100% identical to a VH amino acid sequence of SEQ ID NO:4, wherein the VL domain that has an amino acid sequence that is at least 80%, 85%, 90%, 91%, 92%, 93%, 94%, 95%, 96%, 97%, 98%, 99%, or 100% identical to a VL amino acid sequence of SEQ ID NO:9, and wherein an antibody or antigen-binding fragment, variant, or derivative thereof comprising the VH domain and VL domain can specifically or preferentially bind to CD20.

In some aspects, the anti-CD20 antibody or antigen-binding fragment thereof comprises the VH of SEQ ID NO:4 and the VL of SEQ ID NO:9.

In some aspects, the anti-CD20 antibody or antigen-binding fragment thereof binds to the same epitope as an antibody comprising the VH of SEQ ID NO:4 and the VL of SEQ ID NO:9.

In some aspects, an isolated antibody or antigen-binding fragment, variant, or derivative thereof comprises, consists essentially of, or consists of a heavy chain and a light chain, wherein the heavy chain has an amino acid sequence that is at least 80%, 85%, 90%, 91%, 92%, 93%, 94%, 95%, 96%, 97%, 98%, 99%, or 100% identical to a heavy chain amino acid sequence comprising SEQ ID NOs: 4 and 5, wherein the light chain has an amino acid sequence that is at least 80%, 85%, 90%, 91%, 92%, 93%, 94%, 95%, 96%, 97%, 98%, 99%, or 100% identical to a light chain amino acid sequence comprising SEQ ID NOs: 9 and 10, and wherein an antibody or antigen-binding fragment, variant, or derivative thereof comprising the heavy chain and light chain can specifically or preferentially bind to CD20.

In some aspects, the anti-CD20 antibody or antigen-binding fragment thereof comprises a heavy chain comprising SEQ ID NOs: 4 and 5 and a light chain comprising SEQ ID NOs: 9 and 10.

In some aspects, the anti-CD20 antibody or antigen-binding fragment thereof binds to the same epitope as an antibody comprising SEQ ID NO:4 and SEQ ID NO:5.

In some aspects, the anti-CD20 antibody is ublituximab.

In some aspects, the anti-CD20 antibody is EMAB603 (see WO2006/064121, which is herein incorporated by reference in its entirety), produced by the clone R603-12D11, and deposited to the Collection Nationale des Cultures de Microorganismes under the accession number CNCM 1-3529.

In some aspects, the anti-CD20 antibody is produced in the rat hybridoma YB2/0 cell line (cell YB2/3HL.P2.G11.16Ag.20, registered at the American Type Culture Collection under ATCC number CRL-1662).

The precise chemical structure of an antibody capable of specifically binding CD20 and retaining the desired activity depends on a number of factors. As ionizable amino and carboxyl groups are present in the molecule, a particular polypeptide can be obtained as an acidic or basic salt, or in neutral form. All such preparations that retain their biological activity when placed in suitable environmental conditions are included in the definition of anti-CD20 antibodies as used herein. Further, the primary amino acid sequence of the antibody can be augmented by derivatization using sugar moieties (glycosylation) or by other supplementary molecules such as lipids, phosphate, acetyl groups and the like. It can also be augmented by conjugation with saccharides. Certain aspects of such augmentation are accomplished through post-translational processing systems of the producing host; other such modifications can be introduced in vitro. In any event, such modifications are included in the definition of an anti-CD20 antibody used herein so long as the desired properties of the anti-CD20 antibody are not destroyed. It is expected that such modifications can quantitatively or qualitatively affect the activity, either by enhancing or diminishing the activity of the polypeptide, in the various assays. Further, individual amino acid residues in the chain can be modified by oxidation, reduction, or other derivatization, and the polypeptide can be cleaved to obtain fragments that retain activity. Such alterations that do not destroy the desired properties (e.g., binding specificity for CD20) do not remove the polypeptide sequence from the definition of anti-CD20 antibodies of interest as used herein.

The art provides substantial guidance regarding the preparation and use of polypeptide variants. In preparing variants of an anti-CD20 binding molecule, e.g., an antibody or antigen-binding fragment, variant, or derivative thereof, one of skill in the art can readily determine which modifications to the native protein's nucleotide or amino acid sequence will result in a variant that is suitable for use as a therapeutically active component of a pharmaceutical composition.

It is possible to introduce mutations only in framework regions or only in CDR regions of an antibody molecule. Introduced mutations can be silent or neutral missense mutations, i.e., have no, or little, effect on an antibody's ability to bind antigen. These types of mutations can be useful to optimize codon usage, or improve a hybridoma's antibody production. Alternatively, non-neutral missense mutations can alter an antibody's ability to bind antigen. The location of most silent and neutral missense mutations is likely to be in the framework regions, while the location of most non-neutral missense mutations is likely to be in CDR, though this is not an absolute requirement. One of skill in the art would be able to design and test mutant molecules with desired properties such as no alteration in antigen-binding activity or alteration in binding activity (e.g., improvements in antigen-binding activity or change in antibody specificity). Following mutagenesis, the encoded protein can routinely be expressed and the functional and/or biological activity of the encoded protein, (e.g., ability to immunospecifically bind at least one epitope of a CD20 polypeptide) can be determined using techniques described herein or by routinely modifying techniques known in the art.

In certain aspects, the anti-CD20 antibodies comprise at least one optimized complementarity-determining region (CDR). By “optimized CDR” is intended that the CDR has been modified and optimized sequences selected based on the sustained or improved binding affinity and/or anti-CD20 activity that is imparted to an anti-CD20 antibody comprising the optimized CDR. “Anti-CD20 activity” can include, e.g., activity which modulates one or more of the following activities associated with CD20, e.g., the ability to induce apoptosis of B-cells, the ability to induce ADCC against B-cells (e.g., CLL cells), the ability to inhibit NF-kappaB activity, the ability to inhibit Snail expression, the ability to de-repress RKIP, the ability to de-repress PTEN, the ability to sensitize a tumor cell to TRAIL-apoptosis or any other activity associated with CD20. Such activities are described, for example, in Baritaki, S. et al., Int. J. Oncol. 38: 1683-1694 (2011), which is herein incorporated by reference in its entirety. The modifications can involve replacement of amino acid residues within the CDR such that an anti-CD20 antibody retains specificity for the CD20 antigen and has improved binding affinity and/or improved anti-CD20 activity.

In certain anti-CD20 antibodies, or antigen-binding fragments thereof, at least a fraction of one or more of the constant region domains has been deleted or otherwise altered so as to provide desired biochemical characteristics such as reduced effector functions, the ability to non-covalently dimerize, increased ability to localize at the site of a tumor, reduced serum half-life, or increased serum half-life when compared with a whole, unaltered antibody of approximately the same immunogenicity. For example, certain antibodies are domain deleted antibodies which comprise a polypeptide chain similar to an immunoglobulin heavy chain, but which lack at least a portion of one or more heavy chain domains. For instance, in certain antibodies, one entire domain of the constant region of the modified antibody will be deleted, for example, all or part of the CH2 domain will be deleted.

In certain anti-CD20 antibodies or antigen-binding fragments thereof, the Fc portion can be mutated to decrease effector function using techniques known in the art. For example, modifications of the constant region can be used to modify disulfide linkages or oligosaccharide moieties that allow for enhanced localization due to increased antigen specificity or antibody flexibility. The resulting physiological profile, bioavailability and other biochemical effects of the modifications can easily be measured and quantified using well know immunological techniques without undue experimentation.

In certain aspects, an anti-CD20 antibody or antigen-binding fragment thereof will not elicit a deleterious immune response in the animal to be treated, e.g., in a human. In one embodiment, anti-CD20 antibodies or antigen-binding fragments thereof can be modified to reduce their immunogenicity using art-recognized techniques. For example, antibodies can be humanized, primatized, deimmunized, or chimeric antibodies can be made. These types of antibodies are derived from a non-human antibody, typically a murine or primate antibody, that retains or substantially retains the antigen-binding properties of the parent antibody, but which is less immunogenic in humans. This can be achieved by various methods, including (a) grafting the entire non-human variable domains onto human constant regions to generate chimeric antibodies; (b) grafting at least a part of one or more of the non-human complementarity determining regions (CDRs) into a human framework and constant regions with or without retention of critical framework residues; or (c) transplanting the entire non-human variable domains, but “cloaking” them with a human-like section by replacement of surface residues. Such methods are disclosed in Morrison et al., Proc. Natl. Acad. Sci. 81:6851-6855 (1984); Morrison et al., Adv. Immunol. 44:65-92 (1988); Verhoeyen et al., Science 239:1534-1536 (1988); Padlan, Molec. Immun. 28:489-498 (1991); Padlan, Molec. Immun. 31:169-217 (1994), and U.S. Pat. Nos. 5,585,089, 5,693,761, 5,693,762, and 6,190,370, all of which are hereby incorporated by reference in their entirety.

Modified forms of antibodies or antigen-binding fragments thereof can be made from whole precursor or parent antibodies using techniques known in the art.

Anti-CD20 antibodies or antigen-binding fragments thereof can be made or manufactured using techniques that are known in the art. In certain aspects, antibody molecules or fragments thereof are “recombinantly produced,” i.e., are produced using recombinant DNA technology. Anti-CD20 antibodies or fragments thereof can be generated by any suitable method known in the art including generation of polyclonal antibodies or preparation of monoclonal antibodies, e.g., through hybridoma or phage display.

A variety of host-expression vector systems can be utilized to express antibody molecules. The host cell can be co-transfected with two expression vectors, the first vector encoding a heavy chain derived polypeptide and the second vector encoding a light chain derived polypeptide. The two vectors can contain identical selectable markers which enable equal expression of heavy and light chain polypeptides. Alternatively, a single vector can be used which encodes both heavy and light chain polypeptides. In such situations, the light chain is advantageously placed before the heavy chain to avoid an excess of toxic free heavy chain (Proudfoot, Nature 322:52 (1986); Kohler, Proc. Natl. Acad. Sci. USA 77:2197 (1980)). The host cell can also be transfected with a single vector encoding a heavy chain derived polypeptide and a light chain derived polypeptide. The coding sequences for the heavy and light chains can comprise cDNA or genomic DNA.

The expression vector or vectors can be transferred to a host cell by conventional techniques and the transfected cells are then cultured by conventional techniques to produce an antibody. Thus, host cells containing a polynucleotide encoding an antibody, or a heavy or light chain thereof, operably linked to a heterologous promoter are provided. In certain aspects for the expression of double-chained antibodies, vectors encoding both the heavy and light chains can be co-expressed in the host cell for expression of the entire immunoglobulin molecule.

Host-expression systems represent vehicles by which the coding sequences of interest can be produced and subsequently purified, but also represent cells which can, when transformed or transfected with the appropriate nucleotide coding sequences, express a CD20 antibody in situ. These include but are not limited to microorganisms such as bacteria (e.g., E. coli, B. subtilis) transformed with recombinant bacteriophage DNA, plasmid DNA or cosmid DNA expression vectors containing antibody coding sequences; yeast (e.g., Saccharomyces, Pichia) transformed with recombinant yeast expression vectors containing antibody coding sequences; insect cell systems infected with recombinant virus expression vectors (e.g., baculovirus) containing antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BLK, 293, 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Bacterial cells such as Escherichia coli, or eukaryotic cells, e.g., for the expression of whole recombinant antibody molecules, are used for the expression of a recombinant antibody molecule. For example, mammalian cells such as Chinese hamster ovary cells (CHO), in conjunction with a vector such as the major intermediate early gene promoter element from human cytomegalovirus is an effective expression system for antibodies (Foecking et al., Gene 45:101 (1986); Cockett et al., Bio/Technology 8:2 (1990)). In some aspects, the anti-CD20 antibody is produced in a host cell that is not a CHO cell.

Once an antibody has been recombinantly expressed, it can be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins.

In some aspects, the anti-CD20 antibody is produced by a rat hybridoma cell line. In some aspects, the anti-CD20 antibody is produced in YB2/0 (ATCC CRL-1662)

In some aspects, the anti-CD20 antibody is ublituximab and it is administered to the subject at a dose from: about 450 mg to about 1200 mg, about 600 to about 1200 mg, about 600 to about 1000 mg, about 600 to about 900 mg, about 600 mg, about 700 mg, about 800 mg, or about 900 mg about twice every week, about once every 1 to 9 weeks, about once every week, about twice every week, about once every 2 weeks, about once every 3 weeks, about once every 4 weeks, about once every 5 weeks, about once every 6 weeks, about once every 7 weeks, about once every 8 week, or about once every 9 weeks.

In certain aspects, ublituximab is administered at a dose of about 900 mg about once every 1 to 9 weeks. In certain aspects, ublituximab is administered intravenously.

### BTK Inhibitor TG-1701

BTK is a member of the Tec family of non-receptor tyrosine kinases and is a key signaling enzyme expressed in all hematopoietic cells types except T lymphocytes and natural killer (NK) cells. BTK is a key component of the B-cell receptor signaling pathways that regulate B-cell differentiation, activation, proliferation, and survival (Kurosaki, T., Curr Op Imm 12:276-281 (2000); Schaeffer, E. M. and Schwartzberg, P. L., Curr Op Imm 12: 282-288 (2000)). In addition, BTK plays a role in a number of other hematopoetic cell signaling pathways, e.g., Toll like receptor (TLR) and cytokine receptor-mediated TNF-alpha production in macrophages, IgE receptor (FcepsilonRI) signaling in mast cells, inhibition of Fas/APO-1 apoptotic signaling in B-lineage lymphoid cells, and collagen-stimulated platelet aggregation. See, e.g., Jeffries, C. A. et al., J. Biol. Chem. 278:26258-26264 (2003); Horwood, N. J. et al., The Journal of Experimental Medicine 197:1603-1611 (2003); Iwaki, S. et al., J. Biol. Chem. 280:40261-40270 (2005). BTK functions as an important regulator of cell proliferation and cell survival in various B-cell malignancies. Several BTK inhibitors, such as, e.g., ibrutinib (IMBRUVICA®) and acalabrutinib (CALQUENCE®), have been FDA-approved for the treatment of e.g., CLL and MCL. Zanubrutinib (BRUKINSA®) has been FDA-approved for the treatment of MCL and more recently, WM and MZL.

The BTK inhibitor used in the methods and kits described herein is (R)-4-amino-1-(1-(but-2-ynoyl)pyrrolidin-3-yl)-3-(4-(2,6-difluorophenoxy)phenyl)-1,6-dihydro-7H-pyrrolo[2,3-d]pyridazin-7-one (TG-1701), or an isomer, polymorph, enantiomer, pharmaceutically acceptable salt, solvate, or prodrug thereof. The alternate chemical name for TG-1701 is R)-4-amino-1-(1-(but-2-ynoyl)pyrrolidin-3-y 1)-3-(4-(2,6-difluorophenoxy) phenyl)-1H-pyrrolo[2,3-d]pyridazin-7 (6H)-one.

In a certain aspects, the BTK inhibitor is (R)-4-amino-1-(1-(but-2-ynoyl)pyrrolidin-3-yl)-3-(4-(2,6-difluorophenoxy)phenyl)-1,6-dihydro-7H-pyrrolo[2,3-d]pyridazin-7-one, also known as TG-1701, SHR-1459, or EBI-1459. The terms “TG-1701” or “BTK inhibitor TG-1701” are used interchangeably and will be used predominantly herein.

The chemical formula of TG-1701 is C₂₆H₂₁F₂N₅O₃, and its molecular weight is 489.48 g/mol. TG-1701 has the following chemical structure:

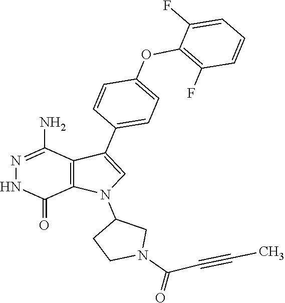 [SMILES: CC#CC(=O)N1CCC(n2cc(-c3ccc(Oc4c(F)cccc4F)cc3)c3c(N)n[nH]c(=O)c32)C1]

TG-1701 is described in PCT Publication No. WO2016/007185 and U.S. Pat. Nos. 9,951,077 and 10,323,037, which are all incorporated by reference in their entireties. Crystalline forms of TG-1701 are described in PCT Publication No. WO2017/118277 and U.S. Pat. No. 10,626,116, and are also incorporated by reference in their entireties.

TG-1701 is an orally available, covalently-bound, irreversible, selective inhibitor of BTK. TG-1701 has been shown to exhibit superior selectivity for BTK compared to other clinically-available BTK inhibitors, such as ibrutinib and acalabrutinib. See, e.g., Normant, E. et al., Abstract 3973, European Hematology Association Annual Meeting, Stockholm, Sweden (Jun. 14, 2018). TG-1701 was evaluated and compared to ibrutinib and/or acalabrutinib in numerous enzyme based, cell-based, and animal models. For example, TG-1701 was shown to be as active as ibrutinib (having comparable kinase inhibition IC₅₀s against BTK—3 nM and 1.5 nM, respectively), but with improved selectivity for BTK in an in vitro whole kinome screening. Id. In addition, TG-1701 was 90-fold less active on EGFR compared to BTK with an IC₅₀ of 270 nM and 3 nM respectively. Ibrutinib, however, was only 4.3-fold less active on EGFR compared to BTK with an IC₅₀ of 6.4 nM and 1.5 nM respectively. Id.

The inhibitory effect of TG-1701 on cell proliferation was measured in several cell lines (B-cell lymphomas). TG-1701 inhibited the growth of the follicular lymphoma (FL) DOHH-2, mantle cell lymphoma (MCL) Mino, and DLBCL SU-DHL-6 cell lines with IC₅₀s of 369, 449, and 313 nM, respectively. TG-1701 inhibited IgM-activated BCR pathway in DOHH-2 cells, in particular, the phosphorylation of BTK, PLCy2, and ERK1/2. In a cell-based assay, TG-1701 blocked IgM-dependent CD69 expression, adhesion of JEKO cells to VCAM-1, and CXCL12-dependent migration. Id.

A fluorescent BTK-occupancy assay was developed and validated in vivo, in the spleen of mice, where BTK was found to be completely occupied after administration of a single dose of TG-1701 at 12.5 mg/kg. In vivo, the anti-tumor efficacy of TG-1701 was assessed in several lymphoma xenograft models, e.g., SU-DHL-6 (GCB-DLBCL), Mino (MCL), and OCI-Ly10 (ABC-DLBCL), where TG-1701 showed potent anti-tumor activity equivalent to or greater than ibrutinib and similar to the recently approved BTK inhibitor, acalabrutinib. In addition, the pharmacokinetic profile of TG-1701 allows for a once a day dosing. TG-1701 is a novel and highly-selective, irreversible BTK inhibitor with potent in vitro and in vivo activity. Id.

Further, in dose-escalation studies, TG-1701 has shown an improved safety profile and lower toxicity compared to ibrutinib and certain other BTK inhibitors. Notably, in a comparison of Grade 3 or greater adverse events (AEs) between ibrutinib, acalabrutinib, and TG-1701, the % incidence of atrial fibrillation (Afib) was 3.4% (n=263), 4.5% (n=266), and 1.9% (N=106), respectively. Thus, the % incidence of grade 3 or higher Afib in patients taking TG-1701 was almost half less than the incidence when taking ibrutinib or acalabrutinib. Following a similar trend, when evaluating the same number of patients for Afib of any grade in patients talking ibrutinib, acalabrutinib, and TG-1701, the % incidence of any grade Afib was 15.6% (n=263), 9% (n=266), and 3.8% (N=106), respectively. Thus, the % incidence of any grade Afib in patients taking TG-1701 was more than half less than the incidence when taking ibrutinib or acalabrutinib.

In addition, unlike previous generations of BTK inhibitors, TG-1701 does not impair FcγR-driven ADCC and ADCP (antibody-dependent cellular phagocytosis) activity of anti-CD20 antibodies (e.g., ublituximab), but instead, cooperates with ublituximab in in vitro and in vivo models of BTKi-sensitive and BTKi-resistant B-NHL. See, Ribeiro, M. L. et al., Cancer Res. 80 (16 Supplement): Abstract 2205 (2020). Across cell lines, the ADCC activity of ublituximab was not significantly different in the presence of TG-1701 compared to the monotherapy ADCC activity of ublituximab. In contrast to TG-1701, the ADCC activity of ublituximab was attenuated in combination with ibrutinib. A similar trend was shown for ADCP activity. The ADCP activity of rituximab was diminished in the presence of ibrutinib, while conserved to a greater extent in combination with TG-1701. For ublituximab, increasing concentrations of ibrutinib resulted in corresponding decreases in ADCP activity; however, in combination with TG-1701, ublituximab's ADCP was conserved at all dose levels tested. Id. Thus, unlike previous generations of BTK inhibitors, TG-1701 conserves the ADCC and ADCP activity of anti-CD20 antibodies, including ublituximab.

### B-Cell Malignancies

In some aspects, the B-cell malignancy to be treated is in a human subject.

In some aspects, the B-cell malignancy is selected from the group consisting of acute lymphocytic leukemia (ALL), acute myeloid leukemia (AML), chronic lymphocytic leukemia (CLL), small lymphocytic lymphoma (SLL), multiple myeloma (MM), non-Hodgkin's lymphoma (NHL), mantle cell lymphoma (MCL), follicular lymphoma (FL), Waldenstrom's macroglobulinemia (WM), diffuse large B-cell lymphoma (DLBCL), marginal zone lymphoma (MZL), including extranodal and nodal MZL, hairy cell leukemia (HCL), Burkitt's lymphoma (BL), and Richter's transformation.

In some aspects, the B-cell malignancy is selected from the group consisting of chronic lymphocytic leukemia (CLL), small lymphocytic lymphoma (SLL), non-Hodgkin's lymphoma (NHL), mantle cell lymphoma (MCL), follicular lymphoma (FL), diffuse large B-cell lymphoma (DLBCL), and marginal zone lymphoma (MZL).

In some aspects, the B-cell malignancy is CLL.

In some aspects, the B-cell malignancy overexpresses CD20.

In some aspects, the B-cell malignancy is refractory to chemotherapy.

In some aspects, the B-cell malignancy is refractory to non-TGR-1202 PI3K-delta inhibitors.

In some aspects, the B-cell malignancy is refractory to non-ublituximab anti-CD20 antibodies.

In some aspects, the B-cell malignancy is refractory to any agent described herein, i.e., an anti-CD20 antibody, a PI3K delta selective inhibitor, or a BTK inhibitor, when said agent was administered individually to a subject (i.e., used as a monotherapy).

In some aspects, the B-cell malignancy is refractory to a non-TG-1701 BTK inhibitor. In some aspects, the B-cell malignancy is refractory to ibrutinib.

In some aspects, the B-cell malignancy is refractory to rituximab.

In some aspects, the B-cell malignancy has relapsed.

In some aspects, the human subject has one or more genetic mutations selected from the group consisting of 17p del, 11q del, p53, unmutated IgVH together with ZAP-70+ and/or CD38+, trisomy 12, and complex karyotype.

### Administration of the Combination

In some aspects, the agents (i.e., i, ii, and iii, as described herein) to be used in combination in the methods described herein, are administered to a subject separately.

In some aspects, the agents (i.e., i, ii, and iii) to be used in combination in the methods described herein, are administered to a subject sequentially, although, as noted below, the particular order of administration is not an issue. In some aspects, the agents (i.e., i and iii), which will be used in combination with the anti-CD20 antibody (i.e., ii) in the methods described herein, are administered to the subject simultaneously or sequentially. In some aspects, the agents (i.e., i and iii) are contained in the same pharmaceutical composition. In some aspects, the agents (i.e., i and iii) are formulated for oral administration.

In some aspects, the combination of agents is sequentially administered in induction, consolidation, and/or maintenance regimens.

In some aspects, two of the agents i, ii, or iii, are administered together in order to induce a partial anti-tumor response, followed by administration of the third agent to enhance the anti-tumor response. In some aspects, a complete anti-tumor response (CR) is observed following administration of all agents (e.g., i, ii, and iii, as disclosed herein) to said subject. In some aspects, a subject administered any of the methods described herein achieves a complete response with minimal residual disease (MRD).

In some aspects, a subject administered any of the methods described herein achieves a partial response (PR) when all three agents are administered in combination. In some aspects, a subject administered any of the methods described herein achieves a partial response (PR) or a complete response (CR) that is durable for at least about 24 weeks to about 36 months.

In some aspects, at least one of the agents, i, ii, and/or iii, is administered in a maintenance therapy in order to keep the B-cell malignancy from returning after successful treatment. In some aspects, the agent is administered in maintenance therapy for an extended period of time, e.g., until unmanageable toxicity, or disease progression occurs. In some aspects, the maintenance therapy ends when disease progression occurs. In some aspects, ublituximab infusion continues being administered every three months until disease progression. In some aspects, umbralisib single agent therapy is administered daily until disease progression. In some aspects, TG-1701 single agent therapy is administered daily until disease progression. In some aspects, umbralisib and TG-1701 therapies are administered daily until disease progression.

In some aspects, the methods described herein further comprises administering to the subject at least one additional therapeutic agent for treating a B-cell malignancy. In some aspects, the at least one additional therapeutic agent is selected from the group consisting of mitotic inhibitors, alkylating agents, anti-metabolites, anthracyclines, vinca alkaloids, plant alkaloids, nitrogen mustards, proteasome inhibitors, intercalating antibiotics, growth factor inhibitors, cell-cycle inhibitors, biological response modifiers, anti-hormones, angiogenesis inhibitors, anti-androgens, DNA interactive agents, purine analogues, topoisomerase I inhibitors, topoisomerase II inhibitors, tubulin interacting agents, hormonal agents, thymidilate synthase inhibitors, non-BTK and non-PI3K-delta tyrosine kinase inhibitors, angiogenesis inhibitors, EGF inhibitors, VEGF inhibitors, CDK inhibitors, SRC inhibitors, c-Kit inhibitors, Her1/2 inhibitors, BET bromodomain inhibitors, inhibitors of myc, anti-tumor antibodies, monoclonal antibodies directed against growth factor receptors, monoclonal antibodies directed against checkpoint inhibitors, monoclonal antibodies against CD19 and/or CD47, protein kinase modulators, radioactive isotopes, immunotherapies, glucocorticoids, and any combinations thereof.

In some aspects, the at least one additional therapeutic agent is selected from the group consisting of a proteasome inhibitor, Bortezomib (Velcade®), Carfilzomib (PR-171), PR-047, disulfiram, lactacystin, PS-519, eponemycin, epoxomycin, aclacinomycin, CEP-1612, MG-132, CVT-63417, PS-341, vinyl sulfone tripeptide inhibitors, ritonavir, PI-083, (+/−)-7-methylomuralide, (−)-7-methylomuralide, lenalidomide, bendamustine, TG-1501, TG-1601, TG-1801, and any combinations thereof.

In some aspects, the at least one additional therapeutic agent is an anti-cancer agent selected from the group consisting of DNA interactive agents, such as cisplatin or doxorubicin; topoisomerase II inhibitors, such as etoposide; topoisomerase I inhibitors such as CPT-11 or topotecan; tubulin interacting agents, such as paclitaxel, docetaxel or the epothilones (for example ixabepilone), either naturally occurring or synthetic; hormonal agents, such as tamoxifen; thymidilate synthase inhibitors, such as 5-fluorouracil; and anti-metabolites, such as methotrexate; other tyrosine kinase inhibitors such as Iressa and OSI-774; angiogenesis inhibitors; EGF inhibitors; VEGF inhibitors; CDK inhibitors; SRC inhibitors; c-Kit inhibitors; Her1/2 inhibitors and monoclonal antibodies directed against growth factor receptors such as erbitux (EGF) and herceptin (Her2); and other protein kinase modulators. Other anti-cancer agents that could be used in the methods and kits of the invention will be known to those skilled in the oncology art.

In some aspects, the at least one additional therapeutic agent is a combination of chemotherapies, known to treat hematological malignancies, such as, e.g., “CHOP” (a combination including (i) cyclophosphamide such as cytoxan, (ii) doxorubicin or other topoisomerase II inhibitors such as adriamycin, (iii) vincristine or other vincas such as oncovin; and (iv) a steroid such as hydrocortisone or prednisolone); “R-CHOP” (a combination including rituxan, cyclophosphamide, doxorubicin, vincristine, and prednisone); “ICE” (a combination including ifosfamide, carboplatin, and etoposide); “R-ICE” (a combination including rituxan, ifosfamide, carboplatin, and etoposide); “R-ACVBP” (a combination of rituximab, doxorubicin, cyclophosphamide, vindesine, bleomycin and prednisone); “DA-EPOCH-R” (a combination of dose-adjusted etoposide, doxorubicin, cyclophosphamide, vincristine, prednisone and rituximab); “R-bendamustine” (a combination of bendamustine and rituximab); “GemOx or R-GemOx” (a combination of gemcitabine and oxaliplatin, with or without rituximab); and “DHAP” (a combination including dexamethasone, cytarabine, and cisplatin).

Any oncolytic agent that is routinely used in a cancer therapy context finds use in the therapeutic methods of the present disclosure. For example, the U.S. Food and Drug Administration (FDA) maintains a formulary of oncolytic agents approved for use in the United States. International counterpart agencies to the FDA maintain similar formularies. Those skilled in the art will appreciate that the “product labels” required on all U.S. approved chemotherapeutics describe approved indications, dosing information, toxicity data, and the like, for the exemplary agents.

The combination of agents comprising a PI3K-delta inhibitor, an anti-CD20 antibody, and a BTK inhibitor (or more than one of any or all agents) can be administered in any order or at any interval as determined by those skilled in the art. For example, a PI3K-delta inhibitor of formula A, ublituximab or an anti-CD20 antibody that binds to the same epitope as ublituximab, and a BTK inhibitor can be administered sequentially (in any order), simultaneously, or via any combination of sequential and simultaneous administrations. Any combination of a PI3K-delta inhibitor of formula A, ublituximab or an anti-CD20 antibody that binds to the same epitope as ublituximab, and a BTK inhibitor can be administered in the same pharmaceutical compositions or in separate pharmaceutical compositions. For example, a PI3K-delta inhibitor of formula A and a BTK inhibitor can be administered in the same pharmaceutical composition.

Administration of the combination of agents, whether simultaneous, sequential (in any order) or both, can be performed according to any number of desired intervals of minutes (e.g., 0-60 minutes), hours (e.g., 0-24 hours), days (e.g., 0-7 days), and/or weeks (e.g., 0-52 weeks), as can be decided and determined by one of skill in the art. The dosing can also vary over time, for example, starting with a once weekly dose for a period of time (e.g., for 1, 2, 3, 4, 5, or 6 weeks) followed by dosing once every two weeks, once every three weeks, once every four weeks, once every five weeks, or once every six weeks.

The PI3K-delta selective inhibitor, the anti-CD20 antibody, and the BTK inhibitor that are to be used in combination in the methods of the invention can be formulated into pharmaceutical compositions for administration to mammals, including humans. The pharmaceutical compositions comprise pharmaceutically acceptable carriers, including, e.g., ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The agents to be used in combination in accordance with the methods of the invention can be administered by any suitable method, e.g., orally, parenterally, intraventricularly, by inhalation spray, topically, rectally, nasally, buccally, vaginally or via an implanted reservoir. The mode of administration for each agent does not have to be the same.

In some aspects, the PI3K-delta inhibitor (e.g., TGR-1202) is administered orally.

In some aspects, BTK inhibitor TG-1701 is administered orally.

As used herein, the term “parenteral” includes subcutaneous, intravenous, intramuscular, intra-articular, intra-synovial, intrasternal, intrathecal, intrahepatic, intralesional and intracranial injection or infusion techniques. Parenteral formulations can be a single bolus dose, an infusion, or a loading bolus dose followed with a maintenance dose. These compositions can be administered at specific fixed or variable intervals, e.g., once a day, or on an “as needed” basis. In a preferred aspect, the anti-CD20 antibody ublituximab is administered intravenously (IV), preferably by infusion.

Certain pharmaceutical compositions can be orally administered in an acceptable dosage form including, e.g., capsules, tablets, aqueous suspensions, or solutions. Certain pharmaceutical compositions also can be administered by nasal aerosol or inhalation. Such compositions can be prepared as solutions in saline, employing benzyl alcohol or other suitable preservatives, absorption promoters to enhance bioavailability, and/or other conventional solubilizing or dispersing agents.

A specific dosage and treatment regimen for any particular patient will depend upon a variety of factors, including the particular therapeutic agents used, the patient's age, body weight, general health, sex, and diet, and the time of administration, rate of excretion, drug combination, and the severity of the disease being treated. In some cases, dosages may need to be modified based on scan or biopsy results. Judgment of such factors by medical caregivers is within the ordinary skill in the art. Dosage will also depend on the individual patient to be treated, the route of administration, the type of formulation, the characteristics of the compound used, tumor burden, and the desired effect. The amount used can be determined by pharmacological and pharmacokinetic principles well known in the art.

In some aspects, the anti-CD20 antibody is ublituximab and it is administered at a dose from: about 450 mg to about 1200 mg, about 600 to about 1200 mg, about 600 to about 1000 mg, about 600 to about 900 mg, about 600 mg, about 700 mg, about 800 mg, or about 900 mg. In certain aspects, ublituximab is administered at a dose of about 900 mg. In certain aspects, ublituximab is administered at a dose of 900 mg.

Ublituximab may be administered about twice every week, about once every 1 to 9 weeks, about once every week, about once every 2 weeks, about once every 3 weeks, about once every 4 weeks, about once every 5 weeks, about once every 6 weeks, about once every 7 weeks, about once every 8 week, or about once every 9 weeks. One skilled in the art will appreciate that the dosage of ublituximab and/or frequency of administering ublituximab may change during the course of therapy (lowered or increased) depending upon the patient's clinical response, side effects, etc.

In some aspects, ublituximab is administered on day 1, 8, and 15 of cycle 1 and cycle 2 and day 1 on cycles 4, 6, 9, and 12, wherein each cycle is about 28 days. In some aspects, each cycle is 28 days.

In some aspects, a PI3K-delta selective inhibitor of formula A is administered once a day at a dosage from: about 200 mg to about 1200 mg, about 400 mg to about 1000 mg, about 400 mg to about 800 mg, about 400 mg, about 500 mg, about 600 mg, about 700 mg, about 800 mg, about 900 mg, about 1000 mg, or about 1200 mg.

In some aspects, the PI3K-delta selective inhibitor of formula A is (S)-2-(1-(4-amino-3-(3-fluoro-4-isopropoxyphenyl)-1H-pyrazolo[3,4-d]pyrimidin-1-yl)ethyl)-6-fluoro-3-(3-fluorophenyl)-4H-chromen-4-one PTSA salt (TGR-1202) and it is administered at a dose from about 400 mg to about 1200 mg per day. In some aspects, the TGR-1202 is administered at a dose of about 400 mg per day. In some aspects, the TGR-1202 is administered at a dose of about 600 mg per day. In some aspects, the TGR-1202 is administered at a dose of about 800 mg per day.

The BTK inhibitor used in the methods and kits disclosed herein is (R)-4-amino-1-(1-(but-2-ynoyl)pyrrolidin-3-yl)-3-(4-(2,6-difluorophenoxy)phenyl)-1,6-dihydro-7H-pyrrolo[2,3-d]pyridazin-7-one, (also known as TG-1701, SHR-1459, or EBI-1459), or an isomer, polymorph, enantiomer, pharmaceutically acceptable salt, solvate, or prodrug thereof. The alternate chemical name for TG-1701 is R)-4-amino-1-(1-(but-2-ynoyl)pyrrolidin-3-y 1)-3-(4-(2,6-difluorophenoxy) phenyl)-1H-pyrrolo[2,3-d]pyridazin-7 (6H)-one. The terms “TG-1701” or “BTK inhibitor TG-1701” are used interchangeably and will be used predominantly herein.

In a certain aspects, the BTK inhibitor is (R)-4-amino-1-(1-(but-2-ynoyl)pyrrolidin-3-yl)-3-(4-(2,6-difluorophenoxy)phenyl)-1,6-dihydro-7H-pyrrolo[2,3-d]pyridazin-7-one.

In some aspects, the TG-1701 is administered once daily at a dosage from: about 100 mg to about 400 mg. In certain aspects, TG-1701 is administered at a dose of about 100 mg per day. In certain aspects, TG-1701 is administered at a dose of about 200 mg per day. In certain aspects, TG-1701 is administered at a dose of about 300 mg per day. In certain aspects, TG-1701 is administered at a dose of about 400 mg per day. In some aspects, the TG-1701 is administered once a day (QD). TG-1701 is administered orally.

Further, in dose-escalation studies of TG-1701 in combination with U2, as described herein, lower levels of dosages of umbralisib may be administered, while still maintaining anti-tumor activity. For example, the ability to utilize lower dosages of umbralisib, while maintaining anti-tumor activity when combined with TG-1701 has been demonstrated. The lower dose of umbralisib may be 50%, 55%, 60%, 65%, 70%, 75%, 80%, 85%, or 90% of the starting dose administered. The use of lower dosages while not sacrificing efficacy is advantageous in order to mitigate or reduce adverse effects while receiving cancer therapy.

Supplementary active compounds can also be incorporated into the methods and kits of the present invention. For example, an anti-CD20 antibody, a PI3K-delta selective inhibitor, and BTK inhibitor 1701 can be coformulated with and/or coadministered with one or more additional therapeutic agents. As non-limiting examples, the methods and kits could be coformulated with anti-cancer agents such as DNA interactive agents, such as cisplatin or doxorubicin; topoisomerase II inhibitors, such as etoposide; topoisomerase I inhibitors such as CPT-11 or topotecan; tubulin interacting agents, such as paclitaxel, docetaxel or the epothilones (for example ixabepilone), either naturally occurring or synthetic; hormonal agents, such as tamoxifen; thymidilate synthase inhibitors, such as 5-fluorouracil; and anti-metabolites, such as methotrexate; other tyrosine kinase inhibitors such as Iressa and OSI-774; angiogenesis inhibitors; EGF inhibitors; VEGF inhibitors; CDK inhibitors; SRC inhibitors; c-Kit inhibitors; Her1/2 inhibitors and monoclonal antibodies directed against growth factor receptors such as erbitux (EGF) and herceptin (Her2); and other protein kinase modulators. The additional active agent can also be a proteasome inhibitor, Bortezomib (Velcade®), Carfilzomib (PR-171), PR-047, disulfiram, lactacystin, PS-519, eponemycin, epoxomycin, aclacinomycin, CEP-1612, MG-132, CVT-63417, PS-341, vinyl sulfone tripeptide inhibitors, ritonavir, PI-083, (+/−)-7-methylomuralide, (−)-7-methylomuralide, lenalidomide (Revlimid®), or a combination thereof.

### Kits

In one aspect, provided herein is a kit comprising a PI3K-delta selective inhibitor of formula A, an anti-CD20 antibody that is ublituximab or an antibody that binds to the same epitope as ublituximab, and BTK inhibitor TG-1701. In some aspects, other agents that can be used to perform the methods described herein, and combinations thereof, are included in the kit. Such kits can include, for example, other compounds and/or compositions to treat B-cell malignancies known to those skilled in the art, a device(s) for administering the compounds and/or compositions, and written instructions in a form prescribed by a governmental agency regulating the manufacture, use or sale of pharmaceuticals or biological products.

In some aspects, a kit comprises (a) a PI3K-delta selective inhibitor of formula A (e.g., TGR-1202), or a stereoisomer thereof, or a pharmaceutically acceptable salt, solvate, or prodrug thereof; ublituximab or an anti-CD20 antibody or fragment thereof that binds to the same epitope as ublituximab; and BTK inhibitor TG-1701, and (b) instructions for using said PI3K-delta selective inhibitor in combination with ublituximab or an anti-CD20 antibody or fragment thereof that binds to the same epitope as ublituximab and TG-1701.

In some aspects, the PI3K-delta selective inhibitor in the kit is (S)-2-(1-(4-amino-3-(3-fluoro-4-isopropoxyphenyl)-1H-pyrazolo[3,4-d]pyrimidin-1-yl)ethyl)-6-fluoro-3-(3-fluorophenyl)-4H-chromen-4-one (umbralisib).

In some aspects, the PI3K-delta selective inhibitor in the kit is (S)-2-(1-(4-amino-3-(3-fluoro-4-isopropoxyphenyl)-1H-pyrazolo[3,4-d]pyrimidin-1-yl)ethyl)-6-fluoro-3-(3-fluorophenyl)-4H-chromen-4-one PTSA salt (umbralisib PTSA salt).

In some aspects, the kit further comprises ublituximab or an anti-CD20 antibody or fragment thereof that binds to the same epitope as ublituximab.

In some aspects, the kit further comprises ublituximab.

One skilled in the art will readily recognize that the disclosed combination of agents (antibodies and small molecule inhibitors) described herein for use in the methods of the invention can be readily incorporated into one of the established kit formats that are well known in the art.

Further provided are kits comprising (a) a PI3K-delta selective inhibitor of formula A, an anti-CD20 antibody, and BTK inhibitor TG-1701, or a combination thereof; and (b) an additional therapeutic agent. In some aspects, the kit comprises umbralisib, ublituximab, BTK inhibitor TG-1701, and a chemotherapeutic agent selected from the group consisting of DNA interactive agents, such as cisplatin or doxorubicin; topoisomerase II inhibitors, such as etoposide; topoisomerase I inhibitors such as CPT-11 or topotecan; tubulin interacting agents, such as paclitaxel, docetaxel or the epothilones (for example ixabepilone), either naturally occurring or synthetic; hormonal agents, such as tamoxifen; thymidilate synthase inhibitors, such as 5-fluorouracil; and anti-metabolites, such as methotrexate; other tyrosine kinase inhibitors such as Iressa and OSI-774; angiogenesis inhibitors; EGF inhibitors; VEGF inhibitors; CDK inhibitors; SRC inhibitors; c-Kit inhibitors; Her1/2 inhibitors and monoclonal antibodies directed against growth factor receptors such as erbitux (EGF) and herceptin (Her2); and other protein kinase modulators.

In certain aspects, the kit comprises umbralisib, ublituximab, BTK inhibitor TG-1701, and an additional therapeutic agent selected from the group consisting of mitotic inhibitors, alkylating agents, anti-metabolites, anthracyclines, vinca alkaloids, plant alkaloids, nitrogen mustards, proteasome inhibitors, intercalating antibiotics, growth factor inhibitors, cell-cycle inhibitors, biological response modifiers, anti-hormones, angiogenesis inhibitors, anti-androgens, DNA interactive agents, purine analogues, topoisomerase I inhibitors, topoisomerase II inhibitors, tubulin interacting agents, hormonal agents, thymidilate synthase inhibitors, non-BTK and non-PI3K-delta tyrosine kinase inhibitors, angiogenesis inhibitors, EGF inhibitors, VEGF inhibitors, CDK inhibitors, SRC inhibitors, c-Kit inhibitors, Her1/2 inhibitors, BET bromodomain inhibitors, inhibitors of myc, anti-tumor antibodies, monoclonal antibodies directed against growth factor receptors, monoclonal antibodies directed against checkpoint inhibitors, monoclonal antibodies against CD19 and/or CD47, protein kinase modulators, radioactive isotopes, immunotherapies, glucocorticoids, and any combinations thereof.

In certain aspects, the kit comprises umbralisib, ublituximab, BTK inhibitor TG-1701, and an additional therapeutic agent selected from the group consisting of a proteasome inhibitor, Bortezomib (Velcade®), Carfilzomib (PR-171), PR-047, disulfiram, lactacystin, PS-519, eponemycin, epoxomycin, aclacinomycin, CEP-1612, MG-132, CVT-63417, PS-341, vinyl sulfone tripeptide inhibitors, ritonavir, PI-083, (+/−)-7-methylomuralide, (−)-7-methylomuralide, lenalidomide, bendamustine, TG-1501, TG-1601, TG-1801 and any combinations thereof.

Methods of Using Combinations of a PI3K-Delta Selective Inhibitor of Formula A, Ublituximab or an Anti-CD20 Antibody that Binds the Same Epitope as Ublituximab, and BTK Inhibitor TG-1701

Combinations of a PI3K-delta selective inhibitor of Formula A, ublituximab or an anti-CD20 antibody that binds the same epitope as ublituximab, and BTK inhibitor TG-1701, can be used in methods of treating B-cell malignancies in a subject.

In some aspects, a PI3K-delta selective inhibitor of Formula A can be used in the manufacture of a medicament for the treatment of a B-cell malignancy, wherein the PI3K-delta selective inhibitor of Formula A is to be administered in combination (e.g., sequentially or simultaneously) with an anti-CD20 antibody that is ublituximab or an antibody that binds to the same epitope as ublituximab, and the BTK inhibitor TG-1701. In addition, an anti-CD20 antibody can be used in the manufacture of a medicament for the treatment of a B-cell proliferative disorder, wherein the anti-CD20 antibody is to be administered in combination (e.g., sequentially or simultaneously) with a PI3K-delta selective inhibitor of Formula A and BTK inhibitor TG-1701. In some aspects, the anti-CD20 antibody is ublituximab. In some aspects, the PI3K-delta selective inhibitor is TGR-1202. In some aspects, the anti-CD20 antibody is ublituximab, and the PI3K-delta selective inhibitor is TGR-1202.

The invention further provides a method of inhibiting PI3K-delta isoform and/or CD20, and/or BTK in a subject by administering to the subject an effective amount of the agents of the present invention in combination.

The invention further provides a method of treating, preventing, and/or inhibiting a PI3K-delta-mediated disease, disorder or condition and/or a CD20-mediated disease, disorder, or condition (such as cancer or other proliferative disease or disorder) and/or a BTK-mediated disease, disorder, or condition in a patient by administering to the patient an effective amount of the agents of the present invention in combination.

The invention further provides a method of treating a PI3K-delta isoform- and/or CD20- or BTK-associated disease, disorder or condition in a patient by administering to the patient an effective amount of the agents of the present invention in combination. In some aspects, the amount of the agents administered in combination is sufficient to treat a PI3K-delta isoform- and/or CD20- and/or BTK-associated disease, disorder, or condition by selective inhibition of PI3K-delta and/or CD20 and/or BTK.

In some aspects, the invention further provides a method of treating a B-cell malignancy by administering to a patient in need of such treatment an effective amount of a PI3K delta selective inhibitor, an anti-CD20 antibody, and BTK inhibitor TG-1701. In some aspects, the anti-CD20 antibody is ublituximab. In some aspects, the PI3K-delta selective inhibitor is umbralisib (TGR-1202). In some aspects, the anti-CD20 antibody is ublituximab and the PI3K-delta selective inhibitor is umbralisib. In some aspects, the anti-CD20 antibody is ublituximab, the PI3K-delta selective inhibitor is umbralisib, and the BTK inhibitor is TG-1701.

In some aspects, the invention further provides a method of treating a B-cell malignancy by administering to a patient in need of such treatment an effective amount of a PI3K delta selective inhibitor of formula A, an anti-CD20 antibody that is ublituximab or an antibody that binds to the same epitope as ublituximab, and BTK inhibitor TG-1701, as described herein. In some aspects, the amounts of the agents administered in combination are sufficient to treat the B-cell malignancy by selective inhibition of PI3K-delta, and/or inhibition of CD20, and/or inhibition of BTK. In some aspects, the anti-CD20 antibody is ublituximab. In some aspects, the PI3K-delta selective inhibitor is umbralisib. The BTK inhibitor is TG-1701. In some aspects, the anti-CD20 antibody is ublituximab and the PI3K-delta selective inhibitor is TGR-1202. In some aspects, the anti-CD20 antibody is ublituximab, the PI3K-delta selective inhibitor is umbralisib, and the BTK inhibitor is TG-1701.

The invention further provides a method for treating a B-cell malignancy by administering to a patient in need of such treatment an effective amount of a combination of the agents of the present invention, in further combination (simultaneously or sequentially) with at least one other anti-cancer agent. In one embodiment, the amount of the PI3K-delta selective inhibitor of Formula A administered is sufficient to treat (or facilitate treatment of) the B-cell malignancy by selective inhibition of PI3K-delta.

The combinations of the agents of the present invention are particularly useful in the treatment of a variety of B-cell cancers, such as, but not limited to, e.g., acute lymphocytic leukemia (ALL), acute myeloid leukemia (AML), chronic lymphocytic leukemia (CLL), small lymphocytic lymphoma (SLL), multiple myeloma (MM), non-Hodgkin's lymphoma (NHL), mantle cell lymphoma (MCL), follicular lymphoma (FL), Waldenstrom's macroglobulinemia (WM), diffuse large B-cell lymphoma (DLBCL), marginal zone lymphoma (MZL), hairy cell leukemia (HCL), Burkitt's lymphoma (BL), and Richter's transformation.

Those skilled in the art would appreciate that other types of lymphomas or leukemias would find use in the combination of agents of the invention, such as, e.g., B-cell prolymphocytic leukemia, lymphoplasmacytic lymphoma, plasma cell myeloma/plasmacytoma, Hodgkin's lymphoma, and Burkitt's lymphoma/Burkitt's cell leukemia.

In some aspects, Non-Hodgkin's Lymphoma (NHL) is aggressive NHL or indolent NHL. Examples of aggressive NHL include diffuse large B-cell lymphoma (DLBCL) either de novo or arising from a previously indolent NHL, T/NK cell neoplasms, anaplastic large cell lymphoma, precursor B-lymphoblastic leukemia/lymphoma, Burkitt's lymphoma, primary CNS lymphoma, mantle cell lymphoma (MCL), polymorphic post-transplantation lymphoproliferative disorder (PTLD), AIDS-related lymphoma, true histiocytic lymphoma, Richter's transformation, and blastic NK-cell lymphoma. The most common type of aggressive NHL is diffuse large cell lymphoma. Non-limiting examples of indolent NHL include follicular lymphoma, small lymphocytic lymphoma, marginal zone lymphoma (such as extranodal marginal zone lymphoma (also called mucosa associated lymphoid tissue—MALT lymphoma), nodal marginal zone B-cell lymphoma (monocytoid B-cell lymphoma), splenic marginal zone lymphoma), and lymphoplasmacytic lymphoma (Waldenstrom's macroglobulinemia). In some aspects, aggressive NHL and indolent NHL are alternately referred to as high-grade NHL and low-grade NHL, respectively. In some aspects, a subject has aggressive NHL or indolent NHL. In some aspects, a subject has a combination of both aggressive and indolent NHL simultaneously.

In some aspects, a patient has a condition selected from the group consisting of mantle cell lymphoma (MCL), diffuse large B cell lymphoma (DLBCL), follicular lymphoma (FL), acute lymphocytic leukemia (ALL), acute myeloid leukemia (AML), chronic lymphocytic leukemia (CLL), small lymphocytic lymphoma (SLL), multiple myeloma (MM), and marginal zone lymphoma.

In some aspects, the patient has CLL.

The combination of agents of the present invention as modulators of apoptosis are useful in the treatment, prevention, and inhibition of cancer (including, but not limited to, the types of B-cell malignancies mentioned above).

The combination of agents of the present invention are also useful in the chemoprevention of cancer. Chemoprevention involves inhibiting the development of invasive cancer by blocking the initiating mutagenic event, blocking the progression of pre-malignant cells that have already suffered an insult, or inhibiting tumor relapse. The compounds are also useful in inhibiting tumor angiogenesis and metastasis. One embodiment of the invention is a method of inhibiting tumor angiogenesis or metastasis in a patient by administering an effective amount of one or more compounds of the present invention.

In the aforementioned methods of treatment, one or more additional therapeutic agents can be administered with the combination of agents of the present invention. For example, the combination of agents of the present invention are useful in combining (administered together or sequentially) with known anti-cancer treatments such as radiation therapy or with one or more cytostatic, cytotoxic or anticancer agents, such as, for example, DNA interactive agents, such as cisplatin or doxorubicin; topoisomerase II inhibitors, such as etoposide; topoisomerase I inhibitors such as CPT-11 or topotecan; tubulin interacting agents, such as paclitaxel, docetaxel or the epothilones (for example ixabepilone), either naturally occurring or synthetic; hormonal agents, such as tamoxifen; thymidilate synthase inhibitors, such as 5-fluorouracil; and anti-metabolites, such as methotrexate; other tyrosine kinase inhibitors such as Iressa and OSI-774; angiogenesis inhibitors; EGF inhibitors; VEGF inhibitors; CDK inhibitors; SRC inhibitors; c-Kit inhibitors; Her1/2 inhibitors and monoclonal antibodies directed against growth factor receptors such as erbitux (EGF) and herceptin (Her2); and other protein kinase modulators. The additional active agent can also be a proteasome inhibitor, Bortezomib (Velcade), Carfilzomib (PR-171), PR-047, disulfiram, lactacystin, PS-519, eponemycin, epoxomycin, aclacinomycin, CEP-1612, MG-132, CVT-63417, PS-341, vinyl sulfone tripeptide inhibitors, ritonavir, PI-083, (+/−)-7-methylomuralide, (−)-7-methylomuralide, lenalidomide (Revlimid®), or a combination thereof.

The combination of agents of the present invention are also useful in combining (administered together or sequentially) with one or more steroidal anti-inflammatory drugs (e.g., prednisone or prednisolone), non-steroidal anti-inflammatory drugs (NSAIDs) or immune selective anti-inflammatory derivatives (ImSAIDs).

In some aspects, a patient has a relapsed or refractory (“R/R”) B-cell malignancy. In some aspects, the subject is refractory to chemotherapy treatment, or in relapse after treatment with chemotherapy. In some aspects, the subject is refractory to a non-TGR-1202 PI3K-delta inhibitor. In some aspects, the subject is refractory to an agent (i, ii, or iii) described herein, where the agent was administered individually (i.e., as a monotherapy).

In some aspects, the cancer is resistant to treatment with rituximab. In some aspects, the cancer shows a reduced response to treatment with rituximab. In some aspects, the subject has previously been treated with rituximab.

In a particular embodiment, the methods comprise reducing the level of NF-kappa-B activity, reducing SNAIL expression, increasing RKIP activity, increasing PTEN activity, increasing tumor sensitivity to TRAIL-apoptosis, reducing the level of PI3K-delta activity or a combination thereof in a subject.

In a particular embodiment, the combination of the PI3K-delta inhibitor of formula A, the anti-CD20 antibody ublituximab, and the BTK inhibitor, as described herein, depletes B-cells from human whole blood. In some aspects, the described triplet combination depletes B-cells from human whole blood to a greater extent than either the PI3K-delta inhibitor of formula A, the anti-CD20 antibody ublituximab, or the BTK inhibitor alone depletes B-cells from human whole blood. In some aspects, the combination of the PI3K-delta inhibitor of formula A, the anti-CD20 antibody ublituximab, and the BTK inhibitor depletes B-cells from human whole blood to a greater extent than the sum of the depletion by the PI3K-delta inhibitor of formula A, the depletion by the anti-CD20 antibody ublituximab, and the depletion by the BTK inhibitor.

In some aspects, a PI3K-delta inhibitor of formula A, the anti-CD20 antibody, and the BTK inhibitor are used in a method of treating a disease or disorder associated with excessive B-cell proliferation, wherein the method comprises administration of a PI3K-delta inhibitor of formula A, the anti-CD20 antibody ublituximab, and the BTK inhibitor TG-1701 to a subject in need thereof. In some aspects, a PI3K-delta inhibitor of formula A, the anti-CD20 antibody, and the BTK inhibitor TG-1701 are used in a method of treating a disease or disorder associated with excessive B-cell activity, wherein the method comprises administration of a PI3K-delta inhibitor of formula A, the anti-CD20 antibody, and the BTK inhibitor TG-1701 to a subject in need thereof. In some aspects, a PI3K-delta inhibitor of formula A, the anti-CD20 antibody, and the BTK inhibitor are used in a method of treating a disease or disorder associated with excessive number of B-cells, wherein the method comprises administration of a PI3K-delta inhibitor of formula A, the anti-CD20 antibody, and the BTK inhibitor to a subject in need thereof. In some aspects, the anti-CD20 antibody is ublituximab. In some aspects, the PI3K-delta selective inhibitor is umbralisib. In some aspects, the BTK inhibitor is TG-1701. In some aspects, the anti-CD20 antibody is ublituximab and the PI3K-delta selective inhibitor is umbralisib. In some aspects, the anti-CD20 antibody is ublituximab, the PI3K-delta selective inhibitor is TGR-1202, and the BTK inhibitor is TG-1701.

When the agents of the present disclosure are administered to a subject (e.g., a human subject), the agents can be administered as a composition that comprises a pharmaceutically acceptable carrier or excipient, by any appropriate route, such as intradermal, intramuscular, intraperitoneal, parenteral, intravenous, subcutaneous, intranasal, epidural, oral, sublingual, buccal, intracerebral, intravaginal, transdermal, transmucosal, rectal, by inhalation, or topical. Delivery can be either local or systemic. Pharmaceutical compositions can take the form of solutions, suspensions, emulsions, tablets, pills, pellets, powders, multi-particulates, capsules, capsules containing liquids, capsules containing powders, capsules containing multi-particulates, lozenges, sustained-release formulations, suppositories, transmucosal films, sub-lingual tablets or tabs, aerosols, sprays, or any other form suitable for use. In one embodiment, the composition is in the form of a tablet. In another embodiment, the composition is in the form of a capsule. Other examples of suitable pharmaceutical excipients are described in Remington's Pharmaceutical Sciences 1447-1676 (Alfonso R. Gennaro ed., 19th ed. 1995), incorporated herein by reference in its entirety.

In certain aspects, the agents of the present disclosure are formulated for oral administration in the form of tablets, capsules, gel caps, caplets, lozenges, aqueous or oily solutions, suspensions, granules, powders, emulsions, syrups, or elixirs, for example. The tablets can be compressed, enteric-coated, sugar-coated, film-coated, multiply compressed or multiply layered.

In certain aspects, the agents of the present disclosure are formulated into a pharmaceutical composition for intravenous administration. Typically, such compositions comprise sterile isotonic aqueous buffer. Where necessary, the compositions can also include a solubilizing agent. Generally, the ingredients are supplied either separately or mixed together in unit dosage form, for example, as a dry lyophilized powder or water free concentrate in a hermetically sealed container such as an ampule or sachet indicating the quantity of active agent. Where administered by infusion, the compositions can be dispensed, for example, with an infusion bottle containing sterile pharmaceutical grade water or saline. Where administered by injection, an ampule of sterile water for injection or saline can be provided so that the ingredients can be mixed prior to administration.

### EXAMPLES

### Example 1: Clinical Activity of TG-1701 as Monotherapy and in Combination with Ublituximab and Umbralisib (“U2”) in Patients with B-Cell Malignancies

### BACKGROUND

BTK inhibition is effective in the treatment of CLL and lymphoma, but requires continuous treatment and complete responses (CR) are rare. BTK-based combination regimens have the potential to increase depth of response and to permit time-limited therapy. TG-1701 is a once-daily (QD), covalently-bound BTK inhibitor with improved selectivity for BTK when compared with ibrutinib in an in vitro whole kinome screening (See, e.g., Normant, E. et al., Abstract 3973, European Hematology Association (EHA) Annual Meeting, Stockholm, Sweden (Jun. 14, 2018)). Of note, the combination of TG-1701 with “U2” (the glycoengineered anti-CD20 monoclonal antibody ublituximab and the PI3Kδ-CK1ε dual inhibitor umbralisib) reduced the tumor growth of ibrutinib sensitive (REC1) and ibrutinib resistant (UPN-1res) mouse models of mantle cell lymphoma (MCL). TG-1701 combined with U2 increased infiltration of macrophages and NK cells promoting thereby immune response in the tumor microenvironment (Ribeiro, M. L. et al., Cancer Res 80 (16 Supplement): 2205-2205 (2020)); see also, Abstract No. 2205, American Association for Cancer Research (AACR)(2020).

In this Example, interim results from an ongoing Phase 1/1b human clinical trial of patients with a range of relapsed/refractory (R/R) or treatment naïve (TN) B-cell malignancies are reported. First, safety was evaluated in escalating doses (100 mg, 200 mg, 300 mg, and 400 mg) of oral TG-1701 once daily (QD) monotherapy continuously administered in 28-day cycles. After the safety profile of TG-1701 monotherapy was characterized, a parallel dose escalation (DE) arm of TG-1701 in combination with umbralisib and ublituximab (“TG-1701+U2”) was initiated. Select dose levels of TG-1701 monotherapy were also expanded to better characterize safety and efficacy in patients with chronic lymphocytic leukemia (CLL), Waldenstrom's macroglobulinemia (WM), and Mantle Cell Lymphoma (MCL). All patients were treated until disease progression, unacceptable toxicity, or investigator/patient decision to withdraw.

### Methods

Study Objectives: The primary objectives of the study were: to characterize the safety profile and determine the recommended Phase 2 dose (RP2D) of TG-1701 as a single agent and in combination with ublituximab and umbralisib (“U2”). Other objectives included evaluating the pharmacokinetics (PK), preliminary antitumor activity, and pharmacodynamics (BTK occupancy) of TG-1701.

Dose Escalation: Treatment consisted of escalating doses of oral TG-1701 QD, continuously administered in 28-day (D) cycles (C). Intra-patient dose escalations were permitted in the TG-1701 monotherapy arm.

Patients in the 1701+U2 combination arm received escalating TG-1701 QD+umbralisib 800 mg oral QD+ublituximab 900 mg IV on days 1, 8, and 15 of cycle 1 (C1), and day 1 (D1) of cycles 2, 3, 4, 5, 6, and day 1 of every 3 cycles thereafter (about 3 months), wherein each cycle is 28 days. All patients were treated until disease progression, unacceptable toxicity, or investigator/patient decision to withdraw study consent.

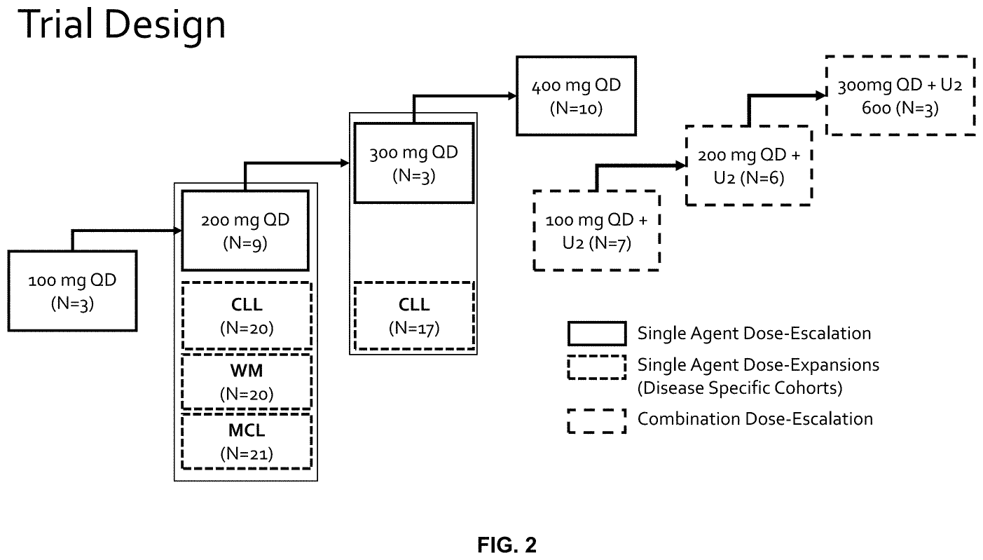

A flow chart of the study schema for the dose-escalation phases for TG-1701 monotherapy, combination TG-1701+Umbralisib+Ublituximab (“U2”), and the disease-specific cohorts CLL, WM, and MCL is depicted in FIG. 1. Trial design for the dose-escalation phases for TG-1701 monotherapy, TG-1701+Umbralisib+Ublituximab (TG-1701+U2) combination therapy, and for single agent dose-expansion in disease-specific cohorts CLL, WM, and MCL, are depicted in the flow chart of FIG. 2.

Key Eligibility Criterion: Patients must have met the following inclusion criteria to be eligible for the study: Relapsed or refractory disease to any prior standard therapy; histologically confirmed B-cell lymphoma or CLL that warrants systemic therapy; B-cell lymphoma subtypes included MCL, WM, SLL, FL, MZL, and DLBCL; for the Disease-Specific Cohorts (CLL, WM, and MCL), previously-untreated patients could be enrolled, if they are considered to be unsuitable for standard front-line hemoimmunotherapy by the treating physician based on the patient's documented comorbidities and risk factors (e.g., 17p deletion or TP53 mutation); adequate organ function, defined as: absolute neutrophil count (ANC)>1,000/μL, platelet count≥50,000/μL, total bilirubin≤1.5 times the upper limit of normal (ULN), ALT/AST≤2.5×ULN if no liver involvement, calculated creatinine clearance>30 mL/min).

Exclusion criteria included prior therapy with a BTK inhibitor, any severe or uncontrolled illness or other conditions that could affect their participation in the study, and concomitant warfarin therapy. (Other anticoagulation therapy was allowed).

### Results

### Baseline Characteristics

Baseline characteristics, e.g., patient demographics and disease characteristics are presented below. Patient demographics from the TG-1701 monotherapy patients and the TG-1701+U2 combination patients in the dose-escalation phase are shown in Table 1.

TABLE 1
Patient Demographics

| | Dose-Escalation Phase | |
|---|---|---|
| | TG-1701 | TG-1701 + U2 |
| Characteristic | (N = 25) | (N = 16) |
| Male sex, N (%) | 14 (56) | 5 (31) |
| Age, years, median (min/max) | 68 (49/86) | 69 (47/79) |
| ≥75 years, N (%) | 7 (28) | 4 (25) |
| ECOG 0/1/2, N(%) | 14(56)/11(44)/0 | 14(87)/2(13)/0 |
| Prior therapies, median (range) | 1 (1-5) | 2 (1-5) |
| Refractory to last prior therapy, N(%) | 7 (28) | 2 (13) |
| Previous anti-CD20 therapy, N(%) | 25 (100) | 16 (100) |
| Treatment-naïve, N (%) | — | — |

Disease characteristics of patients in the three disease-specific cohorts (CLL, WM, and MCL) are shown in Table 2. “ECOG” refers to the Eastern Cooperative Oncology Group (ECOG) scale of performance status. See, Oken, M. M. et al., Am J Clin. Oncol. 5:649-655 (1982).

TABLE 2
Disease Characteristics

| | Disease-specific Cohorts (200 mg QD) | | |
|---|---|---|---|
| | CLL | WM | MCL |
| Characteristic | (N = 20) | (N = 20) | (N = 21) |
| Male sex, N(%) | 7 (35) | 12 (60) | 13 (62) |
| Age, years, median (min/max) | 71(53-87) | 73 (57-92) | 70 (57-85) |
| ≥75 years, N(%) | 4 (20) | 8 (40) | 5 (24) |
| ECOG 0/1/2, N(%) | 7(35)/ | 9(45)/ | 10(48)/ |
| | 13(65)/0 | 10(50)/1(5) | 10(48)/1(4) |
| Prior therapies, median (range) | 1 (0-5) | 1 (0-4) | 3 (0-10) |
| Refractory to last prior therapy, | 2 (10) | 3 (15) | 4 (19) |
| N(%) | | | |
| Previous anti-CD20 therapy, | 14 (93)* | 12 (100)* | 18 (100)* |
| N(%) | | | |
| Treatment-naïve, N(%) | 5 (25) | 8 (40) | 3 (14) |

*Calculation excludes treatment-naïve patients

### Safety

Dose Escalation (N=41 patients):

The median (range) number of cycles of exposure to TG-1701 monotherapy=14 (1-25). Four patients had a dose reduction on TG-1701 monotherapy due to treatment-related adverse events (AEs). Two patients had a dose reduction for TG-1701 (100 mg to 50 mg) due to G3 nausea in the combination arm. Another 3 patients had a dose reduction of umbralisib due to G3-4 ALT elevation. There was one dose limiting toxicity (DLT), G3 ALT elevation at 400 mg. The maximum tolerated dose (MTD) has not been achieved. Dose escalation proceeded until 3 dose levels above full BTK occupancy (100 mg) by TG-1701. No significant changes between pre- and on-treatment diastolic blood pressure nor QTc were noted. No treatment-related death. No treatment discontinuations due to treatment-related adverse events (AEs).

Disease-Specific Cohorts (N=61):

The median number of cycles (range) of TG-1701 monotherapy=7 (1-12). There were no treatment discontinuations due to adverse events.

The incidence of all causality adverse events (AEs) for TG-1701 monotherapy and for TG-1701+U2 combination therapy are shown in Tables 3 and 4, respectively. There have been no Grade 4 AEs in the dose-escalation of TG-1701 monotherapy.

TABLE 3
TG-1701 Monotherapy: All CausalityAEs (Incidence ≥ 10%)

| | Dose escalation | | Disease- | |
|---|---|---|---|---|
| | (100 to 400 mg) | | specific cohorts | |
| Adverse | N = 25 | | (200 mg) N = 61 | |
| event, N (%) | Any Grade | Grade 3 | Any Grade | Grade 3 |
| Constipation | 8 (32) | — | 3 (5) | — |
| Respiratory tract | 7 (28) | 1 (4) | 4 (7) | — |
| infection | | | | |
| Bruising | 7 (28) | — | 5 (8) | — |
| Fatigue | 5 (20) | — | 1 (2) | — |
| Rash | 4 (16) | 1 (4) | 3 (5) | — |
| Nausea | 4 (16) | — | 1 (2) | — |
| Dizziness | 3 (12) | — | 1 (2) | — |
| Headache | 3 (12) | — | 4 (7) | — |
| Diarrhea | 3 (12) | — | 7 (11) | — |
| Epistaxis | 3 (12) | — | 2 (3) | — |
| Hematologic and | | | | |
| lab abnormalities | Any Grade | Grade 3 | Any Grade | Grade ≥ 3 |
| Neutropenia | 6 (24) | 2 (8) | 5 (8) | 3 (5) |
| ALT increased | 6 (24) | 3 (12)^{a} | 2 (3) | 1 (2) |
| AST increased | 5 (20) | 1 (4) | 1 (2) | — |
| Anemia | 4 (16) | — | 4 (7) | 3 (5) |

^{a}All at 400 mg QD. Two cases were brief episodes in asymptomatic pts with normal liver function (total bilirubin within normal range). One case was in the context of significant progression of disease in the liver.

TABLE 4
TG-1701 + U2 Combination Therapy: All Causality AEs (≥15%)

| | Patients (N = 16) | | |
|---|---|---|---|
| Adverse event, N(%) | Any Grade | Grade 3 | Grade 4 |
| Diarrhea | 7 (44) | 1 (6) | — |
| IRR^{a} | 6 (38) | — | — |
| Bruising | 6 (38) | — | — |
| Nausea | 5 (31) | 1 (6) | — |
| Hypertension | 4 (25) | 1 (6) | — |
| Fatigue | 4 (25) | — | — |
| Rash | 3 (19) | — | — |
| Vomiting | 3 (19) | — | — |
| Hematologic and laboratory | | | |
| abnormalities | Any Grade | Grade 3 | Grade 4 |
| Neutropenia | 4 (25) | 1 (6) | 1 (6) |
| ALT increased | 4 (25) | 3 (19)^{b} | 1 (6)^{c} |
| AST increased | 4 (25) | 3 (19) | — |

^{a}IRR: includes the terms “chest tightness,” and “facial flushing.”
^{b}All cases of G3 ALT increased were in patients with normal liver function (total bilirubin within normal range). Two patients continue therapy at a reduced dose of umbralisib (600 mg and 400 mg). The third patient discontinued ublituximab due to serum sickness.
^{c}The G4 ALT increased was symptomatic (vomiting) and with abnormal liver function; the patient has recovered with complete response and remains on study therapy.

### Efficacy

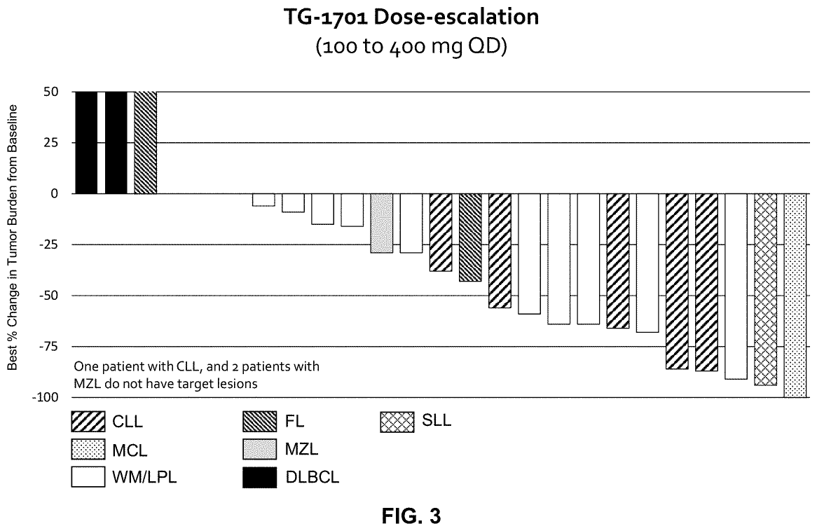

Efficacy results with respect to TG-1701 monotherapy dose-escalation are depicted in Table 5 and FIG. 3.

TABLE 5
Efficacy Dose Escalation (100-400 mg)-TG-1701 Monotherapy

| | Dose-Escalation |
|---|---|
| Response | TG-1701 |
| Category | (N = 23) |
| Median follow-up, mo. (range) | 14 (1-25) |
| Complete response, N (%) | — |
| Very good partial response, N (%) | — |
| Partial response, N (%) | 11 (48) |
| Minor response, N (%) | 1 (4) |
| Stable disease, N (%) | 8 (35) |
| Progressive disease, N (%) | 3 (13) |
| Not evaluable, N (%) | — |
| ORR | 52% |

Efficacy results with respect to the disease-specific cohorts are depicted in Table 6. Evaluable patients had at least one response assessment. Response assessments were made by at least one post baseline scan to assess disease/tumor burden with responses determined according to standard international working group criteria for NHL and CLL, as set forth in Cheson, B. D. et al., J Clin Oncol 25:579-586 (2007) and Hallek, M. et al., Blood 111:5446-5456 (2008), respectively. Response assessments for WM were made according to the sixth international workshop on WM, Owen, R. G. et al., Br J Haematol. 160:171-176(2013).

TABLE 6
Efficacy Disease-Specific Cohorts-TG-1701 monotherapy (200 mg)

| | Disease-Specific Cohorts 200 mg | | |
|---|---|---|---|
| | CLL | MCL | WM |
| Response Category | (N = 20) | (N = 18)^{a} | (N = 19) |
| Median follow-up, mo. (range) | 7 (1-12) | 7 (1-12) | 7 (1-12) |
| Complete response, N (%) | — | — | — |
| Very good partial response, N (%) | — | — | — |
| Partial response, N (%) | 19 (95)^{b} | 9 (50) | 12 (63) |
| Minor response, N (%) | — | — | 6 (32) |
| Stable disease, N (%) | 1 (5) | 6 (33) | — |
| Progressive disease, N (%) | — | 3 (17) | 1 (5) |
| Not evaluable, N (%) | — | — | — |
| ORR | 95% | 50% | 95% |

^{a}One patient does not have measurable disease and is excluded from the denominator.
^{b}Includes PR and PR except persistent lymphocytosis (PR-L).

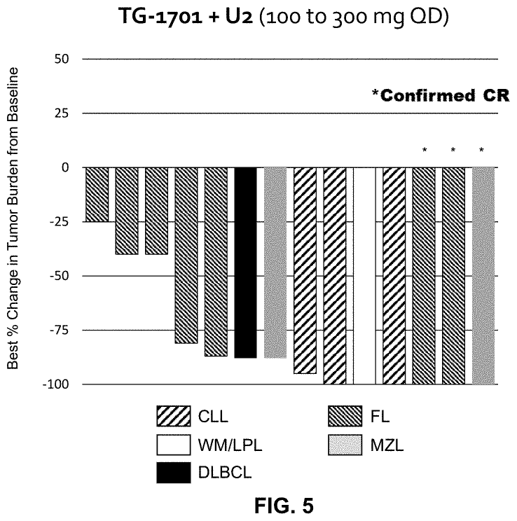

Efficacy results with respect to TG-1701+U2 dose-escalation are depicted in Table 7 and FIG. 5.

TABLE 7
Efficacy-TG-1701 + U2 Dose-Escalation

| | TG-1701 + U2 |
|---|---|
| Response Category | (N = 14) |
| Median follow-up, mo (range) | 12 (1-18) |
| Complete response, N (%) | 3 (21.5) |
| Very good partial response, N (%) | 1 (7) |
| Partial response, N (%) | 7 (50) |
| Minor response, N (%) | — |
| Stable disease, N (%) | 3 (21.5) |
| Progressive disease, N (%) | — |
| Not evaluable, N (%) | — |
| ORR | 79% |

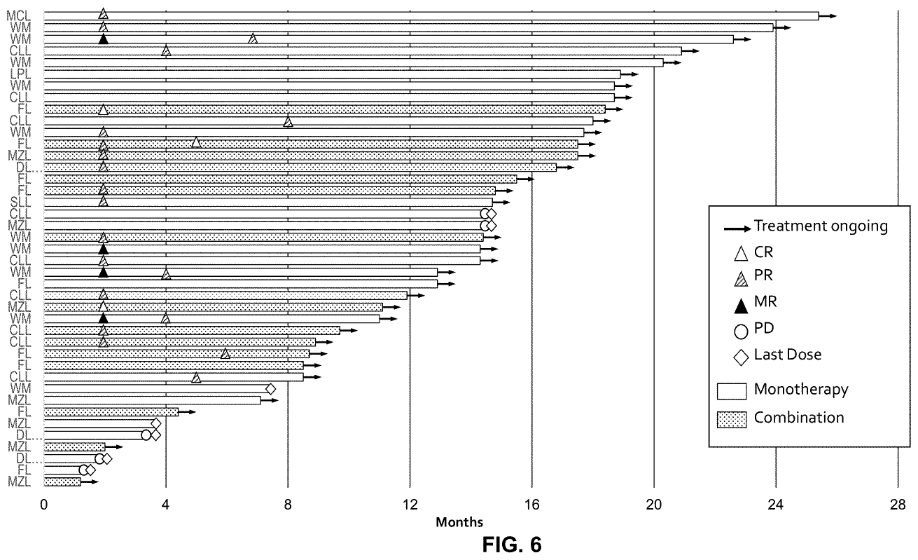

FIG. 6 shows treatment exposure and response duration in 41 patients in the dose-escalation phase of the TG-1701 monotherapy and the TG-1701+U2 combination arms. The patients included: DLBCL=3 patients; WM=10 patients; FL=9 patients; SLL=1 patient; LPL=1 patient; MZL=7 patients; MCL=1 patient; CLL=9 patients. FIG. 6 shows that 3 patients had a complete response (CR) following combination therapy with TG-1701+U2. FIG. 6 also shows that 20 patients had a partial response (PR).

119 patients have been treated with TG-1701 as follows:

25 in the monotherapy DE arm; 61 in the 200 mg QD expansion cohort (20 CLL [5 TN], 21 MCL [3 TN], and 20 WM [8 TN]); 17 in the 300 mg QD expansion cohort (all R/R CLL); and 16 in the 1701+U2 DE arm (all R/R).

Patients in the DE arms and expansion cohorts had a median of 1 prior systemic therapy (range: 1-10).

TG-1701 was well-tolerated and the maximum tolerated dose was not reached up through 400 mg QD, with DE proceeded until 3 dose levels above complete BTK occupancy (≥99% occupancy observed at 100 mg QD). One dose-limiting toxicity was observed, a grade (G)3 ALT increased at 400 mg QD monotherapy. This event was transient, the patient remained asymptomatic with normal liver function, and continued treatment at a reduced dose of 300 mg QD. The most common G≥3 adverse events were neutropenia (8%) on monotherapy, and neutropenia and ALT/AST increased for 1701+U2 (12% and 25%, respectively). There were no G4 AEs with TG-1701 monotherapy. Among all-causality AEs of special interest, atrial fibrillation occurred in 5 patients (4%), G≥3 hypertension occurred in 1 patient (1%), and bleeding events (e.g., bruising, petechiae) occurred in 23 patients (19%, all G1-G2). No cases of ventricular tachyarrhythmia have been reported. There have been no treatment-related deaths nor treatment discontinuations due to AEs. Linear kinetics are apparent, evidenced by approximately dose proportional increase in exposure.

With a median follow up of 7 months in the 200 mg QD monotherapy expansion cohorts, preliminary overall response rates (ORR) were as follows: 95% (19/20) in CLL; 50% (9/18) in MCL; and 95% (18/19) in WM. No complete responses (CR) were confirmed in patients on TG-1701 monotherapy.

The ORR for TG-1701+U2 combination therapy was 79% (11/14), including 1/1 WM; 3/3 CLL; 4/7 FL; 2/2 MZL; and 1/1 DLBCL) with a 21% CR rate. Efficacy is reflected in the best percent change from baseline in disease burden in all patients who had received at least one post baseline scan to assess disease/tumor burden, with responses determined according to standard international working group criteria for NHL and CLL, as set forth in Cheson, B. D. et al., J Clin Oncol 25:579-586 (2007) and Hallek, M. et al., Blood 111:5446-5456 (2008), respectively. The best percent change in tumor burden from baseline for patients in the 1701+U2 combination arm is shown in the bar graph of FIG. 5. Treatment exposure and response duration for patients in the 1701+U2 combination arm (and the TG-1701 monotherapy arm) are presented in FIG. 6.

### Conclusions

Results are reported of a unique Phase 1/1b parallel dose-escalation study of TG-1701 monotherapy and TG-1701 in combination with umbralisib and ublituximab (TG-1701+U2) in patients with various B-cell malignancies. TG-1701 was safe and effective in patients with B-cell malignancies and demonstrated near 100% saturation of the BTK at all dose levels studied. Monotherapy with TG-1701 showed a low incidence of off-target adverse events. The combination of 1701+U2 in patients with B-cell malignancy has shown promising clinical activity, including early complete responses (CRs), and enhanced depth of response over TG-1701 monotherapy with no additional toxicity. This study (NCT03671590) continues enrollment.

### Example 2: Clinical Activity of TG-1701, as Monotherapy and in Combination with Ublituximab and Umbralisib (“U2”), in Patients with Chronic Lymphocytic Leukemia (CLL)

### Background

TG-1701 is a selective, covalent BTK inhibitor administered once daily (QD). Both the “U2” combination (anti-CD20 mAb ublituximab+the PI3Kδ-CK1ε inhibitor umbralisib) and BTK inhibitors are highly efficacious in treatment-naïve (TN) and relapsed/refractory (R/R) CLL, each having previously demonstrated superiority over standard chemoimmunotherapy. In this Example, results are reported for patients treated with TG-1701 alone or in combination with U2 from the ongoing Phase 1 study discussed in Examples 1 and 3, with a focus on patients with CLL.

### Methods

As discussed in Examples 1 and 3, patients with R/R CLL and B-cell non-Hodgkin lymphoma (NHL) were enrolled in an ongoing Phase 1 study initially evaluating dose escalation (DE) of oral TG-1701 each day (QD) continuously administered in 28-day cycles (100, 200, 300, and 400 mg). After characterizing the safety profile of TG-1701 monotherapy, a parallel DE arm of TG-1701+U2 was implemented. Select dose levels of TG-1701 monotherapy were also expanded. All patients were treated until disease progression, unacceptable toxicity, or investigator/patient decision to withdraw. Safety was evaluated in all treated patients, and efficacy was evaluated in all treated patients with CLL who had at least 1 post-baseline assessment.

### Results

At the time of data cutoff, 127 patients were treated with TG-1701, 50 of whom had CLL, which is the focus of this Example. Enrollment was as follows: 25 patients in the monotherapy DE arm (6 with CLL); 61 in the 200-mg disease-specific cohorts (20 with CLL [5 TN], 21 with mantle cell lymphoma (MCL), [4 TN], 20 with Waldenstrom's macroglobulinemia (WM), 8 TN]); 20 in the 300-mg CLL cohort [4 TN]; and 21 in the 1701+U2 DE arm (4 with CLL). Patients with MCL or WM in the 200-mg disease-specific cohorts were excluded from this analysis. The median number of prior therapies among CLL patients was 1 (range, 0-7), and all patients were BTKi-naïve. TG-1701 was well tolerated and the maximum tolerated dose for monotherapy was not reached up to 400 mg (near 100% saturation of the BTK at all dose levels studied).

Table 8 describes the patient demographics and disease characteristics for the 50 CLL patients studied.

Table 9 describes the patient disposition for the 50 CLL patients studied.

TABLE 8
Patient Demographics and Disease Characteristics-CLL Patients

| | Dose-Escalation Phase | | Dose-Expansion | |
|---|---|---|---|---|
| | TG-1701 | TG-1701 + | Cohorts | |
| | 100- | U2 | TG-1701 | TG-1701 |
| | 400 mg | 100-300 mg | 200 mg | 300 mg |
| Characteristic | N = 6 | N = 4 | N = 20 | N = 20 |
| Male sex, N(%) | 4 (67) | 3 (75) | 7 (35) | 10 (50) |
| Age, years, median | 63 (57/83) | 60 (47/70) | 70 (53-86) | 71 (49-80) |
| (min/max) ≥ 75 | 2 (33) | — | 3 (15) | 6 (30) |
| years, N(%) | | | | |
| ECOG 0/1/2 (%) | 50/50/0 | 75/25/0 | 35/65/0 | 30/70/0 |
| Prior therapies, | 1 (1-2) | 1 (1-2) | 1 (0-4) | 1 (0-7) |
| median (range)* | | | | |
| Refractory to last | — | 1 (25) | 3 (15) | 2 (10) |
| prior therapy, N(%) | | | | |
| Treatment-naïve, | — | — | 5 (25) | 4 (20) |
| N(%) | | | | |
| High Risk features | | | | |
| Unmutated IGHV | 80% (4/5) | 100% (4/4) | 41% (7/17) | 72% (13/18) |
| status % (n/N) | | | | |
| Del 17p or TP53 | 50% (3/6) | 50% (2/4) | 5% (1/19) | 20% (4/20) |
| mutation % (n/N) | | | | |
| Del 17P & TP 53 | 50% (2/4) | 50% (2/4) | 6% (1/17) | 23% (3/13) |
| mutation % (n/N) | | | | |

*Calculation excludes treatment-naïve patients.

TABLE 9
Patient Disposition-CLL

| | Dose-Escalation Phase | | Dose-Expansion Cohorts | |
|---|---|---|---|---|
| | TG-1701 | TG-1701 + U2 | TG-1701 | TG-1701 |
| | 100-400 mg | 100-300 mg | 200 mg | 300 mg |
| | N = 6 | N = 4 | N = 20 | N = 20 |
| Pts continuing treatment, | 4 (67) | 4 (100) | 18 (90) | 18 (90) |
| N(%) | | | | |
| Dose reduction (any agent), | 3 (50) | — | 1 (5) | — |
| N(%) | | | | |
| Pts discontinued treatment, | 2 (33) | — | 2 (10) | 2 (10) |
| N(%) | | | | |
| Reason for treatment | | | | |
| discontinuation, N(%) | | | | |
| Progression by iwCLL | 1 | — | — | — |
| criteria | | | | |
| Clinical progression | — | — | — | — |
| Due to AE | — | — | — | — |
| Pt/physician decision | — | — | 1 | — |
| Death | — | — | 1^{‡} | 2^{‡} |
| Other | 1* | — | — | — |

*Intercurrent illness developed which compromises further participation in the study.
^{‡}Death due to SARS-CoV-2 infection.

Table 10 presents the safety results for the 50 CLL patients studied.

TABLE 10
Safety—All-Causality AEs ≥15% in Either of the Dose-Expansion Cohorts

| | Dose-Escalation Phase | | | | Dose-Expansion Cohorts | | | |
|---|---|---|---|---|---|---|---|---|
| | TG-1701 | | TG-1701 + U2 | | TG-1701 | | TG-1701 | |
| | 100-400 mg | | 100-300 mg | | 200 mg | | 300 mg | |
| | N = 6 | | N = 4 | | N = 20 | | N = 20 | |
| Adverse event, N | Any | Grade | Any | Grade | Any | Grade | Any | Grade |
| (%)^{§} | Grade | ≥3 | Grade | ≥3 | Grade | ≥3 | Grade | ≥3 |
| Contusion | 4 | — | — | — | 4 (20) | — | 1 (5) | — |
| Diarrhea | — | — | 1 | — | 4 (20) | — | 2 (10) | — |
| URTI | 3 | — | — | — | 2 (10) | — | 3 (15) | — |
| Nausea | 1 | — | 1 | — | — | — | 3 (15) | — |
| COVID-19 | — | — | — | — | 1 (5) | — | 3 (15) | 1 (5) |

Hematologic & Lab Abnormalities

| | | | | | | | | |
|---|---|---|---|---|---|---|---|---|
| Neutropenia | 1 | 1 | — | — | 2 (10) | 2 (10) | 4 (20) | 4 (20) |
| ALT increased | 2 | 1 | — | — | 4 (20) | — | 3 (15) | 1 (5) |
| AST increased | 1 | 1 | — | — | 2 (10) | — | 3 (15) | 1 (5) |
| Anemia | — | — | 1 | — | 3 (15) | 1 (5) | — | — |

BTKi AEs of Special Interest

| | | | | | | | | |
|---|---|---|---|---|---|---|---|---|
| Arthralgia | 1 | — | — | — | 1 (5) | 1 (5) | 1 (5) | — |
| Atrial fibrillation | 1 | 1 | — | — | — | — | — | — |
| Hypertension | — | — | — | — | 1 (5) | — | 1 (5) | — |

^{§}Dose-escalation data reported as n patients with respective AEs

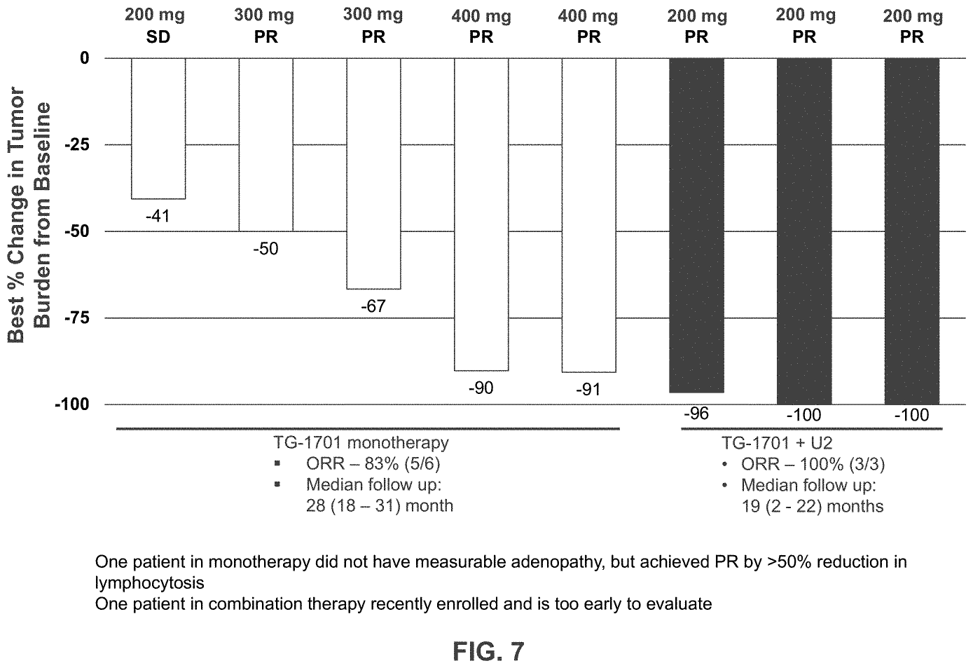

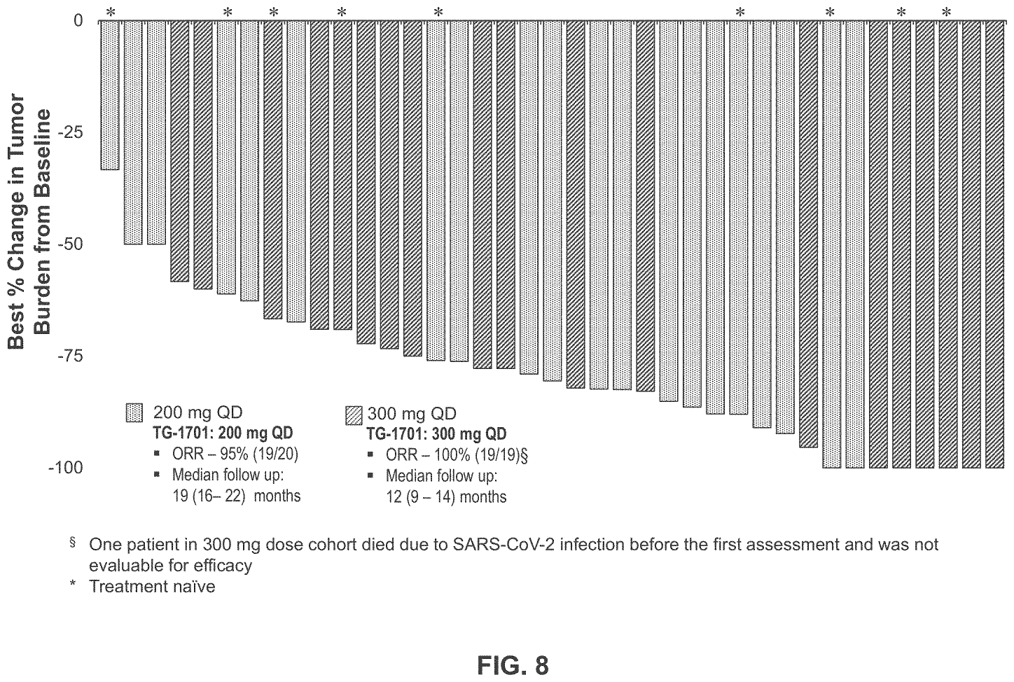

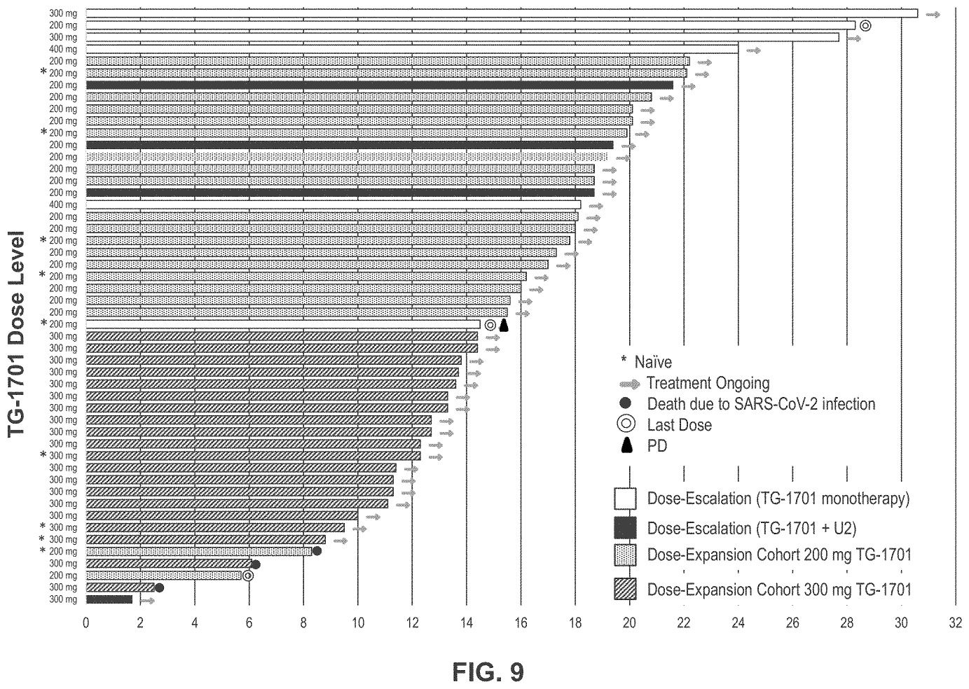

Efficacy: At the data cut-off, 48 patients with CLL were evaluable for response, including 9 in DE. The ORR was 95.6% for TG-1701 monotherapy (all PR/PR-L) and 100% for TG-1701+U2 (all PR). The median duration of response has not been reached in either cohort. For the 9 efficacy evaluable CLL patients treated in the DE cohorts, the best change from baseline in tumor burden and respectice responses are presented in FIG. 7. For the 39 efficacy evaluable CLL patients treated in the dose-expansion cohorts, the best change from baseline in tumor burden and respective responses are presented in FIG. 8. The treatment duration and disposition for all 50 CLL patients are presented in FIG. 9. In patients with anemia and thrombocytopenia at baseline, sustained improvement in hematologic variables was observed in the 200-mg and 300-mg cohorts. Lymphocytosis was observed in 70% of the monotherapy patients, with resolution to normal or less than 50% of baseline in 57.1%. Consistent response rates were observed across all subgroups, including age and high-risk genomic features such as del17p/TP53, unmutated immunoglobulin heavy-chain variable-region (IGHV), and complex karyotype (defined as 3 or more cytogenetic abnormalities).

### Conclusion

TG-1701 exhibited an encouraging safety and efficacy profile in patients with CLL, with promising activity and a manageable tolerability profile as monotherapy and in combination with U2 (FIG. 7). Future registration trials are being planned in CLL with TG-1701. Recruitment to this study (NCT03671590) continues.

### Example 3: Updated Clinical Activity of TG-1701 as Monotherapy and in Combination with Ublituximab and Umbralisib (“U2”) in Patients with B-Cell Malignancies

### Background

TG-1701 is an irreversible, selective, novel Bruton's tyrosine kinase inhibitor (BTKi) administered once daily (QD). BTKis, as well as the U2 combination (anti-CD20 mAb ublituximab+the PI3Kδ-CK1ε inhibitor umbralisib), are highly efficacious in CLL, each of which have been previously demonstrated to be superior over standard chemoimmunotherapy. Treatment with a more selective BTK inhibitor could result in improved efficacy and safety outcomes compared with ibrutinib (Hillmen, P. et al., “First interim analysis of ALPINE study: Results of a phase 3 randomized study of zanubrutinib vs ibrutinib in patients with relapsed/refractory chronic lymphocytic leukemia/small lymphocytic lymphoma,” presented at: 2021 European Hematology Association Congress; Jun. 9-17, 2021; Virtual. Abstract LB1900.). Dual blockade of the B-cell receptor (BCR) pathway through the combination of TG-1701 with U2 may confer a greater depth of response compared to either regimen alone.

In this Example, updated results are reported for patients with R/R CLL and B-cell NHL treated with TG-1701 in combination with U2, from the ongoing Phase 1 clinical trial discussed above in Example 1.

### Methods

Patients with R/R CLL and non-Hodgkin lymphoma (NHL) were enrolled in an ongoing Phase 1 study, as described in Example 1. After characterizing the safety profile of TG-1701 monotherapy, a parallel dose escalation arm of TG-1701+U2 was implemented. Select dose levels of TG-1701 monotherapy and TG-1701+U2 were also expanded. All patients were treated until disease progression, unacceptable toxicity, or investigator/patient decision to withdraw. Safety was evaluated in all treated patients, and efficacy was evaluated in all treated patients who had at least 1 post-baseline assessment. In this Example, data from the TG-1701+U2 dose escalation/expansion cohort is presented.

### Results

Over a study period of 33.4 months, 142 patients were treated with TG-1701, 36 of whom were enrolled in the TG-1701+U2 arm. The median number of prior therapies across all treated patients was 1 (range, 0-10) and all patients were BTKi-naïve.

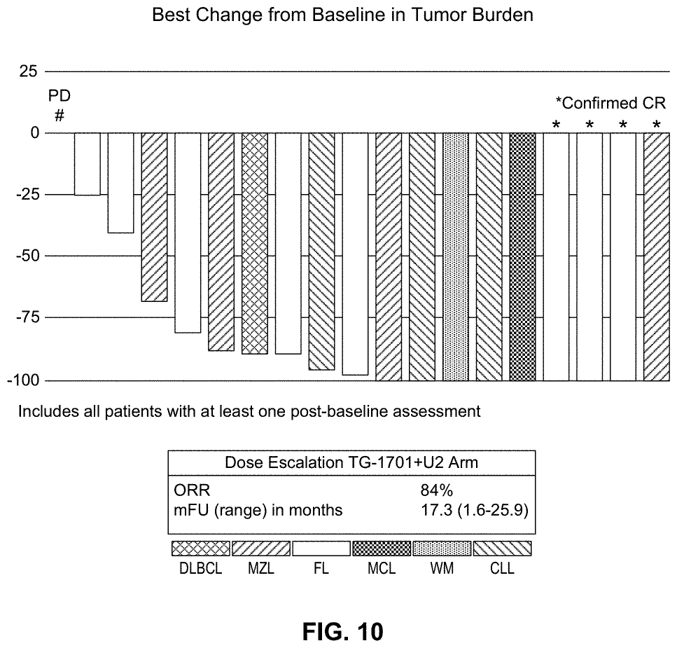

Among the 36 patients treated with TG-1701+U2, 19 were evaluable for efficacy and safety (17 were too early to evaluate). The median age was 69 years (range 47-81), and 56% were male. TG-1701+U2 was well tolerated at 4 different dose levels without dose-limiting toxicities. The most common (>30%) all-causality, all grade treatment-emergent adverse events (TEAEs) were diarrhea (53%), contusion (42%), nausea (37%), hypertension, ALT/AST increase, and fatigue (all 32% each) with TG-1701+U2. Grade 3/4 AEs>15% were limited to ALT/AST increase (21%). Dose reduction occurred in 1 patient due to an AE, and 4 patients discontinued at least 1 study drug due to an AE: 2 discontinued umbralisib, 1 discontinued umbralisib and TG-1701, and 1 discontinued all 3 agents. At the data cut-off, the overall response rate (ORR) was 84% (4 CR and 12 PR) among 19 evaluable patients, with remaining patients awaiting post-baseline assessment. See, FIG. 10. The median duration of response has not been reached.

### Conclusions

TG-1701 exhibits an encouraging safety and efficacy profile as monotherapy in patients with CLL and additionally shows promising activity and a manageable tolerability profile in combination with U2. Recruitment to this study (NCT03671590) continues.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific aspects will so fully reveal the general nature of the invention that others can, by applying knowledge within the skill of the art, readily modify and/or adapt for various applications such specific aspects, without undue experimentation, without departing from the general concept of the present invention. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The contents of all cited references (including literature references, patents, patent applications, and websites) that can be cited throughout this application are hereby expressly incorporated by reference in their entirety for any purpose, as are the references cited therein.

1-54. (canceled)

55. A method of treating a B-cell malignancy in a subject in need thereof, comprising,
(a) administering to the subject a combination of agents, in therapeutically effective amounts, said combination of agents comprising:
(i) a PI3K-delta selective inhibitor of Formula A, or a stereoisomer thereof, or a pharmaceutically acceptable salt, solvate, or prodrug thereof: 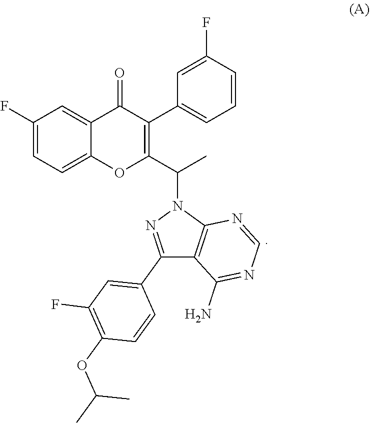 [SMILES: CC(C)Oc1ccc(-c2nn(C(C)c3oc4ccc(F)cc4c(=O)c3-c3cccc(F)c3)c3ncnc(N)c23)cc1F]
selected from one or more of:
(RS)-2-(1-(4-amino-3-(3-fluoro-4-isopropoxyphenyl)-1H-pyrazolo[3,4-d]pyrimidin-1-yl)ethyl)-6-fluoro-3-(3-fluorophenyl)-4H-chromen-4-one;
(S)-2-(1-(4-amino-3-(3-fluoro-4-isopropoxyphenyl)-1H-pyrazolo[3,4-d]pyrimidin-1-yl)ethyl)-6-fluoro-3-(3-fluorophenyl)-4H-chromen-4-one; and
(R)-2-(1-(4-amino-3-(3-fluoro-4-isopropoxyphenyl)-1H-pyrazolo[3,4-d]pyrimidin-1-yl)ethyl)-6-fluoro-3-(3-fluorophenyl)-4H-chromen-4-one;
(ii) an anti-CD20 antibody, wherein the anti-CD20 antibody is ublituximab or an anti-CD20 antibody or antibody fragment that binds to the same epitope as ublituximab; and
(iii) a BTK inhibitor, wherein the BTK inhibitor is (R)-4-amino-1-(1-(but-2-ynoyl)pyrrolidin-3-yl)-3-(4-(2,6-difluorophenoxy)phenyl)-1,6-dihydro-7H-pyrrolo[2,3-d]pyridazin-7-one (TG-1701), or an isomer, polymorph, enantiomer, pharmaceutically acceptable salt, solvate, or prodrug thereof; and
(b) treating said subject with a B-cell malignancy.

56. The method of claim 55, wherein the PI3K-delta selective inhibitor is administered orally at a dosage from: about 200 mg to about 1200 mg, about 400 mg to about 1000 mg, about 400 mg to about 800 mg, about 400 mg, about 500 mg, about 600 mg, about 700 mg, about 800 mg, about 900 mg, about 1000 mg, or about 1200 mg per day.

57. The method of claim 55, wherein the PI3K-delta selective inhibitor is (S)-2-(1-(4-amino-3-(3-fluoro-4-isopropoxyphenyl)-1H-pyrazolo[3,4-d]pyrimidin-1-yl)ethyl)-6-fluoro-3-(3-fluorophenyl)-4H-chromen-4-one (umbralisib).

58. The method of claim 55, wherein the PI3K-delta selective inhibitor is (S)-2-(1-(4-amino-3-(3-fluoro-4-isopropoxyphenyl)-1H-pyrazolo[3,4-d]pyrimidin-1-yl)ethyl)-6-fluoro-3-(3-fluorophenyl)-4H-chromen-4-one p-toluenesulfonic acid salt (umbralisib PTSA salt).

59. The method of claim 55, wherein the umbralisib is administered orally at a dose of about 400 mg per day, about 600 mg per day, or about 800 mg per day.

60. The method of claim 59, wherein the umbralisib is administered orally at a dose of 800 mg per day.

61. The method of claim 55, wherein the anti-CD20 antibody is ublituximab and comprises the VH CDR1, CDR2, and CDR3 region of sequences SEQ ID NOS: 1, 2, and 3, and the VL CDR1, CDR2, and CDR3 region of sequences SEQ ID NOS: 6, 7, and 8.

62. The method of claim 61, wherein ublituximab comprises the VH of SEQ ID NO: 4 and the VL of SEQ ID NO: 9.

63. The method of claim 61, wherein ublituximab is administered intravenously at a dose from: about 450 mg to about 1200 mg, about 600 to about 1200 mg, about 600 to about 1000 mg, about 600 to about 900 mg, about 600 mg, about 700 mg, about 800 mg, or about 900 mg about once every 1 to 9 weeks, about once every week, about twice every week, about once every 2 weeks, about once every 3 weeks, about once every 4 weeks, about once every 5 weeks, about once every 6 weeks, about once every 7 weeks, about once every 8 week, or about once every 9 weeks.

64. The method of claim 63, wherein the ublituximab is administered at a dose of about 900 mg.

65. The method of claim 63, wherein said ublituximab is administered on days 1, 8, and 15 of cycle 1 and day 1 of cycles 2, 3, 4, 5, 6, and every 3 months thereafter, wherein each cycle is about 28 days.

66. The method claim 55, wherein the TG-1701 or an isomer, polymorph, enantiomer, pharmaceutically acceptable salt, solvate, or prodrug thereof is administered orally once daily at a dosage from: about 100 mg, about 200 mg, about 300 mg, or about 400 mg.

67. The method of claim 66, wherein the TG-1701 or an isomer, polymorph, enantiomer, pharmaceutically acceptable salt, solvate, or prodrug thereof is administered orally once daily at a dosage of about 300 mg per day or about 400 mg per day.

68. The method of claim 55, wherein said subject is a human subject.

69. The method of claim 68, wherein said human subject has a B-cell malignancy selected from the group consisting of acute lymphocytic leukemia (ALL), acute myeloid leukemia (AML), chronic lymphocytic leukemia (CLL), small lymphocytic lymphoma (SLL), multiple myeloma (MM), non-Hodgkin's lymphoma (NHL), mantle cell lymphoma (MCL), follicular lymphoma (FL), Waldenstrom's macroglobulinemia (WM), diffuse large B-cell lymphoma (DLBCL), marginal zone lymphoma (MZL), Burkitt's lymphoma, hairy cell leukemia (HCL), and Richter's transformation (RT).

70. The method of claim 69, wherein the B-cell malignancy is selected from the group consisting of chronic lymphocytic leukemia (CLL), follicular lymphoma (FL), mantle cell lymphoma (MCL), small lymphocytic lymphoma (SLL), diffuse large B-cell lymphoma (DLBCL), Waldenstrom's macroglobulinemia (WM), and marginal zone lymphoma (MZL).

71. The method of claim 70, wherein the B-cell malignancy is CLL.

72. The method of claim 69, wherein the B-cell malignancy is refractory to an anti-CD20 antibody, a PI3K-delta inhibitor, or a BTK inhibitor, administered as a monotherapy.

73. The method of claim 72, wherein the B-cell malignancy is refractory to a non-umbralisib PI3K-delta inhibitor, a non-ublituximab anti-CD20 antibody, or a non-TG-1701 BTK inhibitor.

74. The method of claim 55, wherein the B-cell malignancy has relapsed.

75. The method of claim 55, wherein a complete or partial anti-tumor response is observed following administration of all agents i, ii, and iii to said subject.

76. The method of claim 75, wherein the duration of the anti-tumor response is about 24 weeks to about 36 months.

77. The method of claim 75, wherein the anti-tumor response is determined by percent reduction in tumor burden from baseline, and wherein the percent reduction in tumor burden from baseline is about 25-100%.

78. The method of claim 77, wherein the percent reduction in tumor burden from baseline is at least about 50%.

79. The method of claim 55, further comprising administering at least one additional therapeutic agent, wherein the at least one additional therapeutic agent is selected from the group consisting of mitotic inhibitors, alkylating agents, anti-metabolites, anthracyclines, vinca alkaloids, plant alkaloids, nitrogen mustards, proteasome inhibitors, intercalating antibiotics, growth factor inhibitors, cell-cycle inhibitors, biological response modifiers, anti-hormones, angiogenesis inhibitors, anti-androgens, DNA interactive agents, purine analogues, topoisomerase I inhibitors, topoisomerase II inhibitors, tubulin interacting agents, hormonal agents, thymidilate synthase inhibitors, non-BTK and non-PI3K-delta tyrosine kinase inhibitors, angiogenesis inhibitors, EGF inhibitors, VEGF inhibitors, CDK inhibitors, SRC inhibitors, c-Kit inhibitors, Her1/2 inhibitors, BET bromodomain inhibitors, inhibitors of myc, anti-tumor antibodies, monoclonal antibodies directed against growth factor receptors, monoclonal antibodies directed against checkpoint inhibitors, monoclonal antibodies against CD19 and/or CD47, protein kinase modulators, radioactive isotopes, immunotherapies, glucocorticoids, and any combinations thereof.

80. A kit comprising: (a) a combination of agents (i)-(iii) of claim 55; (b) instructions for using said agents in combination, and (c) optionally, one or more additional therapeutic agents that can be used to treat B-cell malignancies.
